SEQUENCE LISTING (113 sequences)

SEQ ID NO: 1 - protein, 446 aa, Artificial sequence
  Sequence is synthesized
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 116 aa, Artificial sequence
  Sequence is synthesized
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 3 - protein, 5 aa, Artificial sequence
  Sequence is synthesized
DYGMH

SEQ ID NO: 4 - protein, 17 aa, Artificial sequence
  Sequence is synthesized
YISSGSSTIYYADTVKG

SEQ ID NO: 5 - protein, 7 aa, Artificial sequence
  Sequence is synthesized
RGLLLDY

SEQ ID NO: 6 - protein, 19 aa, Artificial sequence
  Sequence is synthesized
MGWSCIILFLVATATGVHS

SEQ ID NO: 7 - protein, 218 aa, Artificial sequence
  Sequence is synthesized
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 111 aa, Artificial sequence
  Sequence is synthesized
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIK

SEQ ID NO: 9 - protein, 15 aa, Artificial sequence
  Sequence is synthesized
RASKSVSTSSYSYMH

SEQ ID NO: 10 - protein, 7 aa, Artificial sequence
  Sequence is synthesized
YASYLES

SEQ ID NO: 11 - protein, 9 aa, Artificial sequence
  Sequence is synthesized
QHSREFPWT

SEQ ID NO: 12 - protein, 19 aa, Artificial sequence
  Sequence is synthesized
MGWSCIILFLVATATGVHS

SEQ ID NO: 13 - protein, 446 aa, Artificial sequence
  Sequence is synthesized
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 116 aa, Artificial sequence
  Sequence is synthesized
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 15 - protein, 5 aa, Artificial sequence
  Sequence is synthesized
NYGMH

SEQ ID NO: 16 - protein, 5 aa, Artificial sequence
  Sequence is synthesized
  misc_feature (1)..(1): Xaa at position 1 is D or N
XYGMH

SEQ ID NO: 17 - protein, 17 aa, Artificial sequence
  Sequence is synthesized
YISSGSSTIYYADTVKG

SEQ ID NO: 18 - protein, 7 aa, Artificial sequence
  Sequence is synthesized
RGLLLDY

SEQ ID NO: 19 - protein, 19 aa, Artificial sequence
  Sequence is synthesized
MGWSCIILFLVATATGVHS

SEQ ID NO: 20 - protein, 218 aa, Artificial sequence; identical to SEQ ID NO: 7
  Sequence is synthesized

SEQ ID NO: 21 - protein, 111 aa, Artificial sequence; identical to SEQ ID NO: 8
  Sequence is synthesized

SEQ ID NO: 22 - protein, 15 aa, Artificial sequence
  Sequence is synthesized
RASKSVSTSSYSYMH

SEQ ID NO: 23 - protein, 7 aa, Artificial sequence
  Sequence is synthesized
YASYLES

SEQ ID NO: 24 - protein, 9 aa, Artificial sequence
  Sequence is synthesized
QHSREFPWT

SEQ ID NO: 25 - protein, 19 aa, Artificial sequence
  Sequence is synthesized
MGWSCIILFLVATATGVHS

SEQ ID NO: 26 - protein, 17 aa, Artificial sequence
  Sequence is synthesized
YISSGSSTIYYADSVKG

SEQ ID NO: 27 - protein, 17 aa, Artificial sequence
  Sequence is synthesized
YISSSSSTIYYADSVKG

SEQ ID NO: 28 - protein, 15 aa, Artificial sequence
  Sequence is synthesized
RASKSVSTSSYSYLA

SEQ ID NO: 29 - protein, 15 aa, Artificial sequence
  Sequence is synthesized
RASKTVSTSSYSYMH

SEQ ID NO: 30 - protein, 7 aa, Artificial sequence
  Sequence is synthesized
YASYLQS

SEQ ID NO: 31 - protein, 17 aa, Artificial sequence
  Sequence is synthesized
  misc_feature (5)..(5): Xaa at position 5 may be any naturally-occurring amino acid
  misc_feature (14)..(14): Xaa at position 14 may be any naturally-occurring amino acid
YISSXSSTIYYADXVKG

SEQ ID NO: 32 - protein, 15 aa, Artificial sequence
  Sequence is synthesized
  misc_feature (5)..(5): Xaa at position 5 may be any naturally-occurring amino acid
  misc_feature (14)..(14): Xaa at position 14 may be any naturally-occurring amino acid
  misc_feature (15)..(15): Xaa at position 15 may be any naturally-occurring amino acid
RASKXVSTSSYSYXX

SEQ ID NO: 33 - protein, 7 aa, Artificial sequence
  Sequence is synthesized
  misc_feature (6)..(6): Xaa at position 6 may be any naturally-occurring amino acid
YASYLXS

SEQ ID NO: 34 - protein, 5 aa, Artificial sequence
  Sequence is synthesized
QYGMH

SEQ ID NO: 35 - protein, 5 aa, Artificial sequence
  Sequence is synthesized
EYGMH

SEQ ID NO: 36 - protein, 5 aa, Artificial sequence
  Sequence is synthesized
  misc_feature (1)..(1): Xaa at position 1 is D or N
  misc_feature (5)..(5): Xaa at position 5 is K or R
XYGMX

SEQ ID NO: 37 - protein, 5 aa, Artificial sequence
  Sequence is synthesized
  misc_feature (5)..(5): Xaa at position 5 is K or R
QYGMX

SEQ ID NO: 38 - protein, 5 aa, Artificial sequence
  Sequence is synthesized
  misc_feature (5)..(5): Xaa at position 5 is K or R
EYGMX

SEQ ID NO: 39 - protein, 17 aa, Artificial sequence
  Sequence is synthesized
YISSGSSTIYYAETVKG

SEQ ID NO: 40 - protein, 17 aa, Artificial sequence
  Sequence is synthesized
  misc_feature (16)..(16): Xaa at position 16 is R or H
YISSGSSTIYYADTVXG

SEQ ID NO: 41 - protein, 17 aa, Artificial sequence
  Sequence is synthesized
  misc_feature (16)..(16): Xaa at position 16 is R or H
YISSGSSTIYYAETVXG

SEQ ID NO: 42 - protein, 7 aa, Artificial sequence
  Sequence is synthesized
  misc_feature (1)..(1): Xaa at position 1 is K or H
XGLLLDY

SEQ ID NO: 43 - protein, 7 aa, Artificial sequence
  Sequence is synthesized
RGLLLEY

SEQ ID NO: 44 - protein, 7 aa, Artificial sequence
  Sequence is synthesized
  misc_feature (1)..(1): Xaa at position 1 is K or H
XGLLLEY

SEQ ID NO: 45 - protein, 15 aa, Artificial sequence
  Sequence is synthesized
  misc_feature (1)..(1): Xaa at position 1 is K or H
XASKSVSTSSYSYMH

SEQ ID NO: 46 - protein, 15 aa, Artificial sequence
  Sequence is synthesized
  misc_feature (4)..(4): Xaa at position 4 is R or H
RASXSVSTSSYSYMH

SEQ ID NO: 47 - protein, 15 aa, Artificial sequence
  Sequence is synthesized
  misc_feature (15)..(15): Xaa at position 15 is K or R
RASKSVSTSSYSYMX

SEQ ID NO: 48 - protein, 15 aa, Artificial sequence
  Sequence is synthesized
  misc_feature (1)..(1): Xaa at position 1 is K or H
  misc_feature (4)..(4): Xaa at position 4 is R or H
XASXSVSTSSYSYMH

SEQ ID NO: 49 - protein, 15 aa, Artificial sequence
  Sequence is synthesized
  misc_feature (1)..(1): Xaa at position 1 is K or H
  misc_feature (15)..(15): Xaa at position 15 is K or R
XASKSVSTSSYSYMX

SEQ ID NO: 50 - protein, 15 aa, Artificial sequence
  Sequence is synthesized
  misc_feature (4)..(4): Xaa at position 4 is R or H
  misc_feature (15)..(15): Xaa at position 15 is K or R
RASXSVSTSSYSYMX

SEQ ID NO: 51 - protein, 7 aa, Artificial sequence
  Sequence is synthesized
YASYLDS

SEQ ID NO: 52 - protein, 9 aa, Artificial sequence
  Sequence is synthesized
  misc_feature (2)..(2): Xaa at position 2 is K or R
QXSREFPWT

SEQ ID NO: 53 - protein, 9 aa, Artificial sequence
  Sequence is synthesized
  misc_feature (4)..(4): Xaa at position 4 is K or H
QHSXEFPWT

SEQ ID NO: 54 - protein, 9 aa, Artificial sequence
  Sequence is synthesized
QHSRDFPWT

SEQ ID NO: 55 - protein, 9 aa, Artificial sequence
  Sequence is synthesized
  misc_feature (2)..(2): Xaa at position 2 is K or R
  misc_feature (4)..(4): Xaa at position 4 is K or H
QXSXEFPWT

SEQ ID NO: 56 - protein, 9 aa, Artificial sequence
  Sequence is synthesized
  misc_feature (2)..(2): Xaa at position 2 is K or R
QXSRDFPWT

SEQ ID NO: 57 - protein, 9 aa, Artificial sequence
  Sequence is synthesized
  misc_feature (4)..(4): Xaa at position 4 is K or H
QHSXDFPWT

SEQ ID NO: 58 - protein, 5 aa, Artificial sequence
  Sequence is synthesized
  misc_feature (1)..(1): Xaa at position 1 is any of D, E, N, Q, R, and K
  misc_feature (5)..(5): Xaa at position 5 is any one of K, R, and H
XYGMX

SEQ ID NO: 59 - protein, 17 aa, Artificial sequence
  Sequence is synthesized
  misc_feature (5)..(5): Xaa at position 5 is G or S
  misc_feature (13)..(13): Xaa at position 13 is D or E
  misc_feature (14)..(14): Xaa at position 14 is T or S
  misc_feature (16)..(16): Xaa at position 16 is any one of K, R, and H
YISSXSSTIYYAXXVXG

SEQ ID NO: 60 - protein, 7 aa, Artificial sequence
  Sequence is synthesized
  misc_feature (1)..(1): Xaa at position 1 is any one of K, R, and H
  misc_feature (6)..(6): Xaa at position 6 is D or E
XGLLLXY

SEQ ID NO: 61 - protein, 15 aa, Artificial sequence
  Sequence is synthesized
  misc_feature (1)..(1): Xaa at position 1 is any one of K, R, and H
  misc_feature (4)..(4): Xaa at position 4 is any one of K, R, and H
  misc_feature (5)..(5): Xaa at position 5 is T or S
  misc_feature (14)..(14): Xaa at position 14 is M or L
  misc_feature (15)..(15): Xaa at position 15 is any one of K, R, H, and A
XASXXVSTSSYSYXX

SEQ ID NO: 62 - protein, 7 aa, Artificial sequence
  Sequence is synthesized
  misc_feature (6)..(6): Xaa at position 6 is any one or D, E, N, and Q
YASYLXS

SEQ ID NO: 63 - protein, 9 aa, Artificial sequence
  Sequence is synthesized
  misc_feature (2)..(2): Xaa at position 1 is any one or K, R, and H
  misc_feature (4)..(4): Xaa at position 4 is any one of K, R, and H
  misc_feature (5)..(5): Xaa at position 5 is D or E
QXSXXFPWT

SEQ ID NO: 64 - protein, 116 aa, Artificial sequence
  Sequence is synthesized
EVQLVESGGGLVQPGGSLRLSCSASGFTFSNYGMHWVRQAPGKGLEYVSYISSGSSTIYYADTVKGRFTISRDNSKNTLYLQMSSLRAEDTAVYYCVKRGLLLDYWGQGTLVTVSS

SEQ ID NO: 65 - protein, 116 aa, Artificial sequence
  Sequence is synthesized
EVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 66 - protein, 116 aa, Artificial sequence
  Sequence is synthesized
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 67 - protein, 116 aa, Artificial sequence
  Sequence is synthesized
EVQLVQSGGGLIQPGGSLRLSCAASGFTFSNYGMHWVRQAPGKGLEWVSYISSGSSTIYYADSVKGRFTISRDNSKNTLYLQMNSLRAEDTAVYYCARRGLLLDYWGQGTTVTVSS

SEQ ID NO: 68 - protein, 116 aa, Artificial sequence
  Sequence is synthesized
EVQLVESGGGLIQPGGSLRLSCAASGFTFSNYGMHWVRQAPGKGLEWVSYISSGSSTIYYADSVKGRFTISRDNSKNTLYLQMTSLRAEDTAVYYCARRGLLLDYWGQGTTLTVSS

SEQ ID NO: 69 - protein, 116 aa, Artificial sequence
  Sequence is synthesized
EVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSS

SEQ ID NO: 70 - protein, 116 aa, Artificial sequence
  Sequence is synthesized
EVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 71 - protein, 116 aa, Artificial sequence
  Sequence is synthesized
EVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 72 - protein, 116 aa, Artificial sequence
  Sequence is synthesized
EVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 73 - protein, 116 aa, Artificial sequence
  Sequence is synthesized
EVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 74 - protein, 111 aa, Artificial sequence
  Sequence is synthesized
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGQGTKVEIK

SEQ ID NO: 75 - protein, 111 aa, Artificial sequence
  Sequence is synthesized
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIK

SEQ ID NO: 76 - protein, 111 aa, Artificial sequence
  Sequence is synthesized
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIK

SEQ ID NO: 77 - protein, 111 aa, Artificial sequence
  Sequence is synthesized
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIK

SEQ ID NO: 78 - protein, 111 aa, Artificial sequence
  Sequence is synthesized
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIK

SEQ ID NO: 79 - protein, 111 aa, Artificial sequence
  Sequence is synthesized
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIK

SEQ ID NO: 80 - protein, 111 aa, Artificial sequence
  Sequence is synthesized
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIK

SEQ ID NO: 81 - protein, 111 aa, Artificial sequence
  Sequence is synthesized
DIQMTQSPSSLSASVGDRVTITCRASKSVSTSSYSYLAWYQQKPEKAPKLLIKYASYLQSGVPSRFSGSGSGTDFTLTISSLQPEDFATYYCQHSREFPWTFGAGTKLELK

SEQ ID NO: 82 - protein, 111 aa, Artificial sequence
  Sequence is synthesized
DIQMTQSPSSLSASVGDRVTISCRASKSVSTSSYSYMHWYQQKPEKAPKLLIKYASYLQSGVPSRFSGSGSGTDFTLTISSLQPEDVATYYCQHSREFPWTFGAGTKLELK

SEQ ID NO: 83 - protein, 515 aa, Artificial sequence
  Sequence is synthesized
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 84 - protein, 515 aa, Artificial sequence
  Sequence is synthesized
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 85 - protein, 515 aa, Artificial sequence
  Sequence is synthesized
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 86 - protein, 515 aa, Artificial sequence
  Sequence is synthesized
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 87 - protein, 515 aa, Artificial sequence
  Sequence is synthesized
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 88 - protein, 515 aa, Artificial sequence
  Sequence is synthesized
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 89 - protein, 515 aa, Artificial sequence
  Sequence is synthesized
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 90 - protein, 515 aa, Artificial sequence
  Sequence is synthesized
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 91 - protein, 515 aa, Artificial sequence
  Sequence is synthesized
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 92 - protein, 515 aa, Artificial sequence
  Sequence is synthesized
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 93 - protein, 515 aa, Artificial sequence
  Sequence is synthesized
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 94 - protein, 515 aa, Artificial sequence
  Sequence is synthesized
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 95 - protein, 515 aa, Artificial sequence
  Sequence is synthesized
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 96 - protein, 515 aa, Artificial sequence
  Sequence is synthesized
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 97 - protein, 515 aa, Artificial sequence
  Sequence is synthesized
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 98 - protein, 515 aa, Artificial sequence
  Sequence is synthesized
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 99 - protein, 8 aa, Artificial sequence
  Sequence is synthesized
GFTFSDYG

SEQ ID NO: 100 - protein, 8 aa, Artificial sequence
  Sequence is synthesized
GFTFSNYG

SEQ ID NO: 101 - protein, 8 aa, Artificial sequence
  Sequence is synthesized
ISSGSSTI

SEQ ID NO: 102 - protein, 8 aa, Artificial sequence
  Sequence is synthesized
ISSSSSTI

SEQ ID NO: 103 - protein, 9 aa, Artificial sequence
  Sequence is synthesized
ARRGLLLDY

SEQ ID NO: 104 - protein, 10 aa, Artificial sequence
  Sequence is synthesized
KSVSTSSYSY

SEQ ID NO: 105 - protein, 10 aa, Artificial sequence
  Sequence is synthesized
KTVSTSSYSY

SEQ ID NO: 106 - protein, 3 aa, Artificial sequence
  Sequence is synthesized
YAS

SEQ ID NO: 107 - protein, 9 aa, Artificial sequence
  Sequence is synthesized
QHSREFPWT

SEQ ID NO: 108 - protein, 115 aa, Artificial sequence
  Sequence is synthesized
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVS

SEQ ID NO: 109 - protein, 132 aa, Artificial sequence
  Sequence is synthesized
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGS

SEQ ID NO: 110 - RNA, 720 nt, Artificial sequence
  Sequence is synthesized
auggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguaa

SEQ ID NO: 111 - RNA, 89 nt, Artificial sequence
  Sequence is synthesized
ggagcaaaagcagggugacaaagacauaauggauccaaacacugugucaagcuuucagguagauugcuuucuuuggcauguccgcaaac

SEQ ID NO: 112 - RNA, 19 nt, Artificial sequence
  Sequence is synthesized
gcaugcgaccucuguuuga

SEQ ID NO: 113 - RNA, 19 nt, Artificial sequence
  Sequence is synthesized
ucaaacagaggucgcaugc

(19) United States
(12) Patent Application Publication
(10) Pub. No.: US 2024/0181086 A1
(43) Pub. Date: Jun. 6, 2024

(54) COMPOSITIONS AND METHODS FOR DELIVERING THERAPEUTIC POLYNUCLEOTIDES

(71) Applicants: Yale University, New Haven, CT (US); Gennao Bio, Inc., Pennington, NJ (US)

(72) Inventors: Elias Quijano, Durham, CT (US); Peter Glazer, New Haven, CT (US); Bruce C. Turner, Bryn Mawr, PA (US); Stephen Squinto, New York, NY (US)

(21) Appl. No.: 18/548,827

(22) PCT Filed: Mar. 3, 2022
(86) PCT No.: PCT/US2022/018789

§ 371 Date: Sep. 1, 2023

Related U.S. Application Data
(60) Provisional application No. 63/156,070, filed on Mar. 3, 2021.

(51) Int. Cl. A61K 48/00; A61K 39/00; C07K 16/44; C12N 15/87

(52) U.S. Cl. CPC A61K 48/0066; C07K 16/44; C12N 15/87; A61K 2039/505; C07K 2317/565; C07K 2317/77

(57) ABSTRACT

Compositions and methods are provided for delivering therapeutic polynucleotides by administering a complex formed between a therapeutic polynucleotide having a 3E10 or 3E10 variant binding domain and a 3E10 antibody or variant thereof, or antigen-binding fragment thereof. In some instances, the complexes are stabilized through a molar ratio of 3E10 antibody or variant thereof, or antigen-binding fragment thereof to therapeutic polynucleotide of at least about 2:1.

### CROSS REFERENCE TO RELATED APPLICATIONS

This application claims priority to U.S. Provisional Patent Application No. 63/156,070, filed Mar. 3, 2021, the contents of which are hereby incorporated by reference in their entireties for all purposes.

### STATEMENT REGARDING FEDERALLY SPONSORED RESEARCH

This invention was made with government support under R35CA197574 awarded by National Institutes of Health. The Government has certain rights in the invention.

### TECHNICAL FIELD

The present disclosure relates generally to compositions and methods for delivering therapeutic polynucleotides having preferred 3E10 binding sequences.

### BACKGROUND

Therapeutic polynucleotide-based therapies offer promising options for treatment of disease conditions of varying etiology. Polynucleotide therapies include therapies that deliver a gene or transcript encoding a functional version of a protein affected by a genetic disorder, therapies that direct transcriptional/translational down-regulation such as siRNA therapies, and therapies that deliver polynucleotides that direct editing of the subject's genome. In fact, several polynucleotide therapies have won regulatory approval and numerous others are currently in clinical trials for the treatment of various disease conditions.

However, nucleic acid-based therapies are limited by the need for improved delivery systems. For instance, naked polynucleotides are readily degraded by a host of extracellular nucleases present in skin, tissues, and blood. Further, naked polynucleotides do not readily cross the cell membrane. Conventional approaches to overcoming these obstacles include packaging therapeutic polynucleotides into liposomal-based delivery vehicles or recombinant viral particles. However, both of these strategies present immunological challenges, because the viral capsids and liposomal vehicles are recognized by the host's immune system, and can have limitations in delivery efficiency.

### SUMMARY

Given the background above, improved methods are needed for delivering therapeutic polynucleotides in vivo. Advantageously, the present disclosure provides compositions and methods for delivering therapeutic polynucleotides in vivo that are not reliant upon liposomal or viral vector based nucleic acid delivery. In some aspects, these compositions and methods are based on, at least in part, on the discovery of 3E10 and 3E10 variant nucleotide binding preferences, and use of these preferences to provide molecular handles that improve binding between 3E10 and 3E10 variants and therapeutic polynucleotides.

In some embodiments, the advantageous properties of the compositions and methods described herein are based, at least in part, on the discovery that 3E10 antibodies or variants thereof, or antigen-binding fragments thereof, as described below, bind to certain single-stranded polynucleotide sequences more strongly than other single-stranded and double-stranded polynucleotide sequences. For instance, as described in Example 7, 3E10 (D31N) variants preferentially bind to poly-dT (FIG. 14D), relative to the other three poly-deoxyribonucleotides (FIGS. 14A-14C). Further, both 3E10 (D3IN) and 3E10 bind poly-dA (FIG. 14C) with lower affinity than the other three poly-deoxyribonucleotides (FIGS. 14A-14B and 14D). Similarly, as described in Example 8, 3E10 (D31N) variants preferentially bind to poly-rl and poly-rG, relative to the other poly-ribonucleotides (FIG. 15K). Advantageously, the identified nucleotide binding preferences are exploited in the compositions and methods described herein to provide nucleotide sequences-nucleotide handles, sites, or domains—that can be integrated into therapeutic polynucleotides in order to improve binding between the 3E10 antibodies or variants thereof, or antigen-binding fragments thereof and the therapeutic polynucleotides.

In some embodiments, the advantageous properties of the compositions and methods described herein are based, at least in part, on the discovery that 3E10 antibodies or variants thereof, or antigen-binding fragments thereof, as described below, localize to skeletal muscle tissue in vivo (in relation to other tissues or organs) following systemic or intramuscular administration. For instance, as described in Example 3 and illustrated in FIG. 10, following intravenous administration, both parental 3E10 antibody and 3E10 (D31N) variant antibody accumulated at greater concentrations in skeletal muscle than in other non-hepatic tissues, e.g., brain, lung, heart, spleen, and renal tissues. Advantageously, this tropism for skeletal muscle tissue is exploited in the compositions and methods described herein to deliver therapeutic polynucleotides to skeletal muscle tissue for treatment of various skeletal muscle disorders.

In some embodiments, the advantageous properties of the compositions and methods described herein are based, at least in part, on the discovery that use of higher molar ratios of 3E10 antibody or variant thereof, or antigen-binding fragment thereof to therapeutic polynucleotide result in greater protection of the therapeutic polynucleotide from degradation. For instance, as described in Example 6 and illustrated in FIGS. 13A and 13B, while parental 3E10 and 3E10 (D31N) variant antibodies protected mRNA from RNAse A-mediated RNA degradation at molar ratios of 2:1 and 20:1, the protection afforded by the 20:1 molar ratio exceeded the protection afforded at 2:1. Advantageously, the increased therapeutic polynucleotide protection at higher 3E10 antibody or variant thereof, or antigen-binding fragment thereof concentrations is exploited in the compositions and methods described herein to improve the pharmacokinetic properties of therapeutic compositions delivering therapeutic polynucleotides in vivo.

In some embodiments, the advantageous properties of the compositions and methods described herein are based, at least in part, on the discovery that sustained protein expression in skeletal muscle tissue from a therapeutic mRNA is realized by administration of a complex of the 3E10 antibody or variant thereof, or antigen-binding fragment thereof and the therapeutic mRNA. For instance, as described in Example 5 and illustrated in FIGS. 12A-12B, intramuscular administration of a 3E10 (D31N) variant antibody-mRNA complex resulted in sustained expression of a luciferase encoded by the mRNA for at least five days. Advantageously, the sustained expression in skeletal muscle tissue resulting from administration of these complexes is exploited in the compositions and methods described herein to treat skeletal muscle disorders with long-acting compositions.

Accordingly, one aspect of the present disclosure provides a pharmaceutical composition including a therapeutically effective amount of a complex formed between a therapeutic polynucleotide having a 3E10 or 3E10 variant binding domain that is preferably bound by a 3E10 or 3E10 variant antibody, and a 3E10 antibody or variant thereof, or antigen-binding fragment thereof.

In another aspect, the disclosure provides a pharmaceutical composition including a therapeutically effective amount of a complex formed between a therapeutic polynucleotide, and a 3E10 antibody or variant thereof, or antigen-binding fragment thereof, where the therapeutic polynucleotide has a first codon-altered nucleotide sequence encoding a therapeutic polypeptide, and the 3E10 antibody or variant thereof, or antigen-binding fragment thereof has a greater affinity for the first codon-altered nucleotide sequence than for a second nucleotide sequence that encodes the therapeutic polypeptide using a same coding sequence for the therapeutic polypeptide as found in a genome for the species of the subject.

In another aspect, the disclosure provides a method for delivering a therapeutic polynucleotide to a tissue of a subject in vivo, the method including parenterally administering a pharmaceutical composition, as described herein, to the subject. In some embodiments, the therapeutic polynucleotide is for treating a skeletal muscle disorder.

In some embodiments of the methods and compositions described herein, the 3E10 antibody or variant thereof, or antigen-binding fragment thereof includes (a) a light chain variable region (VL) complementarity determining region (CDR) 1 comprising the amino acid sequence of 3E10-VL-CDR1 (SEQ ID NO:9), (b) a VL CDR2 comprising the amino acid sequence of 3E10-VL-CDR2 (SEQ ID NO:10), (c) a VL CDR3 comprising the amino acid sequence of 3E10-VL-CDR3 (SEQ ID NO:11), (d) a heavy chain variable region (VH) CDR1 comprising the amino acid sequence of 3E10-VH-CDR1a (SEQ ID NO: 16), (e) a VH CDR2 comprising the amino acid sequence of 3E10-VH-CDR2 (SEQ ID NO:4), and (f) a VH CDR3 comprising the amino acid sequence of 3E10-VH-CDR3 (SEQ ID NO:5).

In some embodiments of the methods and compositions described herein, the 3E10 antibody or variant thereof, or antigen-binding fragment thereof includes (a) a light chain variable region (VL) complementarity determining region (CDR) 1 comprising an amino acid sequence having no more than two amino acid substitutions relative to 3E10-VL-CDR1 (SEQ ID NO:9), (b) a VL CDR2 comprising an amino acid sequence having no more than two amino acid substitutions relative to 3E10-VL-CDR2 (SEQ ID NO:10), (c) a VL CDR3 comprising an amino acid sequence having no more than two amino acid substitutions relative to 3E10-VL-CDR3 (SEQ ID NO:11), (d) a heavy chain variable region (VH) CDR1 comprising an amino acid sequence having no more than two amino acid substitutions relative to 3E10-VH-CDR1a (SEQ ID NO:16), (e) a VH CDR2 comprising an amino acid sequence having no more than two amino acid substitutions relative to 3E10-VH-CDR2 (SEQ ID NO:4), and (f) a VH CDR3 comprising an amino acid sequence having no more than two amino acid substitutions relative to 3E10-VH-CDR3 (SEQ ID NO:5).

In some embodiments of the methods and compositions described herein, the 3E10 antibody or variant thereof, or antigen-binding fragment thereof includes (a) a light chain variable region (VL) complementarity determining region (CDR) 1 comprising the amino acid sequence of 3E10-VL-CDR1m (SEQ ID NO:61), (b) a VL CDR2 comprising the amino acid sequence of 3E10-VL-CDR2m (SEQ ID NO:62), (c) a VL CDR3 comprising the amino acid sequence of 3E10-VL-CDR3m (SEQ ID NO:63), (d) a heavy chain variable region (VH) CDR1 comprising the amino acid sequence of 3E10-VH-CDR1m (SEQ ID NO:58), (e) a VH CDR2 comprising the amino acid sequence of 3E10-VH-CDR2m (SEQ ID NO:59), and (f) a VH CDR3 comprising the amino acid sequence of 3E10-VH-CDR3m (SEQ ID NO:60).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates amino acid sequences for the parent 3E10 monoclonal antibody.

FIGS. 2A, 2B, and 2C illustrate amino acid sequences for the D3IN variant (FIG. 2A), other CDR variants (FIG. 2B), and additionally contemplated CDR variants (FIG. 2C) of the 3E10 monoclonal antibody, in accordance with some embodiments of the present disclosure.

FIG. 3 illustrates example charge-conserved CDR variants of the 3E10 monoclonal antibody, in accordance with various embodiments of the present disclosure.

FIG. 4 illustrates example CDR variants containing a combination of amino acid substitutions, charged-conserved amino acid substitutions, and rationally-designed amino acid substitutions of the 3E10 monoclonal antibody, in accordance with various embodiments of the present disclosure.

FIG. 5 illustrates a sequence alignment of examples of humanized 3E10 heavy chain variable regions, with CDRs underlined as indicated.

FIG. 6 illustrates a sequence alignment of examples of humanized 3E10 light chain variable regions, with CDRs and putative nuclear localization signals (NLS) underlined as indicated.

FIGS. 7A, 7B, 7C, 7D, and 7E collectively illustrate a sequence alignment of example of humanized di-scFv constructs of the 3E10 monoclonal antibody.

FIG. 8 illustrates a line graph showing 3E10-mediated delivery of mRNA (bioluminescene (Photons/second)) to mouse muscles (IM) over time (days post-IM injection), in accordance with some embodiments of the present disclosure.

FIGS. 9A, 9B, and 9C collectively show fluorescently-labeled 3E10 (D31N) antibody localization in mouse skeletal muscle following intravenous administration. FIGS. 9A and 9B are images of fluorescence in mouse skeletal muscle following intravenous injections of a control composition (FIG. 9A) or fluorescently-labeled 3E10 (D31N) antibody (FIG. 9B), acquired by IVIS (Perkin Elmer) 24 hours after administration. FIG. 9C is a bar graph quantifying the fluorescence in the IVIS images.

FIG. 10 is a bar graph quantifying the fluorescence in IVIS images of dose-dependent biodistribution of 3E10-D31N to tissues 24 hours following 100 μg or 200 μg intravenous injection of 3E10-D31N labeled with VivoTag680 into mice (Perkin Elmer).

FIGS. 11A and 11B illustrate electrostatic surface potential renderings of a molecular model of a 3E10-scFv construct, revealing a putative Nucleic Acid Binding pocket (NAB1).

FIG. 11A additionally shows predicted structural and electrostatic potential changes induced by amino acid substitutions at residue HC CDR1 residue 31. FIG. 11B is an illustration of molecular modeling of 3E10-scFv (Pymol) with NAB1 amino acid residues highlighted by punctate dots.

FIG. 11C illustrates mapping of the putative nucleic acid binding pocket, as identified by the molecular modeling shown in FIGS. 11A and 11B, onto the amino acid sequence of the 3E10-scFv construct.

FIGS. 12A and 12B show expression of mRNA in skeletal muscle following intramuscular administration of a 3E10 (D31N)-mRNA construct. FIG. 12A show fluorescent images of a mouse over a five-day time course following intramuscular administration of mRNA encoding a luciferase complexed with 3E10 (D31N). FIG. 12B illustrates a bar graph quantifying average radiance over all pixels, showing fluorescence in single mice in images of control mice (untreated) and mice administered the 3E10 (D31N)-mRNA construct intramuscularly.

FIGS. 13A and 13B show gel electrophoresis analysis of mRNA protection assays performed with 3E10 (D31N)-mRNA constructs prepared at 20:1 (FIG. 13A) and 2:1 (FIG. 13B) molar ratios.

FIGS. 14A, 14B, 14C, and 14D illustrate results of 3E10 and 3E10 (D31N) binding to different species of poly-deoxyribonucleotides.

FIGS. 15A, 15B, 15C, 15D, 15E, 15F, 15G, 15H, 15I, 151, 15J, 15K, and 15L illustrate results of 3E10 and 3E10 (D31N) binding to different species of poly-deoxyribonucleotides and poly-ribonucleotides.

FIG. 16 is a bar graph showing 3E10-mediated delivery and stimulation of RIG-I.

FIGS. 17A, 17B, 17C, 17D, 17E, and 17F illustrate example architectures of therapeutic polynucleotides with a 3E10 or 3E10 variant binding domain, in accordance with some embodiments of the present disclosure.

FIG. 18 illustrates an example codon-skewed therapeutic polynucleotide, in accordance with some embodiments of the present disclosure.

FIG. 19 illustrates results of 3E10 and 3E10 (D31N) binding to different species of poly-deoxyribonucleotides.

FIGS. 20A, 20B, 20C, and 20D illustrate results of 3E10 and various 3E10 variant binding to different species of poly-deoxyribonucleotides.

FIG. 21 shows gel electrophoresis analysis of mRNA protection assays performed with 3E10 and 3E10 (D31N)-mRNA constructs prepared at 20:1 molar ratios.

FIG. 22 shows a histogram of cytosolic, membrane, nuclear protein, and gDNA fractions after administration of ⁸⁹Zr labeled isotype control, 3E10-WT, and 3E10-D31N antibodies, as described in Example 13.

FIG. 23 shows gel electrophoresis analysis of mRNA protection assays performed with complexes formed between 3E10 and a 14 kb mRNA encoding the human dystrophin protein, prepared at 1:1, 2:1, 5:1, 10:1 and 100:1 (3E10:mRNA) molar ratios, as described in Example 14.

### DETAILED DESCRIPTION

The present disclosure provides compositions and methods for delivering therapeutic polynucleotides, in vivo, that are not reliant upon the conventional viral-based or liposomal-based delivery methodologies associated with difficult and costly production, limited packaging capacity, and adverse immunological events. In some aspects, described in greater detail below, these compositions and methods are based on, at least in part, on the discovery of nucleotide binding preferences for 3E10 antibodies or variants thereof, or antigen-binding fragments thereof. These preferences can be used, for example, to design polynucleotide sequences that can be incorporated into therapeutic polynucleotides to improve binding between the therapeutic polynucleotides and an 3E10 antibody or variant thereof, or antigen-binding fragment thereof, thus increasing the in vivo effectiveness of these complexes.

Further, it was discovered that 3E10 antibodies or variants thereof, or antigen-binding fragments thereof help transport polynucleotides across the plasma membrane, into the cell cytoplasm and/or nucleus. Thus, compositions and methods for using 3E10 antibodies or variants thereof, or antigen-binding fragments thereof to enhance delivery of therapeutic polynucleotides, particularly to skeletal muscle tissue, are provided.

### Definitions.

The terminology used in the present disclosure is for the purpose of describing particular embodiments only and is not intended to be limiting of the invention. As used in the description of the invention and the attached claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. Unless the context requires otherwise, it will be further understood that the terms “includes,” “comprising,” or any variation thereof, when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%.

By “antigen binding domain” or “ABD” herein is meant a set of six Complementary Determining Regions (CDRs) that, when present as part of a polypeptide sequence or sequences, specifically binds a target antigen as discussed herein. Thus, a “nucleic acid binding domain” binds a nucleic acid antigen as outlined herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 for the heavy chain and vlCDR1, vICDR2 and vICDR3 for the light. The CDRs are present in the variable heavy and variable light domains, respectively, and together form an Fv region. (See Table 1 and related discussion above for CDR numbering schemes). Thus, in some cases, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vICDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used. In general, the C-terminus of the scFv domain is attached to the N-terminus of the hinge in the second monomer.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g. vICDR1, vICDR2 and vICDR3). A useful comparison of CDR numbering is as below, see Lafranc et al., Dev. Comp. Immunol. 27(1):55-77 (2003):

TABLE 1

| | Kabat + Chothia | IMGT | Kabat | AbM | Chothia | Contact | Xencor |
|---|---|---|---|---|---|---|---|
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g, Kabat et al., supra (1991)). The EU index or EU index as in Kabat or

EU numbering scheme refers to the numbering of the EU antibody. Kabat et al. collected numerous primary sequences of the variable regions of heavy chains and light chains. Based on the degree of conservation of the sequences, they classified individual primary sequences into the CDR and the framework and made a list thereof. See, SEQUENCES OF IMMUNOLOGICAL INTEREST, 5th edition, NIH publication, No. 91-3242, E. A. Kabat et al.; Edelman et al., 1969, Proc Natl Acad Sci USA 63:78-85, the contents of which are incorporated herein by reference. The modification can be an addition, deletion, or substitution.

By “target antigen” as used herein is meant the molecule that is bound specifically by the antigen binding domain comprising the variable regions of a given antibody. As discussed below, in the present case the target antigens are nucleic acids.

As described below, in some embodiments a parent polypeptide, for example an Fc parent polypeptide, is a human wild type sequence, such as the heavy constant domain or Fc region from IgG1, IgG2, IgG3 or IgG4, although human sequences with variants can also serve as “parent polypeptides”, for example the IgG1/2 hybrid of US Publication 2006/0134105 (which is hereby incorporated by reference) can be employed. The protein variant sequence herein will preferably possess at least about 75% identity with a parent protein sequence, or at least about 80% identity with a parent protein sequence, and most preferably at least about 90% identity, more preferably at least about 95%, or at least about 98%, or at least about 99% sequence identity. In some embodiments, the protein variant sequence herein has at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity with a parent protein sequence. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2, IgG3, or IgG4.

By “isotype” as used herein is meant any of the subclasses of immunoglobulins defined by the chemical and antigenic characteristics of their constant regions. It should be understood that therapeutic antibodies can also comprise hybrids of isotypes and/or subclasses.

By “Fab” or “Fab region” as used herein is meant a polypeptide that comprises the VH, CH1, VL, and CL immunoglobulin domains, generally on two different polypeptide chains (e.g. VH-CH1 on one chain and VL-CL on the other). Fab may refer to this region in isolation, or this region in the context of an antibody of the disclosure. In the context of a Fab, the Fab comprises an Fv region in addition to the CH1 and CL domains.

By “Fv” or “Fv fragment” or “Fv region” as used herein is meant a polypeptide that comprises the VL and VH domains of an ABD. Fv regions can be formatted as both Fabs (as discussed above, generally two different polypeptides that also include the constant regions as outlined above) and scFvs, where the vl and vh domains are combined (generally with a linker as discussed herein) to form an scFv.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g. H.X_L. Y means N- to C-terminal is vh-linker-vl, and L. Y H.X is vl-linker-vh.

By “Fc” or “Fc region” or “Fc domain” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus the definition of “Fc domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g. non-denaturing chromatography, size exclusion chromatography, etc.) Human IgG Fc domains are of particular use in the present disclosure, and can be the Fc domain from human IgG1, IgG2 or IgG4.

A “variant Fc domain” contains amino acid modifications as compared to a parental Fc domain. Thus, a “variant human IgG1 Fc domain” is one that contains amino acid modifications (generally amino acid substitutions, although in the case of ablation variants, amino acid deletions are included) as compared to the human IgG1 Fc domain. In general, variant Fc domains have at least about 80, about 85, about 90, about 95, about 97, about 98 or about 99 percent identity to the corresponding parental human IgG Fc domain (using the identity algorithms discussed below, with one embodiment utilizing the BLAST algorithm as is known in the art, using default parameters). Alternatively, the variant Fc domains can have from 1 to about 20 (e.g., 1, 2, 3, 4, 5, 6, 7, 8, 9, 10, 11, 12, 13, 14, 15, 16, 17, 18, 19 or 20) amino acid modifications as compared to the parental Fc domain. Additionally, as discussed herein, the variant Fc domains herein still retain the ability to form a dimer with another Fc domain as measured using known techniques as described herein, such as non-denaturing gel electrophoresis.

By “heavy chain constant region” herein is meant the CH1-hinge-CH2-CH3 portion of an antibody (or fragments thereof), excluding the variable heavy domain; in EU numbering of human IgG1 this is amino acids 118-447 By “heavy chain constant region fragment” herein is meant a heavy chain constant region that contains fewer amino acids from either or both of the N- and C-termini but still retains the ability to form a dimer with another heavy chain constant region.

By “variable region” or “variable domain” as used herein is meant the region of an immunoglobulin that comprises one or more Ig domains substantially encoded by any of the VK, VÀ, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vICDR2 and vlCDR3 for the variable light domain) and four framework (FR) regions, arranged from amino-terminus to carboxy-terminus in the following order: FR1-CDR1-FR2-CDR2-FR3-CDR3-FR4.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

The antibodies of the present disclosure are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogenous host cells, and they can be isolated as well.

As used herein, the term “cell-penetrating antibody” refers to an immunoglobulin protein, fragment, variant thereof, or fusion protein based thereon that is transported into the cytoplasm and/or nucleus of living mammalian cells. The “cell-penetrating anti-DNA antibody” specifically binds DNA (e.g., single-stranded and/or double-stranded DNA). In some embodiments, the antibody is transported into the cytoplasm of the cells without the aid of a carrier or conjugate. In other embodiments, the antibody is conjugated to a cell-penetrating moiety, such as a cell penetrating peptide. In some embodiments, the cell-penetrating antibody is transported in the nucleus with or without a carrier or conjugate.

By “skeletal muscle polypeptide” or “skeletal muscle protein” herein is meant a polypeptide having a substantially similar structure and function as a protein, or polypeptide chain thereof, that is genetically-linked to a skeletal muscle disorder, such as a myopathy, e.g., a protein, or polypeptide chain thereof, for which mutations exist that result in a skeletal muscle disorder. The term “skeletal muscle polypeptide” encompasses wild type versions of skeletal muscle proteins, and polypeptide chains thereof, natural variant versions of skeletal muscle proteins, and polypeptide chains thereof, as well as engineered versions of skeletal muscle proteins, and polypeptide chains thereof. Skeletal muscle polypeptides are also intended to encompass proteins, and polypeptide chains thereof, having a function that partially or completely rescues a function lost by a mutation in a protein, or polypeptide chain thereof, genetically-linked to a skeletal muscle disorder, including but not limited to various homologues of a skeletal muscle protein, or polypeptide chain thereof.

By “modification” herein is meant an amino acid substitution, insertion, and/or deletion in a polypeptide sequence.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the parent protein, using the alignment programs described below, such as BLAST.

Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol.48:443 [homology alignment algorithm], Pearson, W.R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see the webpage located at URL blast.ncbi.nlm.nih.gov/Blast.cgi. When using any of the aforementioned algorithms, the default parameters (for Window length, gap penalty, etc.) are used. Unless specifically stated otherwise, sequence identity is determined using the BLAST algorithm, using default parameters

As used herein, the term “subject” means any individual who is the target of administration. The subject can be a vertebrate, for example, a mammal. Thus, the subject can be a human. The term does not denote a particular age or sex.

As used herein, the term “pharmaceutically effective amount” means that the amount of the composition used is of sufficient quantity to ameliorate one or more causes or symptoms of a disease or disorder. Such amelioration only requires a reduction or alteration, not necessarily elimination. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “carrier” or “excipient” refers to an organic or inorganic ingredient, natural or synthetic inactive ingredient in a formulation, with which one or more active ingredients are combined. The carrier or excipient would naturally be selected to minimize degradation of the active ingredient or to minimize adverse side effects in the subject, as would be well known to one of skill in the art.

As used herein, the term “treat” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “genetic skeletal muscle disorder” refers to a disorder having a genetic basis that primarily affects the skeletal muscle tissue. Genetic skeletal muscle disorders are caused by mutations in various genes encoding proteins that function in muscle structure and function. Genetic skeletal muscle disorders typically manifest as skeletal muscle weakness and hypotonia. Non-limiting examples of different types of genetic skeletal muscle disorders are provided in Table 2.

### 3E10 Antibodies, Variants, and Fragments Thereof

In some aspects, the present disclosure relates to the use of 3E10 antibodies, and derivatives thereof, for delivering therapeutic polynucleotides to tissues in a subject, including but not limited to skeletal muscle tissues for treatment of genetic skeletal muscle disorders. As is discussed below, the term antibody is used generally. Antibodies that find use in the present disclosure take on a number of formats as described herein, including traditional antibodies as well as antibody derivatives, fragments, and mimetics, described herein in various embodiments.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present disclosure is directed to antibodies that generally are based on the IgG class, which has several subclasses, including, but not limited to IgG1, IgG2, IgG3, and IgG4. In general, IgG1, IgG2 and IgG4 are used more frequently than IgG3. It should be noted that IgG1 has different allotypes with polymorphisms at 356 (D or E) and 358 (L or M).

The light chain generally comprises two domains, the variable light domain (containing the light chain CDRs and together with the variable heavy domains forming the Fv region), and a constant light chain region (often referred to as CL or CK). The heavy chain comprises a variable heavy domain and a constant domain, which includes a CH1-optional hinge-Fc domain comprising a CH2-CH3.

The hypervariable region of an antibody generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs useful for the compositions and methods described herein are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vICDR1, vlCDR2 and vICDR3). A useful comparison of CDR numbering is described in Lafranc et al., Dev. Comp. Immunol. 27(1):55-77 (2003).

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The present disclosure provides a large number of different CDR sets. In this case, a “full CDR set” comprises the three variable light and three variable heavy CDRs, e.g. a vlCDR1, VICDR2, vICDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as nucleic acids, amino acids, or sugar side chains and usually have specific structural characteristics, as well as specific charge characteristics. A single antigen may have more than one epitope. The antibodies described herein bind to nucleic acid epitopes in a partially sequence-independent manner. That is, while the antibodies described herein bind to some polynucleotide structures and sequences with greater affinity than other nucleic acid structures and sequences, they have some general affinity for polynucleotides.

The “Fc domain” of the heavy chain includes the —CH2—CH3 domain, and optionally a hinge domain (—H—CH2—CH3). For IgG, the Fc domain comprises immunoglobulin domains CH2 and CH3 (Cγ2 and Cγ3) and the lower hinge region between CH1 (Cγ1) and CH2 (Cγ2). Although the boundaries of the Fc region may vary, the human IgG heavy chain Fc region is usually defined to include residues C226 or P230 to its carboxyl-terminus, wherein the numbering is according to the EU index as in Kabat. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. Thus, the “Fc domain” includes the —CH2—CH3 domain, and optionally a hinge domain (hinge-CH2—CH3). In the embodiments herein, when a scFv is attached to an Fc domain, it is generally the C-terminus of the scFv construct that is attached to all or part of the hinge of the Fc domain; for example, it is generally attached to the sequence EPKS which is the beginning of the hinge. In some embodiments, as is more fully described below, amino acid modifications are made to the Fc region, for example to alter binding to one or more FcγR receptors or to the FcRn receptor, and to enable heterodimer formation and purification, as outlined herein.

Another part of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (p230 in IgG1), wherein the numbering is according to the EU index as in Kabat. In some cases, a “hinge fragment” is used, which contains fewer amino acids at either or both of the N- and C-termini of the hinge domain.

A scFv comprises a variable heavy chain, an scFv linker, and a variable light domain. In most of the constructs and sequences outlined herein, the C-terminus of the variable heavy chain is attached to the N-terminus of the scFv linker, the C-terminus of which is attached to the N-terminus of a variable light chain (N-vh-linker-vl-C) although that can be switched (N-vl-linker-vh-C).

Thus, the present disclosure relates to different antibody domains. These domains include, but are not limited to, the Fc domain, the CH1 domain, the CH2 domain, the CH3 domain, the hinge domain, the heavy constant domain (CH1-hinge-Fc domain or CH1-hinge-CH2—CH3), the variable heavy domain, the variable light domain, the light constant domain, Fab domains and scFv domains.

In certain embodiments, the antibodies of the disclosure comprise a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence, e.g., that of the 3E10 antibody. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene. In certain cases, the humanized antibody may display no more than 5, or even no more than 4, 3, 2, or 1 amino acid difference from the amino acid sequence encoded by the germline immunoglobulin gene.

In one embodiment, the parent antibody has been affinity matured, as is known in the art. Structure-based methods may be employed for humanization and affinity maturation, for example as described in U.S. Ser. No. 11/004,590 (issued as U.S. Pat. No. 7,657,380), which is incorporated herein by reference. Selection based methods may be employed to humanize and/or affinity mature antibody variable regions, including but not limited to methods described in Wu et al., 1999, J. Mol. Biol. 294: 151-162; Baca et al., 1997, J. Biol. Chem. 272(16): 10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all of which are incorporated herein by reference. Other humanization methods may involve the grafting of only parts of the CDRs, including but not limited to methods described in U.S. Ser. No. 09/810,510 (published as US 2001/0035606); Tan et al., 2002, J. Immunol. 169:1119-1125; De Pascalis et al., 2002, J. Immunol. 169:3076-3084, all of which are incorporated herein by reference.

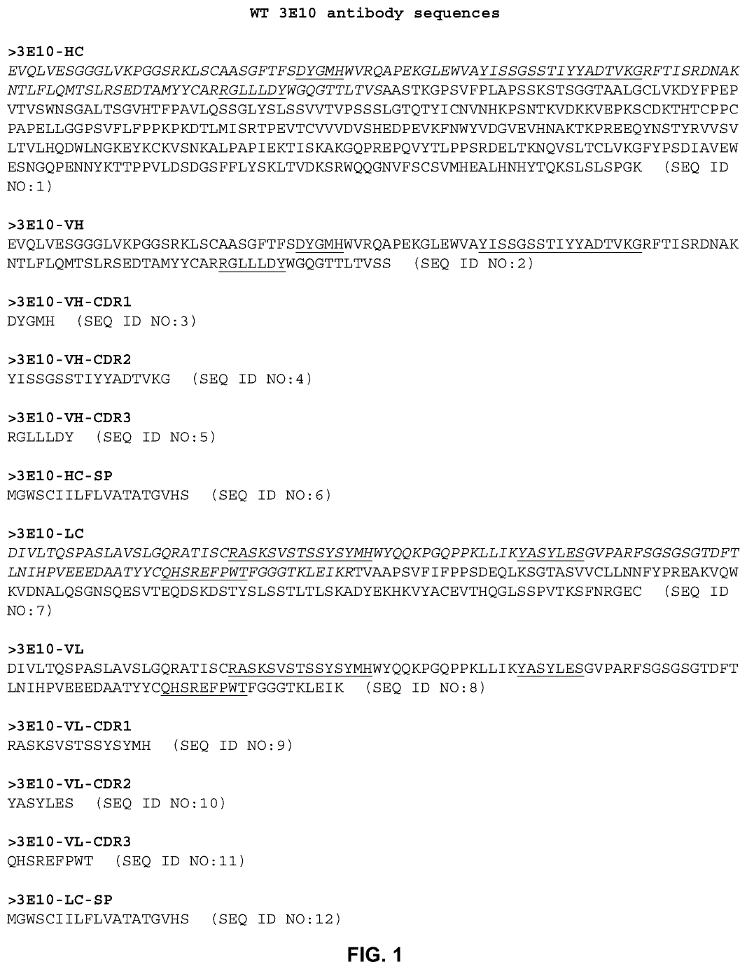

In some aspects, the disclosure relates to the use of antigen binding domains (ABDs) that bind to nucleic acids, and specifically that bind to therapeutic polynucleotides, derived from the 3E10 antibody. The amino acid sequence of the heavy and light chains of the parent 3E10 antibody are shown in FIG. 1. Accordingly, in some embodiments, the compositions described herein include a 3E10 antibody or variant thereof, or antigen-binding fragment thereof.

In some embodiments, a 3E10 antibody or variant thereof, or antigen-binding fragment thereof described herein includes CDR sequences corresponding to the parent 3E10 antibody, shown in FIG. 1. Accordingly, in some embodiments, the a 3E10 antibody or variant thereof, or antigen-binding fragment thereof includes a light chain variable region (VL) complementarity determining region (CDR) 1 comprising the amino acid sequence of 3E10-VL-CDR1 (SEQ ID NO:9), a VL CDR2 comprising the amino acid sequence of 3E10-VL-CDR2 (SEQ ID NO:10), a VL CDR3 comprising the amino acid sequence of 3E10-VL-CDR3 (SEQ ID NO:11), a heavy chain variable region (VH) CDR1 comprising the amino acid sequence of 3E10-VH-CDR1 (SEQ ID NO:3), a VH CDR2 comprising the amino acid sequence of 3E10-VH-CDR2 (SEQ ID NO:4), and a VH CDR3 comprising the amino acid sequence of 3E10-VH-CDR3 (SEQ ID NO:5).

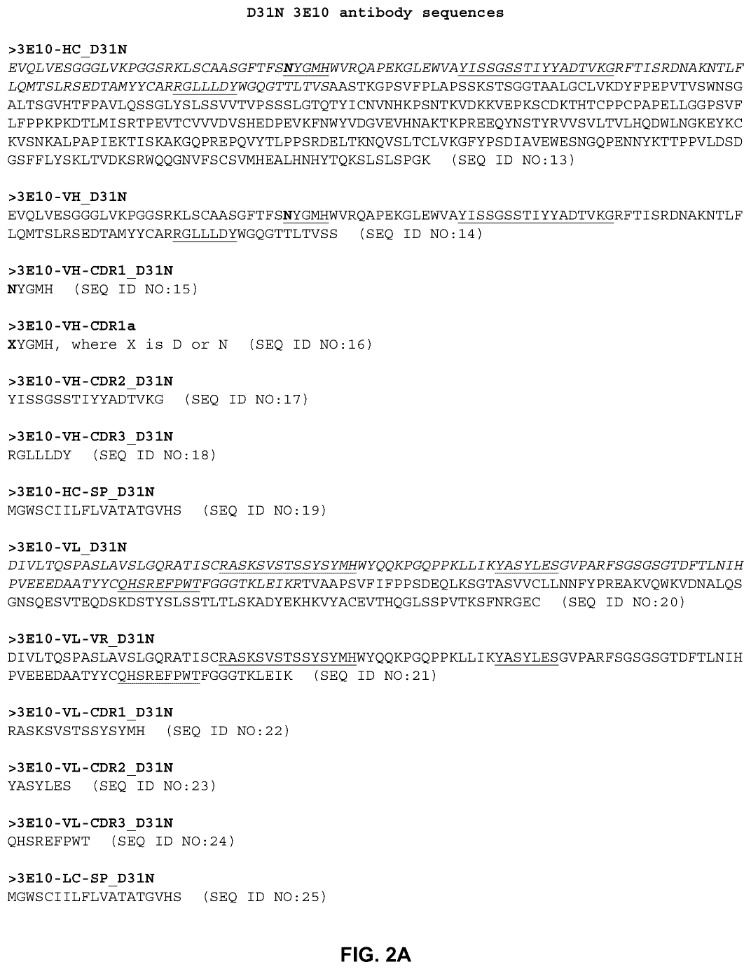

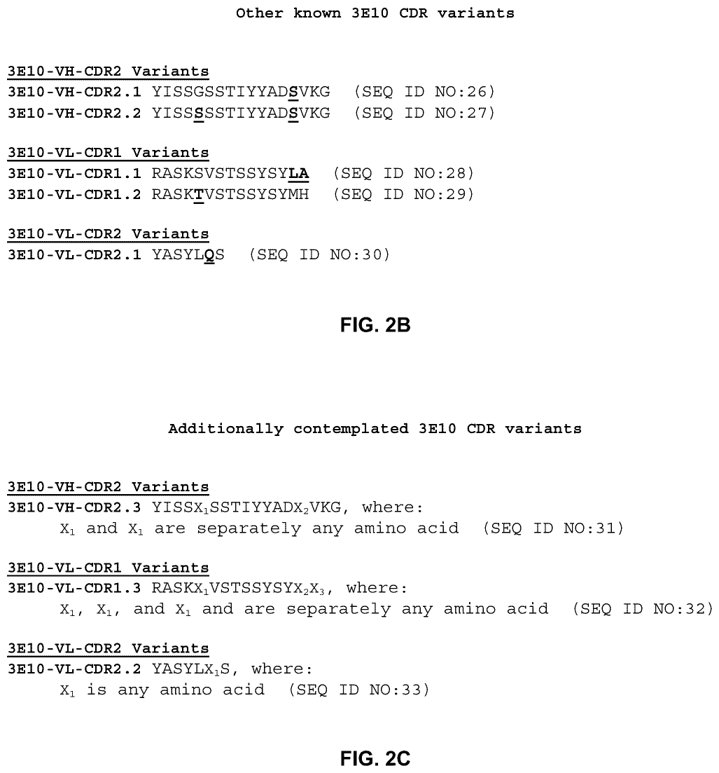

In some embodiments, a 3E10 antibody or variant thereof, or antigen-binding fragment thereof described herein includes CDR sequences from a variant 3E10 antibody that includes a D3IN amino acid substitution in the VH CDR1, as shown in FIG. 2. Accordingly, in some embodiments, the a 3E10 antibody or variant thereof, or antigen-binding fragment thereof includes a light chain variable region (VL) complementarity determining region (CDR) 1 comprising the amino acid sequence of 3E10-VL-CDR1_D3IN (SEQ ID NO:22), a VL CDR2 comprising the amino acid sequence of 3E10-VL-CDR2_D3IN (SEQ ID NO:23), a VL CDR3 comprising the amino acid sequence of 3E10-VL-CDR3_D3IN (SEQ ID NO:24), a heavy chain variable region (VH) CDR1 comprising the amino acid sequence of 3E10-VH-CDR1 D3IN (SEQ ID NO: 15), a VH CDR2 comprising the amino acid sequence of 3E10-VH-CDR2 D3IN (SEQ ID NO:17), and a VH CDR3 comprising the amino acid sequence of 3E10-VH-CDR3_D31N (SEQ ID NO:18).

In some embodiments, a 3E10 antibody or variant thereof, or antigen-binding fragment thereof described herein refers to CDR sequences corresponding to the parent 3E10 antibody, shown in FIG. 1, optionally including a D3IN amino acid substitution in the VH CDR1. Accordingly, in some embodiments, a 3E10 antibody or variant thereof, or antigen-binding fragment thereof includes a light chain variable region (VL) complementarity determining region (CDR) 1 comprising the amino acid sequence of 3E10-VL-CDR1 (SEQ ID NO:9), a VL CDR2 comprising the amino acid sequence of 3E10-VL-CDR2 (SEQ ID NO:10), a VL CDR3 comprising the amino acid sequence of 3E10-VL-CDR3 (SEQ ID NO:11), a heavy chain variable region (VH) CDR1 comprising the amino acid sequence of 3E10-VH-CDR1a (SEQ ID NO:16), a VH CDR2 comprising the amino acid sequence of 3E10-VH-CDR2 (SEQ ID NO:4), and a VH CDR3 comprising the amino acid sequence of 3E10-VH-CDR3 (SEQ ID NO:5).

In some embodiments, a 3E10 antibody or variant thereof, or antigen-binding fragment thereof described herein includes CDR sequences corresponding to the parent 3E10 antibody, shown in FIG. 1, with a known amino acid substitution in one or more CDR. For example, FIG. 2B shows the amino acid sequence of several known VH CDR2, VL CDR1, and VL CDR2 amino acid sequences. Accordingly, in some embodiments, a 3E10 antibody or variant thereof, or antigen-binding fragment thereof described herein includes one or more amino acid substitution, relative to the CDR sequences of the parent 3E10 (shown in FIG. 1) or 3E10-D3IN variant (shown in FIG. 2), selected from a G to S substitution at position 5 of VH CDR2, a T to S substitution at position 14 of VH CDR2, an S to T substitution at position 5 of VL CDR1, an M to L substitution at position 14 of VL CDR1, an H to A substitution at position 15 of VL CDR1, and an E to Q substitution at position 6 of VL CDR2.

Accordingly, in some embodiments, a 3E10 antibody or variant thereof, or antigen-binding fragment thereof includes VH CDR2 comprising the amino acid sequence of 3E10-VH-CDR2.1 (SEQ ID NO:26) or 3E10-VH-CDR2.2 (SEQ ID NO:27). In some embodiments, the 3E10 antibody or variant thereof, or antigen-binding fragment thereof further includes VL CDRs 1-3, and VH CDRs 1 and 3 according to the parent 3E10 antibody (as shown in FIG. 1). In some embodiments, the 3E10 antibody or variant thereof, or antigen-binding fragment thereof further includes VL CDRs 1-3, and VH CDRs 1 and 3 according to the 3E10-D3IN variant (as shown in FIG. 2A). In some embodiments, the 3E10 antibody or variant thereof, or antigen-binding fragment thereof further includes VL CDRs 1-3, and VH CDRs 1 and 3 having one or more amino acid substitutions relative to the CDRs of the parent 3E10 antibody (as shown in FIG. 1) or relative to the 3E10-D3IN variant (as shown in FIG. 2A).

Similarly, in some embodiments, a 3E10 antibody or variant thereof, or antigen-binding fragment thereof includes VL CDR1 comprising the amino acid sequence of 3E10-VL-CDR1.1 (SEQ ID NO:28) or 3E10-VL-CDR1.2 (SEQ ID NO:29). In some embodiments, the 3E10 antibody or variant thereof, or antigen-binding fragment thereof further includes VL CDRs 2 and 3, and VH CDRs 1-3 according to the parent 3E10 antibody (as shown in FIG. 1). In some embodiments, the 3E10 antibody or variant thereof, or antigen-binding fragment thereof further includes VL CDRs 2 and 3, and VH CDRs 1-3 according to the 3E10-D3IN variant (as shown in FIG. 2A). In some embodiments, the 3E10 antibody or variant thereof, or antigen-binding fragment thereof further includes VL CDRs 2 and 3, and VH CDRs 1-3 having one or more amino acid substitutions relative to the CDRs of the parent 3E10 antibody (as shown in FIG. 1) or relative to the 3E10-D3IN variant (as shown in FIG. 2A).

Similarly, in some embodiments, a 3E10 antibody or variant thereof, or antigen-binding fragment thereof includes VL CDR2 comprising the amino acid sequence of 3E10-VL-CDR2.1 (SEQ ID NO:30). In some embodiments, the 3E10 antibody or variant thereof, or antigen-binding fragment thereof further includes VL CDRs 1 and 3, and VH CDRs 1-3 according to the parent 3E10 antibody (as shown in FIG. 1). In some embodiments, the 3E10 antibody or variant thereof, or antigen-binding fragment thereof further includes VL CDRs 1 and 3, and VH CDRs 1-3 according to the 3E10-D3IN variant (as shown in FIG. 2A). In some embodiments, the 3E10 antibody or variant thereof, or antigen-binding fragment thereof further includes VL CDRs 1 and 3, and VH CDRs 1-3 having one or more amino acid substitutions relative to the CDRs of the parent 3E10 antibody (as shown in FIG. 1) or relative to the 3E10-D3IN variant (as shown in FIG. 2A).

While some of the amino acid substitutions described above are fairly conservative substitutions—e.g., an S to T substitution at position 5 of VL CDR1—other substitutions are to amino acids that have vastly different properties—e.g., an M to L substitution at position 14 of VL CDR1, an H to A substitution at position 15 of VL CDR1, and an E to Q substitution at position 6 of VL CDR2. This suggests, without being bound by theory, that at least these positions within the 3E10 CDR framework are tolerant to other amino acid substitutions.

Accordingly, in some embodiments, a 3E10 antibody or variant thereof, or antigen-binding fragment thereof includes VH CDR2 comprising the amino acid sequence of 3E10-VH-CDR2.3 (SEQ ID NO:31). In some embodiments, the 3E10 antibody or variant thereof, or antigen-binding fragment thereof further includes VL CDRs 1-3, and VH CDRs 1 and 3 according to the parent 3E10 antibody (as shown in FIG. 1). In some embodiments, the 3E10 antibody or variant thereof, or antigen-binding fragment thereof further includes VL CDRs 1-3, and VH CDRs 1 and 3 according to the 3E10-D3IN variant (as shown in FIG. 2A). In some embodiments, the 3E10 antibody or variant thereof, or antigen-binding fragment thereof further includes VL CDRs 1-3, and VH CDRs 1 and 3 having one or more amino acid substitutions relative to the CDRs of the parent 3E10 antibody (as shown in FIG. 1) or relative to the 3E10-D3IN variant (as shown in FIG. 2A), e.g., as described herein.

Similarly, in some embodiments, a 3E10 antibody or variant thereof, or antigen-binding fragment thereof includes VL CDR1 comprising the amino acid sequence of 3E10-VL-CDR1.3 (SEQ ID NO:32). In some embodiments, the 3E10 antibody or variant thereof, or antigen-binding fragment thereof further includes VL CDRs 2 and 3, and VH CDRs 1-3 according to the parent 3E10 antibody (as shown in FIG. 1). In some embodiments, the 3E10 antibody or variant thereof, or antigen-binding fragment thereof further includes VL CDRs 2 and 3, and VH CDRs 1-3 according to the 3E10-D3IN variant (as shown in FIG. 2A). In some embodiments, the 3E10 antibody or variant thereof, or antigen-binding fragment thereof further includes VL CDRs 2 and 3, and VH CDRs 1-3 having one or more amino acid substitutions relative to the CDRs of the parent 3E10 antibody (as shown in FIG. 1) or relative to the 3E10-D3IN variant (as shown in FIG. 2A), e.g., as described herein.

Similarly, in some embodiments, a 3E10 antibody or variant thereof, or antigen-binding fragment thereof, includes VL CDR2 comprising the amino acid sequence of 3E10-VL-CDR2.2 (SEQ ID NO:33). In some embodiments, the 3E10 antibody or variant thereof, or antigen-binding fragment thereof further includes VL CDRs 1 and 3, and VH CDRs 1-3 according to the parent 3E10 antibody (as shown in FIG. 1). In some embodiments, the 3E10 antibody or variant thereof, or antigen-binding fragment thereof further includes VL CDRs 1 and 3, and VH CDRs 1-3 according to the 3E10-D3IN variant (as shown in FIG. 2A). In some embodiments, the 3E10 antibody or variant thereof, or antigen-binding fragment thereof further includes VL CDRs 1 and 3, and VH CDRs 1-3 having one or more amino acid substitutions relative to the CDRs of the parent 3E10 antibody (as shown in FIG. 1) or relative to the 3E10-D3IN variant (as shown in FIG. 2A), e.g., as described herein.

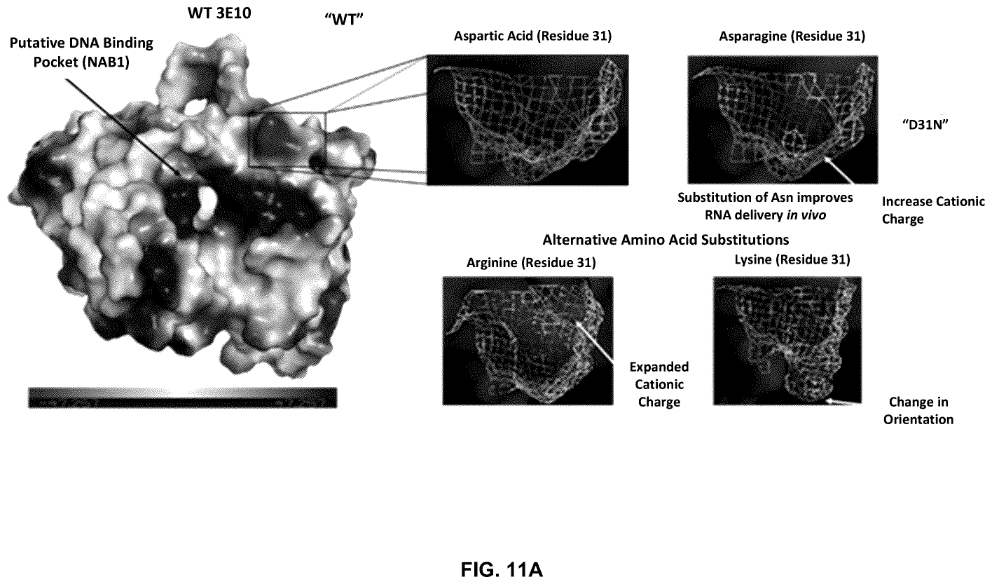

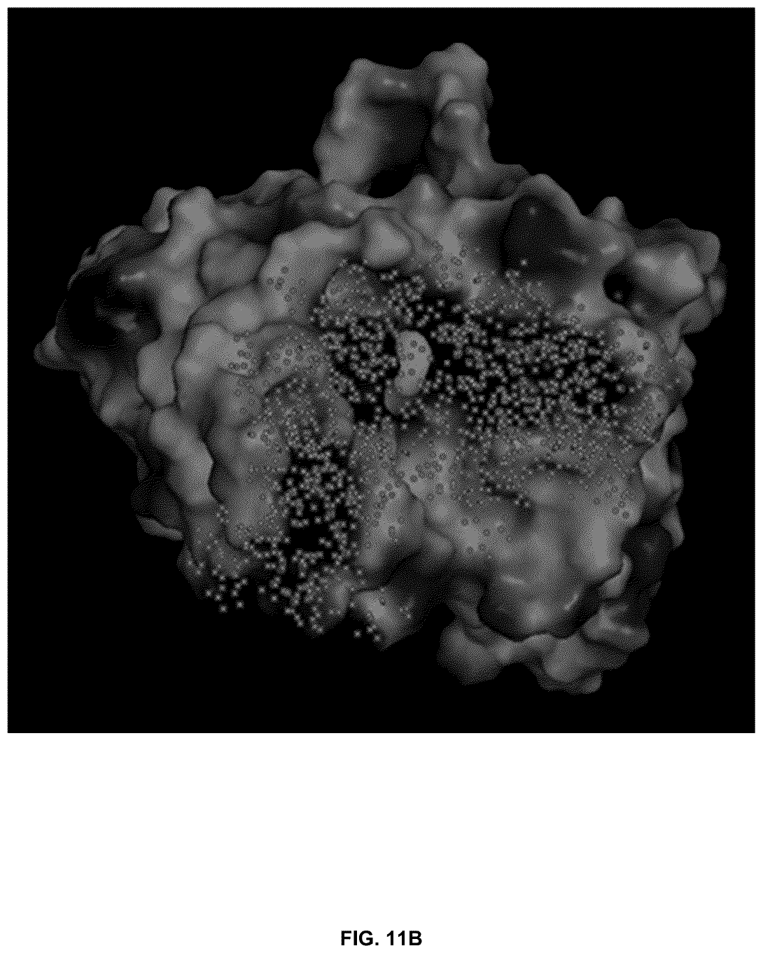

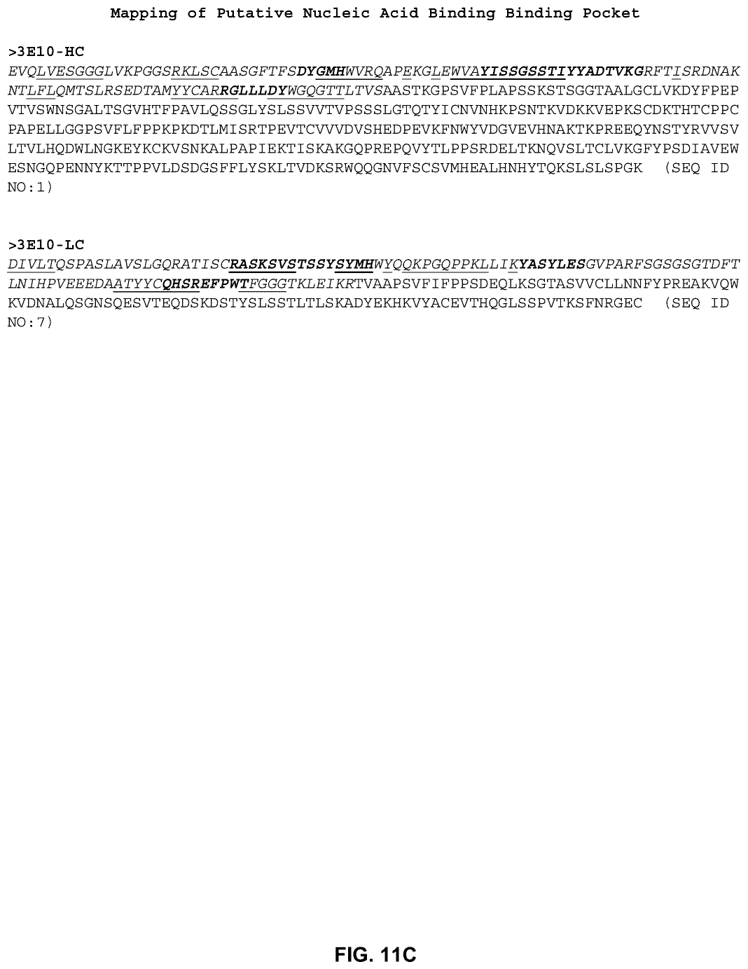

Further, because 3E10 antibodies or variants thereof, or antigen-binding fragments thereof, bind to nucleic acid in a partially sequence-independent manner, and without being bound by theory, it was contemplated that the interaction may be mediated by electrostatic interactions with the nucleotide backbone. To investigate this theory, electrostatic surface potential renderings of a molecular model of a 3E10-scFv construct-the amino acid sequence of which is illustrated in FIG. 11C-were generated, as shown in FIGS. 11A and 11B. These models revealed a putative Nucleic Acid Binding pocket (NAB1) corresponding to a large basic region on the surface of the molecule, as illustrated in FIG. 11A. The position of the non-hydrogen atoms of the amino acids contributing to the putative Nucleic Acid Binding pocket in the model are superposed in FIG. 11B, and the amino acid residues are mapped onto the sequence of the construct in FIG. 11C.

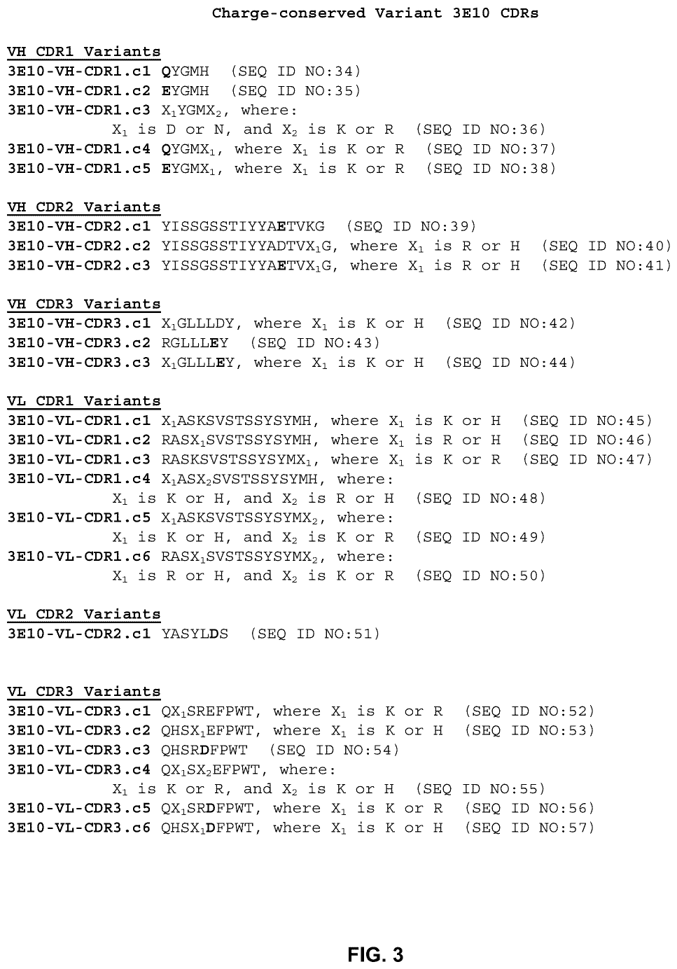

Thus, it is contemplated that amino acid substitutions within the CDRs of a 3E10 antibody or variant thereof, or antigen-binding fragment thereof, as described herein, that maintain the electrostatic character of this putative Nucleic Acid Binding pocket will also retain the nucleic acid binding properties of the construct. Accordingly, in some embodiments, a 3E10 antibody or variant thereof, or antigen-binding fragment thereof, includes one or more amino acid substitution of a first basic amino acid to a second basic amino acid (e.g., K, R, or H). Similarly, in some embodiments, a 3E10 antibody or variant thereof, or antigen-binding fragment thereof, includes one or more amino acid substitution of a first acidic amino acid to a second acidic amino acid (e.g., D or E). Examples of such charge-conserved variant 3E10 CDRs are shown in FIG. 3.

Accordingly, in some embodiments, a 3E10 antibody or variant thereof, or antigen-binding fragment thereof includes VH CDR1 comprising the amino acid sequence of 3E10-VH-CDR1.c1 (SEQ ID NO:34), 3E10-VH-CDR1.c2 (SEQ ID NO:35), 3E10-VH-CDR1.c3 (SEQ ID NO:36), 3E10-VH-CDR1.c4 (SEQ ID NO:37), or 3E10-VH-CDR1.c5 (SEQ ID NO:38). In some embodiments, the 3E10 antibody or variant thereof, or antigen-binding fragment thereof further includes VL CDRs 1-3, and VH CDRs 2 and 3 according to the parent 3E10 antibody (as shown in FIG. 1). In some embodiments, the 3E10 antibody or variant thereof, or antigen-binding fragment thereof further includes VL CDRs 1-3, and VH CDRs 1 and 3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VH CDR2 comprising the amino acid sequence of 3E10-VH-CDR2.c1 (SEQ ID NO:39), 3E10-VH-CDR2.c2 (SEQ ID NO:40), or 3E10-VH-CDR2.c3 (SEQ ID NO:41). In some embodiments, the 3E10 antibody or variant thereof, or antigen-binding fragment thereof further includes VL CDRs 1-3, and VH CDRs 1 and 3 according to the parent 3E10 antibody (as shown in FIG. 1). In some embodiments, the 3E10 antibody or variant thereof, or antigen-binding fragment thereof further includes VL CDRs 1-3, and VH CDRs 1 and 3 according to the 3E10-D3IN variant (as shown in FIG. 2A). In some embodiments, the 3E10 antibody or variant thereof, or antigen-binding fragment thereof further includes VL CDRs 1-3, and VH CDRs 1 and 3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VH CDR3 comprising the amino acid sequence of 3E10-VH-CDR3.c1 (SEQ ID NO:42), 3E10-VH-CDR3.c2 (SEQ ID NO:43), or 3E10-VH-CDR3.c3 (SEQ ID NO:44). In some embodiments, the 3E10 antibody or variant thereof, or antigen-binding fragment thereof further includes VL CDRs 1-3, and VH CDRs 1 and 2 according to the parent 3E10 antibody (as shown in FIG. 1). In some embodiments, the 3E10 antibody or variant thereof, or antigen-binding fragment thereof further includes VL CDRs 1-3, and VH CDRs 1 and 2 according to the 3E10-D3IN variant (as shown in FIG. 2A). In some embodiments, the 3E10 antibody or variant thereof, or antigen-binding fragment thereof further includes VL CDRs 1-3, and VH CDRs 1 and 2 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR1 comprising the amino acid sequence of 3E10-VL-CDR1.c1 (SEQ ID NO:45), 3E10-VL-CDR1.c2 (SEQ ID NO:46), 3E10-VL-CDR1.c3 (SEQ ID NO:47), 3E10-VL-CDR1.c4 (SEQ ID NO:48), 3E10-VL-CDR1.c5 (SEQ ID NO:49), or 3E10-VL-CDR1.c6 (SEQ ID NO:50). In some embodiments, the 3E10 antibody or variant thereof, or antigen-binding fragment thereof further includes VL CDRs 2 and 3, and VH CDRs 1-3 according to the parent 3E10 antibody (as shown in FIG. 1). In some embodiments, the 3E10 antibody or variant thereof, or antigen-binding fragment thereof further includes VL CDRs 2 and 3, and VH CDRs 1-3 according to the 3E10-D3IN variant (as shown in FIG. 2A). In some embodiments, the 3E10 antibody or variant thereof, or antigen-binding fragment thereof further includes VL CDRs 2 and 3, and VH CDRs 1-3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR2 comprising the amino acid sequence of 3E10-VL-CDR2.c1 (SEQ ID NO:51). In some embodiments, the 3E10 antibody or variant thereof, or antigen-binding fragment thereof further includes VL CDRs 1 and 3, and VH CDRs 1-3 according to the parent 3E10 antibody (as shown in FIG. 1). In some embodiments, the 3E10 antibody or variant thereof, or antigen-binding fragment thereof further includes VL CDRs 1 and 3, and VH CDRs 1-3 according to the 3E10-D3IN variant (as shown in FIG. 2A). In some embodiments, the 3E10 antibody or variant thereof, or antigen-binding fragment thereof further includes VL CDRs 1 and 3, and VH CDRs 1-3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR3 comprising the amino acid sequence of 3E10-VL-CDR3.c1 (SEQ ID NO:52), 3E10-VL-CDR3.c2 (SEQ ID NO:53), 3E10-VL-CDR3.c3 (SEQ ID NO:54), 3E10-VL-CDR3.c4 (SEQ ID NO:55), 3E10-VL-CDR3.c5 (SEQ ID NO:56), or 3E10-VL-CDR3.c6 (SEQ ID NO:57). In some embodiments, the 3E10 antibody or variant thereof, or antigen-binding fragment thereof further includes VL CDRs 1 and 2, and VH CDRs 1-3 according to the parent 3E10 antibody (as shown in FIG. 1). In some embodiments, the 3E10 antibody or variant thereof, or antigen-binding fragment thereof further includes VL CDRs 1 and 2, and VH CDRs 1-3 according to the 3E10-D3IN variant (as shown in FIG. 2A). In some embodiments, the 3E10 antibody or variant thereof, or antigen-binding fragment thereof further includes VL CDRs 1 and 2, and VH CDRs 1-3 having one or more amino acid substitutions relative to the CDRs of the parent 3E10 antibody (as shown in FIG. 1), e.g., as described herein.

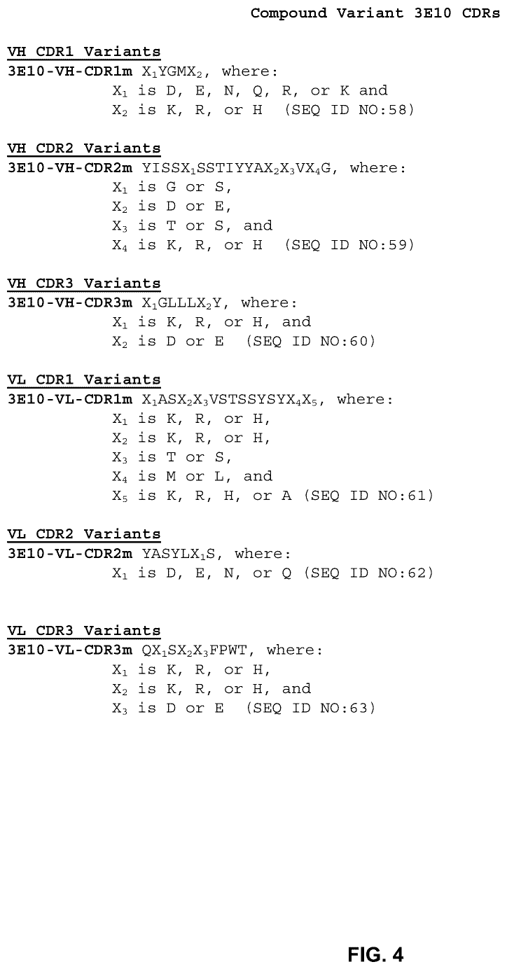

It is also contemplated that a 3E10 antibody or variant thereof, or antigen-binding fragment thereof, as described herein, includes any combination of the 3E10 CDR amino acid substitutions described above. Examples of 3E10 variant CDR sequences that incorporate one or more of the amino acid substitutions described herein are shown in FIG. 4.

Accordingly, in some embodiments, a 3E10 antibody or variant thereof, or antigen-binding fragment thereof includes VH CDR1 comprising the amino acid sequence of 3E10-VH-CDR 1m (SEQ ID NO:58). In some embodiments, the 3E10 antibody or variant thereof, or antigen-binding fragment thereof further includes VL CDRs 1-3, and VH CDRs 2 and 3 according to the parent 3E10 antibody (as shown in FIG. 1). In some embodiments, the 3E10 antibody or variant thereof, or antigen-binding fragment thereof further includes VL CDRs 1-3, and VH CDRs 1 and 3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VH CDR2 comprising the amino acid sequence of 3E10-VH-CDR2m (SEQ ID NO:59). In some embodiments, the 3E10 antibody or variant thereof, or antigen-binding fragment thereof further includes VL CDRs 1-3, and VH CDRs 1 and 3 according to the parent 3E10 antibody (as shown in FIG. 1). In some embodiments, the 3E10 antibody or variant thereof, or antigen-binding fragment thereof further includes VL CDRs 1-3, and VH CDRs 1 and 3 according to the 3E10-D3IN variant (as shown in FIG. 2A). In some embodiments, the 3E10 antibody or variant thereof, or antigen-binding fragment thereof further includes VL CDRs 1-3, and VH CDRs 1 and 3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VH CDR3 comprising the amino acid sequence of 3E10-VH-CDR3m (SEQ ID NO:60). In some embodiments, the 3E10 antibody or variant thereof, or antigen-binding fragment thereof further includes VL CDRs 1-3, and VH CDRs 1 and 2 according to the parent 3E10 antibody (as shown in FIG. 1). In some embodiments, the 3E10 antibody or variant thereof, or antigen-binding fragment thereof further includes VL CDRs 1-3, and VH CDRs 1 and 2 according to the 3E10-D3IN variant (as shown in FIG. 2A). In some embodiments, the 3E10 antibody or variant thereof, or antigen-binding fragment thereof further includes VL CDRs 1-3, and VH CDRs 1 and 2 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR1 comprising the amino acid sequence of 3E10-VL-CDR1m (SEQ ID NO:61). In some embodiments, the 3E10 antibody or variant thereof, or antigen-binding fragment thereof further includes VL CDRs 2 and 3, and VH CDRs 1-3 according to the parent 3E10 antibody (as shown in FIG. 1). In some embodiments, the 3E10 antibody or variant thereof, or antigen-binding fragment thereof further includes VL CDRs 2 and 3, and VH CDRs 1-3 according to the 3E10-D3IN variant (as shown in FIG. 2A). In some embodiments, the 3E10 antibody or variant thereof, or antigen-binding fragment thereof further includes VL CDRs 2 and 3, and VH CDRs 1-3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR2 comprising the amino acid sequence of 3E10-VL-CDR2m (SEQ ID NO:62). In some embodiments, the 3E10 antibody or variant thereof, or antigen-binding fragment thereof further includes VL CDRs 1 and 3, and VH CDRs 1-3 according to the parent 3E10 antibody (as shown in FIG. 1). In some embodiments, the 3E10 antibody or variant thereof, or antigen-binding fragment thereof further includes VL CDRs 1 and 3, and VH CDRs 1-3 according to the 3E10-D3IN variant (as shown in FIG. 2A). In some embodiments, the 3E10 antibody or variant thereof, or antigen-binding fragment thereof further includes VL CDRs 1 and 3, and VH CDRs 1-3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR3 comprising the amino acid sequence of 3E10-VL-CDR3m (SEQ ID NO:63). In some embodiments, the 3E10 antibody or variant thereof, or antigen-binding fragment thereof further includes VL CDRs 1 and 2, and VH CDRs 1-3 according to the parent 3E10 antibody (as shown in FIG. 1). In some embodiments, the 3E10 antibody or variant thereof, or antigen-binding fragment thereof further includes VL CDRs 1 and 2, and VH CDRs 1-3 according to the 3E10-D3IN variant (as shown in FIG. 2A). In some embodiments, the 3E10 antibody or variant thereof, or antigen-binding fragment thereof further includes VL CDRs 1 and 2, and VH CDRs 1-3 having one or more amino acid substitutions relative to the CDRs of the parent 3E10 antibody (as shown in FIG. 1), e.g., as described herein.

In some embodiments, a 3E10 antibody or variant thereof, or antigen-binding fragment thereof described herein includes a light chain variable region (VL) complementarity determining region (CDR) 1 comprising the amino acid sequence of 3E10-VL-CDR1m (SEQ ID NO:61), a VL CDR2 comprising the amino acid sequence of 3E10-VL-CDR2m (SEQ ID NO:62), a VL CDR3 comprising the amino acid sequence of 3E10-VL-CDR3m (SEQ ID NO:63), a heavy chain variable region (VH) CDR1 comprising the amino acid sequence of 3E10-VH-CDR1m (SEQ ID NO:58), a VH CDR2 comprising the amino acid sequence of 3E10-VH-CDR2m (SEQ ID NO:59), and a VH CDR3 comprising the amino acid sequence of 3E10-VH-CDR3m (SEQ ID NO:60).

In some embodiments, a 3E10 antibody or variant thereof, or antigen-binding fragment thereof described herein refers to CDR sequences having no more than one amino acid substitution relative to the parent 3E10 antibody, shown in FIG. 1, optionally including a D3IN amino acid substitution in the VH CDR1. Accordingly, in some embodiments, a 3E10 antibody or variant thereof, or antigen-binding fragment thereof includes a light chain variable region (VL) complementarity determining region (CDR) 1 comprising an amino acid sequence having no more than one amino acid substitution relative to 3E10-VL-CDR1 (SEQ ID NO:9), a VL CDR2 comprising an amino acid sequence having no more than one amino acid substitution relative to 3E10-VL-CDR2 (SEQ ID NO:10), a VL CDR3 comprising an amino acid sequence having no more than one amino acid substitution relative to 3E10-VL-CDR3 (SEQ ID NO:11), a heavy chain variable region (VH) CDR1 comprising an amino acid sequence having no more than one amino acid substitution relative to 3E10-VH-CDR1a (SEQ ID NO:16), a VH CDR2 comprising an amino acid sequence having no more than one amino acid substitution relative to 3E10-VH-CDR2 (SEQ ID NO:4), and a VH CDR3 comprising an amino acid sequence having no more than one amino acid substitution relative to 3E10-VH-CDR3 (SEQ ID NO:5).

In some embodiments, a 3E10 antibody or variant thereof, or antigen-binding fragment thereof described herein refers to CDR sequences having no more than two amino acid substitution relative to the parent 3E10 antibody, shown in FIG. 1, optionally including a D31N amino acid substitution in the VH CDR1. Accordingly, in some embodiments, a 3E10 antibody or variant thereof, or antigen-binding fragment thereof includes a light chain variable region (VL) complementarity determining region (CDR) 1 comprising an amino acid sequence having no more than two amino acid substitutions relative to 3E10-VL-CDR1 (SEQ ID NO:9), a VL CDR2 comprising an amino acid sequence having no more than two amino acid substitutions relative to 3E10-VL-CDR2 (SEQ ID NO:10), a VL CDR3 comprising an amino acid sequence having no more than two amino acid substitutions relative to 3E10-VL-CDR3 (SEQ ID NO:11), a heavy chain variable region (VH) CDR1 comprising an amino acid sequence having no more than two amino acid substitutions relative to 3E10-VH-CDR1a (SEQ ID NO: 16), a VH CDR2 comprising an amino acid sequence having no more than two amino acid substitutions relative to 3E10-VH-CDR2 (SEQ ID NO:4), and a VH CDR3 comprising an amino acid sequence having no more than two amino acid substitutions relative to 3E10-VH-CDR3 (SEQ ID NO:5).

Other variants of a 3E10 antibody or variant thereof, or antigen-binding fragment thereof are also known in the art, as disclosed for example, in Zack, et al., J.

Immunol., 157(5):2082-8 (1996). For example, amino acid position 31 of the heavy chain variable region of 3E10 has been determined to be influential in the ability of the antibody and fragments thereof to penetrate nuclei and bind to DNA (bolded in SEQ ID NOs: 1, 2, and 13). A D31N mutation (bolded in SEQ ID NOs: 2 and 13) in CDR1 penetrates nuclei and binds DNA with much greater efficiency than the original antibody (Zack, et al., Immunology and Cell Biology, 72:513-520 (1994), Weisbart, et al., J. Autoimmun., 11, 539-546 (1998); Weisbart, Int. J. Oncol., 25, 1867-1873 (2004)). In some embodiments, the antibody has the D31N substitution.

Although generally referred to herein as “3E10” or “3E10 antibodies,” it will be appreciated that fragments and binding proteins, including antigen-binding fragments, variants, and fusion proteins such as scFv, di-scFv, tr-scFv, and other single chain variable fragments, and other cell-penetrating, nucleic acid transporting molecules disclosed herein are encompassed by the phrase are also expressly provided for use in compositions and methods disclosed herein. Thus, the antibodies and other binding proteins are also referred to herein as cell-penetrating.

In preferred embodiments, the 3E10 antibody is transported into the cytoplasm and/or nucleus of the cells without the aid of a carrier or conjugate. For example, the monoclonal antibody 3E10 and active fragments thereof that are transported in vivo to the nucleus of mammalian cells without cytotoxic effect are disclosed in U.S. Pat. Nos. 4,812,397 and 7,189,396 to Richard Weisbart.

Antibodies useful in the compositions and methods described herein include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. Therefore, the antibodies typically contain at least the CDRs necessary to maintain DNA binding and/or interfere with DNA repair.

The 3E10 antibody is typically a monoclonal 3E10, or a variant, derivative, fragment, fusion, or humanized form thereof that binds the same or different epitope(s) as 3E10.

A deposit according to the terms of the Budapest Treaty of a hybridoma cell line producing monoclonal antibody 3E10 was received on Sep. 6, 2000, and accepted by, American Type Culture Collection (ATCC), 10801 University Blvd., Manassas, VA 20110-2209, USA, and given Patent Deposit Number PTA-2439.

Thus, the antibody may have the same or different epitope specificity as monoclonal antibody 3E10 produced by ATCC No. PTA 2439 hybridoma. The antibody can have the paratope of monoclonal antibody 3E10. The antibody can be a single chain variable fragment of 3E10, or a variant, e.g., a conservative variant thereof. For example, the antibody can be a single chain variable fragment of 3E10 (3E10 Fv), or a variant thereof.

Additionally, or alternatively, the heavy chain complementarity determining regions (CDRs) can be defined according to the IMGT system. The complementarity determining regions (CDRs) as identified by the IMGT system include

CDR H1.3 (original sequence):
(SEQ ID NO: 99)
GFTFSDYG;
CDR H1.4
(with D31N mutation):
(SEQ ID NO: 100)
GFTFSNYG;
CDR H2.2:
(SEQ ID NO: 101)
ISSGSSTI
and
variant
(SEQ ID NO: 102)
ISSSSSTI;
CDR H3.2:
(SEQ ID NO: 103)
ARRGLLLDY

Additionally, or alternatively, the light chain complementarity determining regions (CDRs) can be defined according to the IMGT system. The complementarity determining regions (CDRs) as identified by the IMGT system include CDR L1.2 KSVSTSSYSY (SEQ ID NO:104) and variant KTVSTSSYSY (SEQ ID NO:105); CDR L2.2: YAS (SEQ ID NO:106); CDR L3.2: QHSREFPWT (SEQ ID NO:107).

In some embodiments, the antibody is a humanized antibody. Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule.

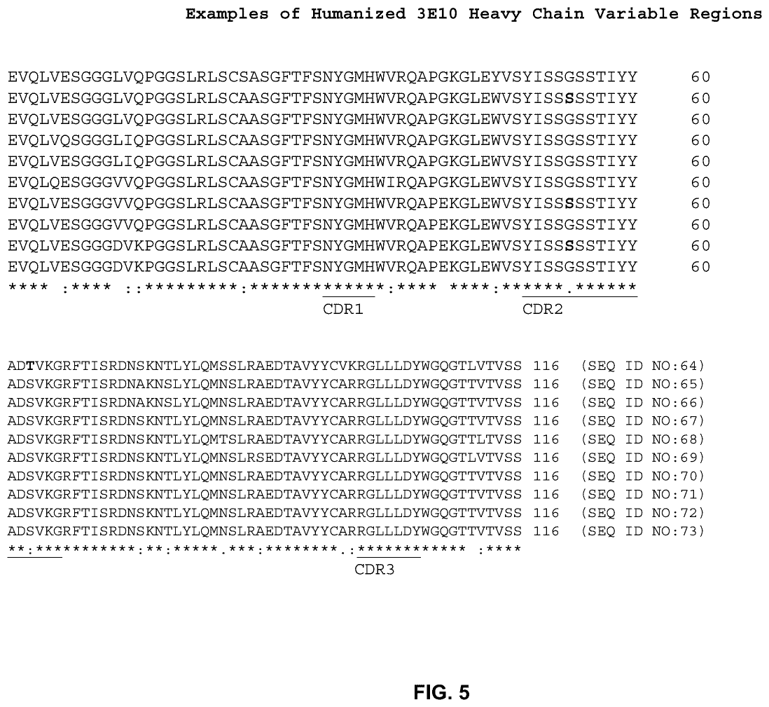

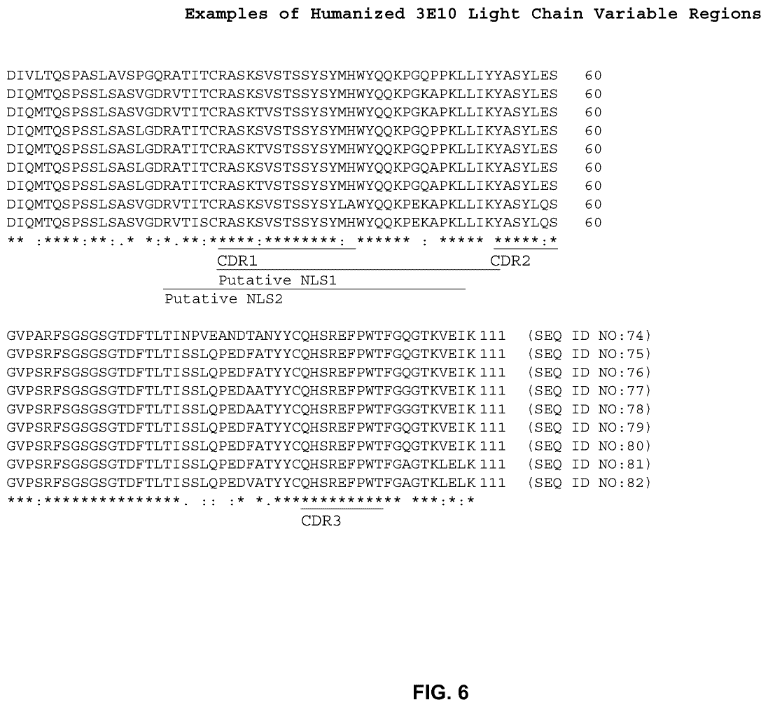

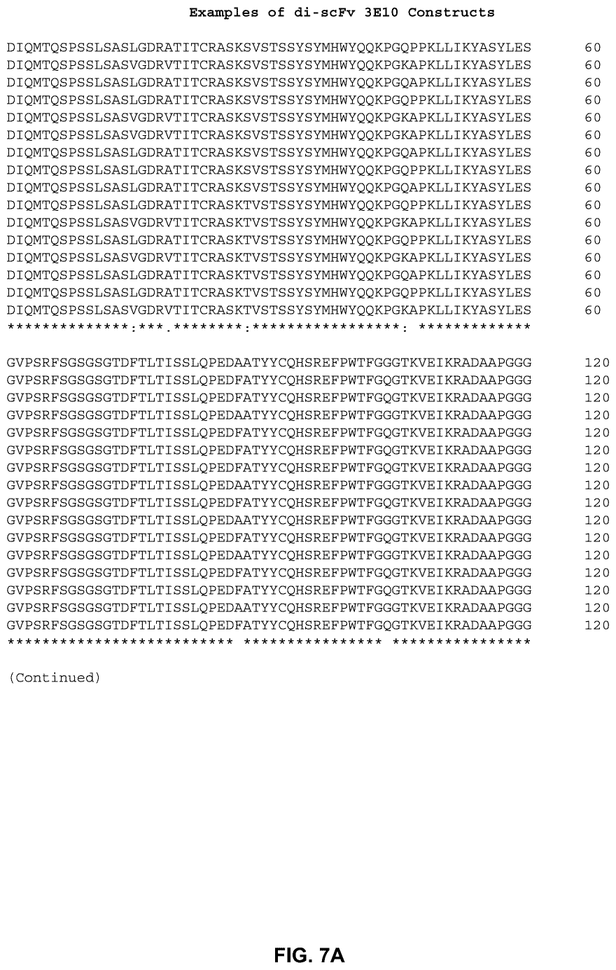

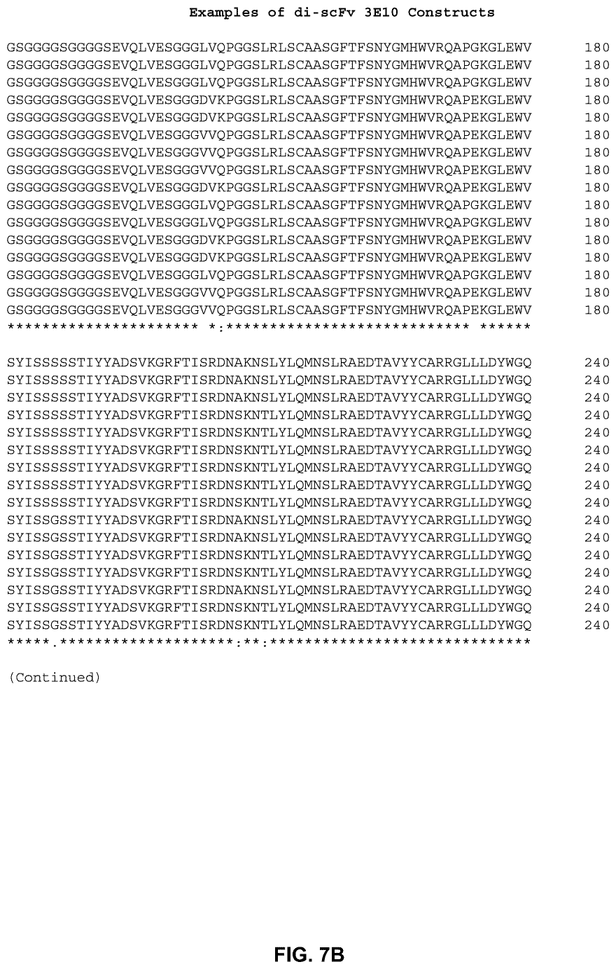

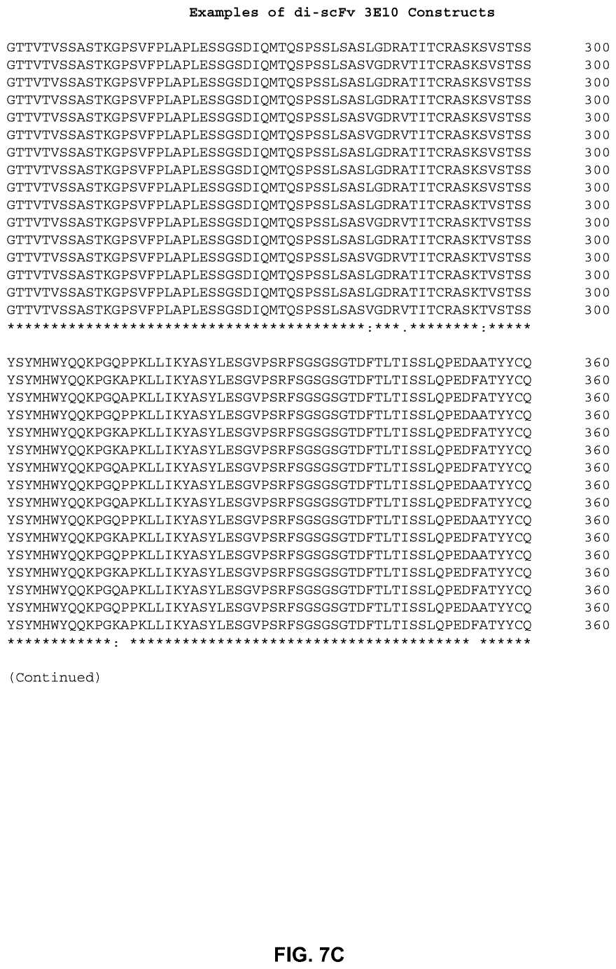

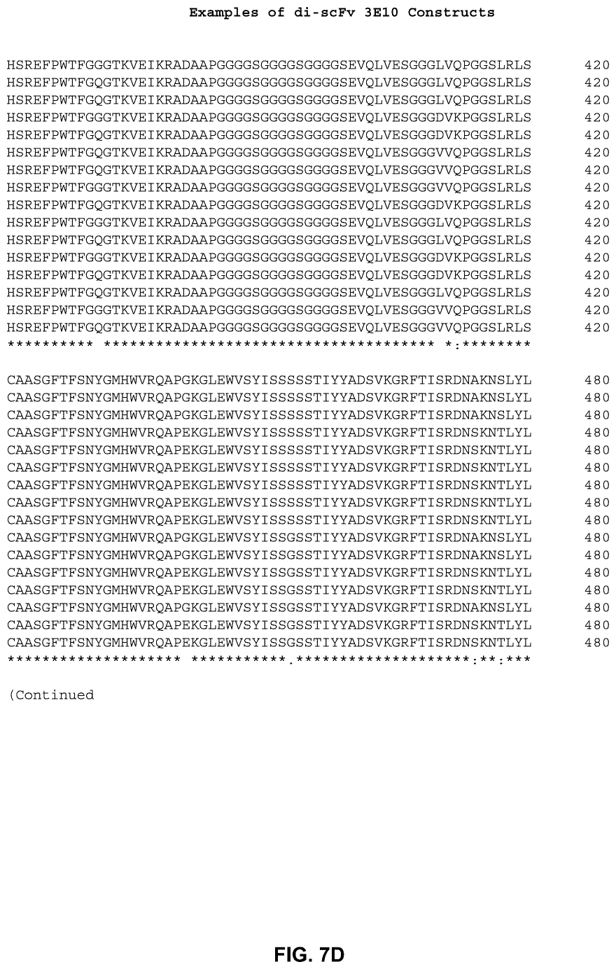

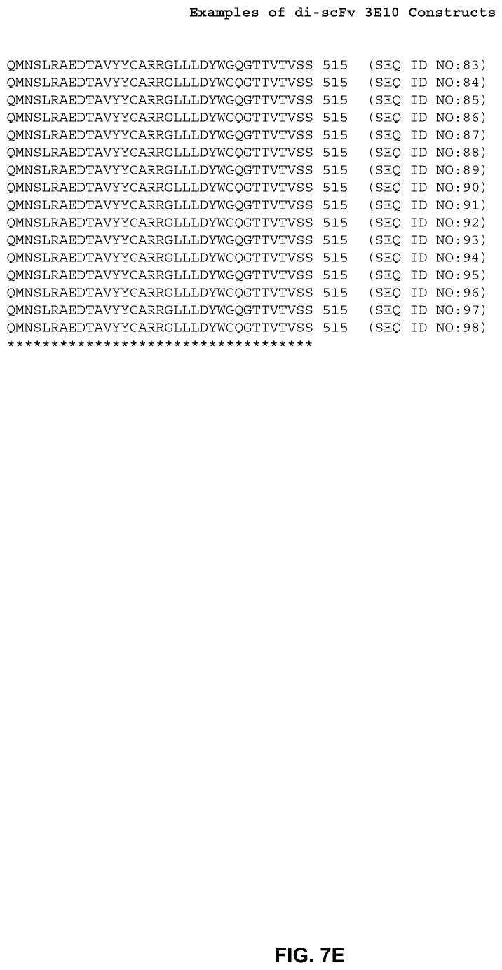

Exemplary 3E10 humanized 3E10 heavy chain variable region (SEQ ID NOs:64-73) and light chain variable region (SEQ ID NOs:74-82) sequences are discussed in WO 2015/106290 (U.S. Pat. No. 10,221,250), WO 2016/033324 (U.S. Pat. No. 10,501,554), WO 2019/018426, and WO/2019/018428 (US 2020/216568), the disclosures of which are incorporated herein by reference in their entireties for all purposes, and provided in FIGS. 5 and 6, respectively. In some embodiments, the 3E10 antibodies described herein include a heavy chain variable region selected from SEQ ID NOs:64-73 and a light chain variable region selected from SEQ ID NOs:74-82. In some embodiments, the 3E10 antibodies described herein include a heavy chain variable region having at least 99% amino acid identity with a heavy chain variable region selected from SEQ ID NOs:64-73 and a light chain variable region having at least 99% amino acid identity with a light chain variable region selected from SEQ ID NOs: 74-82. In some embodiments, the 3E10 antibodies described herein include a heavy chain variable region having at least 98% amino acid identity with a heavy chain variable region selected from SEQ ID NOs:64-73 and a light chain variable region having at least 98% amino acid identity with a light chain variable region selected from SEQ ID NOs: 74-82. In some embodiments, the 3E10 antibodies described herein include a heavy chain variable region having at least 97% amino acid identity with a heavy chain variable region selected from SEQ ID NOs:64-73 and a light chain variable region having at least 97% amino acid identity with a light chain variable region selected from SEQ ID NOs:74-82. In some embodiments, the 3E10 antibodies described herein include a heavy chain variable region having at least 96% amino acid identity with a heavy chain variable region selected from SEQ ID NOs:64-73 and a light chain variable region having at least 96% amino acid identity with a light chain variable region selected from SEQ ID NOs:74-82. In some embodiments, the 3E10 antibodies described herein include a heavy chain variable region having at least 95% amino acid identity with a heavy chain variable region selected from SEQ ID NOs:64-73 and a light chain variable region having at least 95% amino acid identity with a light chain variable region selected from SEQ ID NOs: 74-82. In some embodiments, the 3E10 antibodies described herein include a heavy chain variable region having at least 90%, 91%, 92%, 93%, or 94% amino acid identity with a heavy chain variable region selected from SEQ ID NOs:64-73 and a light chain variable region having at least 90%, 91%, 92%, 93%, or 94% amino acid identity with a light chain variable region selected from SEQ ID NOs:74-82.

The disclosed compositions and methods typically utilize antibodies that maintain the ability to penetrate cells, and optionally nuclei. The mechanisms of cellular internalization by autoantibodies are diverse. Some are taken into cells through electrostatic interactions or FcR-mediated endocytosis, while others utilize mechanisms based on association with cell surface myosin or calreticulin, followed by endocytosis (Ying-Chyi et al., Eur J Immunol 38, 3178-3190 (2008), Yanase et al., J Clin Invest 100, 25-31 (1997)). 3E10 penetrates cells in an Fc-independent mechanism (as evidenced by the ability of 3E10 fragments lacking an Fc to penetrate cells) but involves presence of the nucleoside transporter ENT2 (Weisbart et al., Sci Rep 5:12022. doi: 10.1038/srep12022. (2015), Zack et al., J Immunol 157, 2082-2088 (1996), Hansen et al., J Biol Chem 282, 20790-20793 (2007)). Thus, in some embodiments, the antibodies utilized in the disclosed compositions and methods are ones that penetrates cells in an Fc-independent mechanism but involves presence of the nucleoside transporter ENT2.

Mutations in 3E10 that interfere with its ability to bind DNA may render the antibody incapable of nuclear penetration. Thus, typically the disclosed variants and humanized forms of the antibody maintain the ability to bind nucleic acids, particularly DNA. In addition, 3E10 scFv has previously been shown capable of penetrating into living cells and nucleic in an ENT2-dependent manner, with efficiency of uptake impaired in ENT2-deficient cells (Hansen, et al., J. Biol. Chem. 282, 20790-20793 (2007)). Thus, in some embodiments, the disclosed variants and humanized forms of the antibody maintain the ability penetrate into cell nuclei in an ENT-dependent, preferably ENT2-dependent manner.

### Bispecific Constructs

The anti-DNA antibodies can be modified to improve their therapeutic potential. For example, in some embodiments, the cell-penetrating anti-DNA antibody is conjugated to another antibody specific for a second therapeutic target in the cytoplasm and/or nucleus of a target cell. For example, the cell-penetrating anti-DNA antibody can be a fusion protein containing 3E10 Fv and a single chain variable fragment of a monoclonal antibody that specifically binds the second therapeutic target. In other embodiments, the cell-penetrating anti-DNA antibody is a bispecific antibody having a first heavy chain and a first light chain from 3E10 and a second heavy chain and a second light chain from a monoclonal antibody that specifically binds the second therapeutic target.

Bispecific antibodies and other binding proteins having a first heavy chain and a first light chain from 3E10 and a second heavy chain and a second light chain from a monoclonal antibody that specifically binds a second target are discussed in Weisbart, et al., Mol. Cancer Ther., 11(10):2169-73 (2012), and Weisbart, et al., Int. J. Oncology, 25:1113-8 (2004), and U.S. Patent Application No. 2013/0266570, which are specifically incorporated by reference in their entireties. In some embodiments, the second target is specific for a target cell-type, tissue, organ etc. Thus the second heavy chain and second light chain can serve as a targeting moiety that targets the complex to the target cell-type, tissue, organ. In some embodiments, the second heavy chain and second light chain target, hematopoietic stem cells, CD34+ cells, T cells or any another preferred cell type, e.g., by targeting a receptor or ligand expressed on the preferred cell type. In some embodiments, the second heavy chain and second light chain target the thymus, spleen, or cancer cells.

In some embodiments, particularly those for targeting T cell in vivo, for example, for in vivo production of CAR T cells, immune cell or T cell markers such as CD3, CD7, or CD8 can be targeted. For example, anti-CD8 antibodies and anti-CD3 Fab fragments have both been used to target T cells in vivo (Pfeiffer, et al., EMBO Mol Med., 10(11) (2018). pii: e9158. doi: 10.15252/emmm.201809158., Smith, et al., Nat Nanotechnol., 12(8):813-820 (2017). doi: 10.1038/nnano.2017.57). Thus, in some embodiments, the 3E10 antibody or antigen binding fragment or fusion protein is a bispecific antibody part of which can bind specifically to CD3, CD7, CD8, or another immune cell (e.g., T cell) marker, or a marker for a specific tissue such as the thymus, spleen, or liver.

### Nucleic Acid Binding

The disclosed compositions and methods utilize 3E10 antibodies or variants thereof, or antigen-binding fragments thereof that bind to polynucleotides. Example 4 describes molecular modeling of 3E10 and additional 3E10 variants. Molecular modeling of 3E10 (Pymol) reveals a putative Nucleic Acid Binding pocket (NAB1) (see, e.g., FIGS. 11A and 11B, and illustrated with underlining in FIG. 11C).

In some embodiments, the disclosed antibodies include some or all of the underlined NAB1 sequences. In some embodiments, the antibodies include a variant sequence that has an altered ability of bind nucleic acids. In some embodiments, the mutations (e.g., substitutions, insertions, and/or deletions) in the NAB1 improve binding of the antibody to nucleic acids such as RNA. In some embodiments, the mutations are conservative substitutions. In some embodiments, the mutations increase the cationic charge of the NAB1 pocket.

As discussed and exemplified herein, mutation of aspartic acid at residue 31 of CDR1 to asparagine increased the cationic charge of this residue and enhanced nucleic acid binding and delivery in vivo (3E10-D31N). Additional exemplary variants include mutation of aspartic acid at residue 31 of CDR1 to arginine (3E10-D31R), which modeling indicates expands cationic charge, or lysine (3E10-D31K) which modeling indicates changes charge orientation. Thus, in some embodiments, the 3E10 binding protein includes a D31R or D31K substitution.

All of the sequences disclosed herein having the residue corresponding to 3E10 D31 or N31, are expressly disclosed with a D31R or D31K or N31R or N31K substitution therein.

Molecular modeling of 3E10 (Pymol) revealed a putative Nucleic Acid Binding pocket (NAB1) (FIGS. 11A-11B). Mutation of aspartic acid at residue 31 of CDR1 to asparagine increased the cationic charge of this residue and enhanced nucleic acid binding and delivery in vivo (3E10-D31N). Mutation of aspartic acid at residue 31 of CDR1 to arginine (3E10-D31R), further expanded the cationic charge while mutation to lysine (3E10-D31K) changed charge orientation (FIG. 11A).

NAB1 amino acids predicted from molecular modeling have been underlined in the heavy and light chain sequences in FIG. 11C. FIG. 11B is an illustration showing molecular modeling of 3E10-scFv (Pymol) with NAB1 amino acid residues illustrated with punctate dots.

### Therapeutic Polynucleotides

The present disclosure is based, at least in part, on the discovery that 3E10 antibodies or variants thereof, or antigen-binding fragments thereof bind to some polynucleotide sequences with greater affinity than they bind to other polynucleotide sequences. Without being bound by theory, this result suggests that the effectiveness of 3E10 and 3E10 variant (e.g., 3E10 (D31N))-based polynucleotide delivery systems could be enhanced, at least in part, by the sequence of the polynucleotide being delivered. As such, it was contemplated that the effectiveness of such a delivery system could be improved by appending a polynucleotide having a preferred 3E10 or 3E10 variant binding sequence (referred to herein as a “3E10 or 3E10 variant binding domain”) to an effector polynucleotide, thereby providing a molecular handle that a 3E10 antibody or variant thereof, or antigen-binding fragment thereof can bind to with high affinity. That is, it is expected that a therapeutic polynucleotide having a 3E10 or 3E10 variant binding domain will be bound more tightly by a 3E10 antibody or variant thereof, or antigen-binding fragment thereof than will the effector polynucleotide alone, i.e., lacking the 3E10 or 3E10 variant binding domain.

As used herein, the term “effector domain” refers to a portion of a polynucleotide that is combined with a 3E10 or 3E10 variant binding domain, and is used herein for purposes of clarity and/or brevity rather than to connote a particular therapeutic effect. While, in certain embodiments, part of an effector domain provides a therapeutic effect in certain physiological contexts, it is not strictly necessary.

3E10 or 3E10 Variant Binding Domain

In some embodiments, a 3E10 or 3E10 variant binding domain is a single-stranded polynucleotide that is combined with an effector domain in a polynucleotide for delivery.

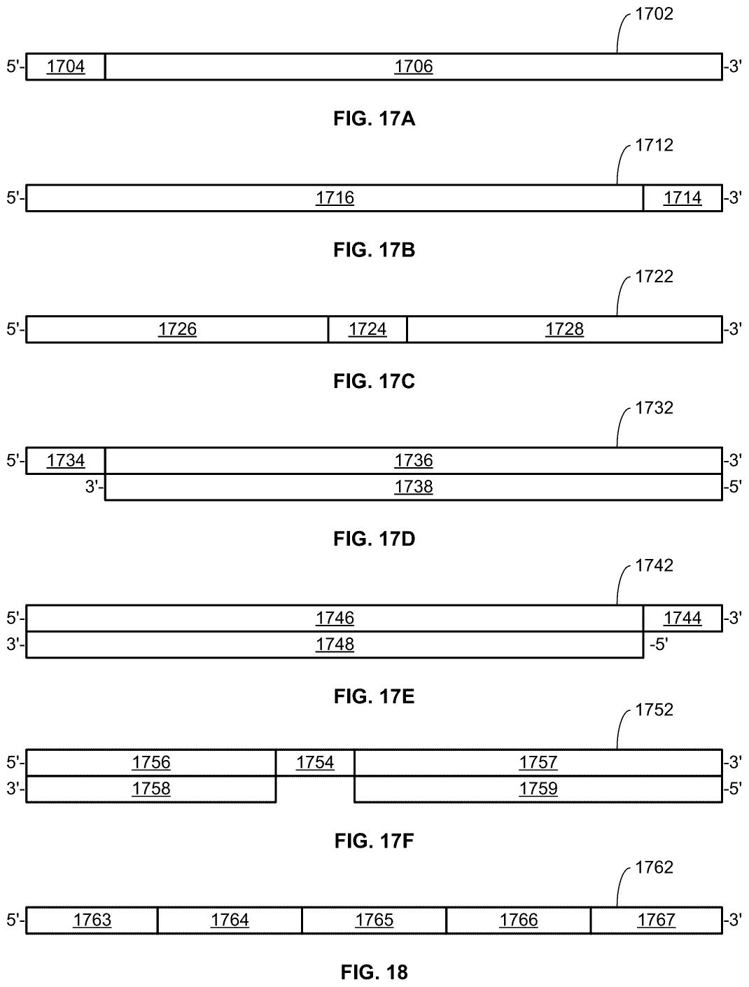

Generally, a 3E10 or 3E10 variant binding domain can be incorporated at any position of a polynucleotide desired to be delivered, including at the 5′ end, the 3′ end, or at an internal position. In some embodiments, a coding region can be codon optimized for 3E10 or 3E10 variant binding. For instance, FIGS. 17A-17F illustrate several example architectures for a therapeutic polynucleotide having a 3E10 or 3E10 variant binding domain, in accordance with various embodiments of the present disclosure. Although exclusively illustrated as linear molecules in FIG. 17, the therapeutic polynucleotides described herein can have other topologies as well, such as circularized forms and/or concatenated forms comprising multiple polynucleotides.

In some embodiments, a single-stranded 3E10 or 3E10 variant binding domain is incorporated into a single-stranded therapeutic polynucleotide, examples of which are illustrated in FIGS. 17A-17C. In some embodiments, a single-stranded 3E10 or 3E10 variant binding domain is incorporated into a double-stranded therapeutic polynucleotide, examples of which are illustrated in FIGS. 17D-17F.

Accordingly, in some embodiments, a 3E10 or 3E10 variant binding domain (e.g., binding domain 1704 in FIG. 17A) is positioned at the 5′ end of a single-stranded therapeutic polynucleotide (e.g., therapeutic polynucleotide 1702 in FIG. 17A) that includes an effector domain (e.g., effector domain 1706 in FIG. 17A).

Similarly, in some embodiments, a 3E10 or 3E10 variant binding domain (e.g., binding domain 1714 in FIG. 17B) is positioned at the 3′ end of a single-stranded therapeutic polynucleotide (e.g., therapeutic polynucleotide 1712 in FIG. 17B) that includes an effector domain (e.g., effector domain 1716 in FIG. 17B).

Likewise, in some embodiments, a 3E10 or 3E10 variant binding domain (e.g., binding domain 1724 in FIG. 17C) is positioned internally of a single-stranded therapeutic polynucleotide (e.g., therapeutic polynucleotide 1722 in FIG. 17C) that includes an effector domain (e.g., effector domains 1726 and 1728, which may be separate effector domains or be two parts to a single, e.g., compound, effector domain, in FIG. 17C).

In some embodiments, a 3E10 or 3E10 variant binding domain (e.g., binding domain 1734 in FIG. 17D) is positioned at the 5′ end of one strand of a double-stranded therapeutic polynucleotide (e.g., therapeutic polynucleotide 1732 in FIG. 17D) that includes an effector domain (e.g., a double-stranded effector domain containing polynucleotide strands 1736 and 1738 in FIG. 17D).

Similarly, in some embodiments, a 3E10 or 3E10 variant binding domain (e.g., binding domain 1744 in FIG. 17E) is positioned at the 3′ end of one strand of a double-stranded therapeutic polynucleotide (e.g., therapeutic polynucleotide 1742 in FIG. 17E) that includes an effector domain (e.g., a double-stranded effector domain containing polynucleotide strands 1746 and 1748 in FIG. 17E).

Likewise, in some embodiments, a 3E10 or 3E10 variant binding domain (e.g., binding domain 1754 in FIG. 17F) is positioned internally of a double-stranded therapeutic polynucleotide (e.g., therapeutic polynucleotide 1752 in FIG. 17F) that includes an effector domain (e.g., double-stranded effector domains containing polynucleotide strands 1756/1757 and 1758/1759, which may be separate effector domains or be two parts to a single, e.g., compound, effector domain, in FIG. 17F).

In some embodiments, a 3E10 or 3E10 variant binding domain is a single-stranded polynucleotide, e.g., a polyribonucleotide or a polydeoxyribonucleotide, at least 10 nucleotides in length. In some embodiments, the 3E10 or 3E10 variant binding domain is at least 25 nucleotides in length. In some embodiments, the 3E10 or 3E10 variant binding domain is at least 50 nucleotides in length. In some embodiments, the 3E10 or 3E10 variant binding domain is at least 100 nucleotides in length. In some embodiments, the 3E10 or 3E10 variant binding domain is at least about 15, at least about 20, at least about 25, at least about 30, at least about 35, at least about 40, at least about 45, at least about 50, at least about 60, at least about 70, at least about 75, at least about 80, at least about 90, at least about 100, at least about 125, at least about 150, at least about 175, at least about 200, at least about 250, at least about 300, or more nucleotides in length.

In some embodiments, a 3E10 or 3E10 variant binding domain is no more than 500 nucleotides in length. In some embodiments, the 3E10 or 3E10 variant binding domain no more than 250 nucleotides in length. In some embodiments, the 3E10 or 3E10 variant binding domain is no more than 100 nucleotides in length. In some embodiments, the 3E10 or 3E10 variant binding domain is no more than about 500, no more than about 450, no more than about 400, no more than about 350, no more than about 300, no more than about 250, no more than about 200, no more than about 150, no more than about 100, no more than about 75, no more than about 50, or fewer nucleotides in length.

In some embodiments, a 3E10 or 3E10 variant binding domain is from about 10 nucleotides to about 500 nucleotides in length. In some embodiments, a 3E10 or 3E10 variant binding domain is from about 25 nucleotides to about 250 nucleotides in length. In some embodiments, a 3E10 or 3E10 variant binding domain is from about 50 nucleotides to about 100 nucleotides in length. In some embodiments, a 3E10 or 3E10 variant binding domain is from about 25 nucleotides to about 100 nucleotides in length. Other suitable ranges of nucleotide length, falling within the example ranges provided above, are also contemplated for use in the methods and compositions described herein.

As reported in Examples 7 and 8 below, the affinity of a 3E10 antibody or variant thereof, or antigen-binding fragment thereof is in part dependent upon at least (i) the type of nucleic acid being bound, e.g., ribonucleotides or deoxyribonucleotides, (ii) the identity of the nucleotide bases, e.g., adenine (A), cytosine (C), guanine (G), thymine (T), uracil (U), inosine (I), etc., and (iii) the identity of the amino acid residue at position 1 of the heavy chain CDR1, also referred to herein as heavy chain (HC) amino acid 31, HC CDR1 amino acid 1, and D31X, where X refers to the identity amino acid, e.g., D3IN refers to variant CDR sequence in which the parental aspartic acid residue has been replaced with asparagine.

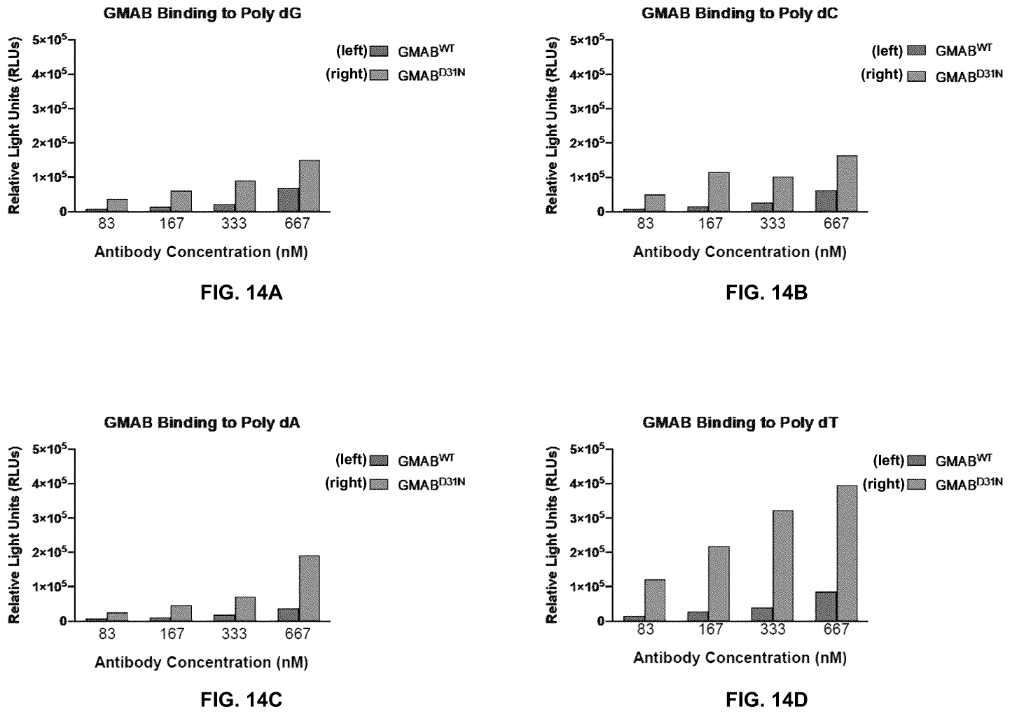

For instance, as shown in FIGS. 14A-14D, 3E10 (D31N) variants have a greater affinity for poly-dT sequences than for poly-dA, poly-dC, and poly-dG sequences. Accordingly, in some embodiments, a 3E10 or 3E10 variant binding domain includes a poly-dT nucleotide sequence. In some embodiments, a 3E10 or 3E10 variant binding domain is a poly-dT polydeoxyribonucleotide. In some embodiments, a 3E10 or 3E10 variant binding domain has a dT content of at least 25%, i.e., a content of from 25% to 100%. In some embodiments, a 3E10 or 3E10 variant binding domain has a dT content of at least 50%, i.e., a content of from 50% to 100%. In some embodiments, a 3E10 or 3E10 variant binding domain has a dT content of at least 75%, i.e., a content of from 75% to 100%. In some embodiments, a 3E10 or 3E10 variant binding domain has a dT content of at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or greater.

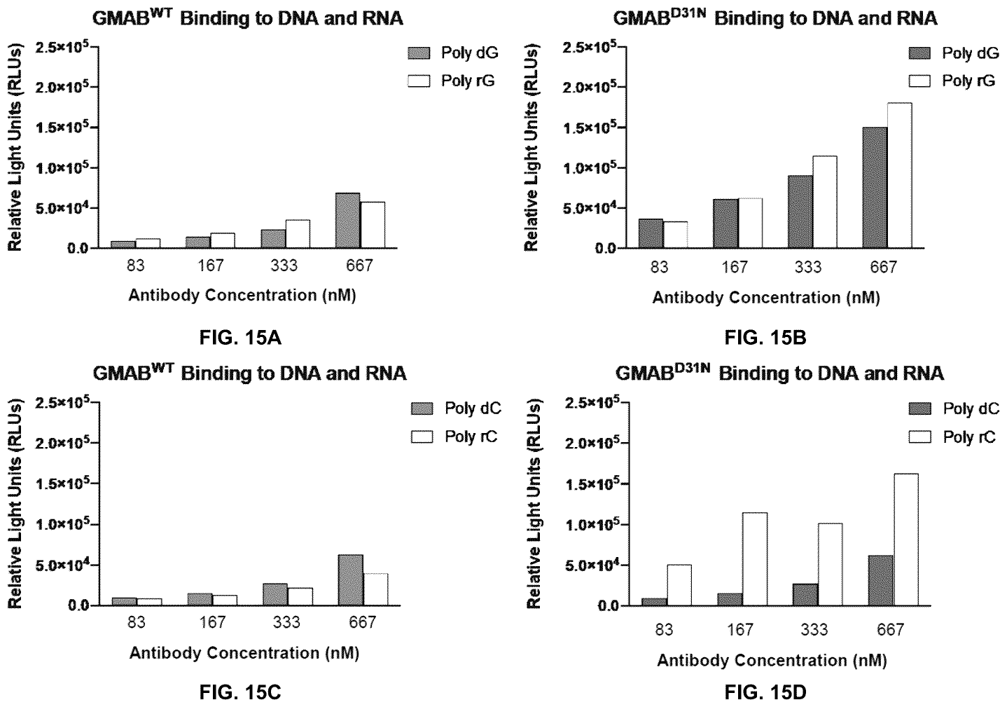

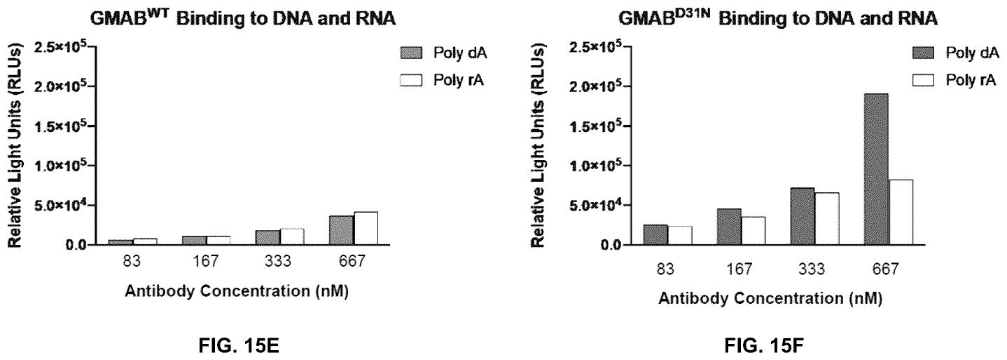

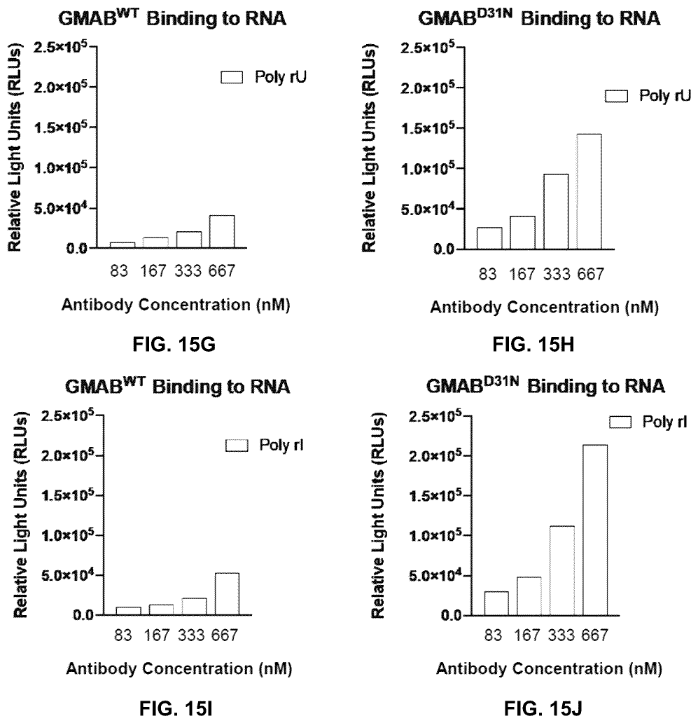

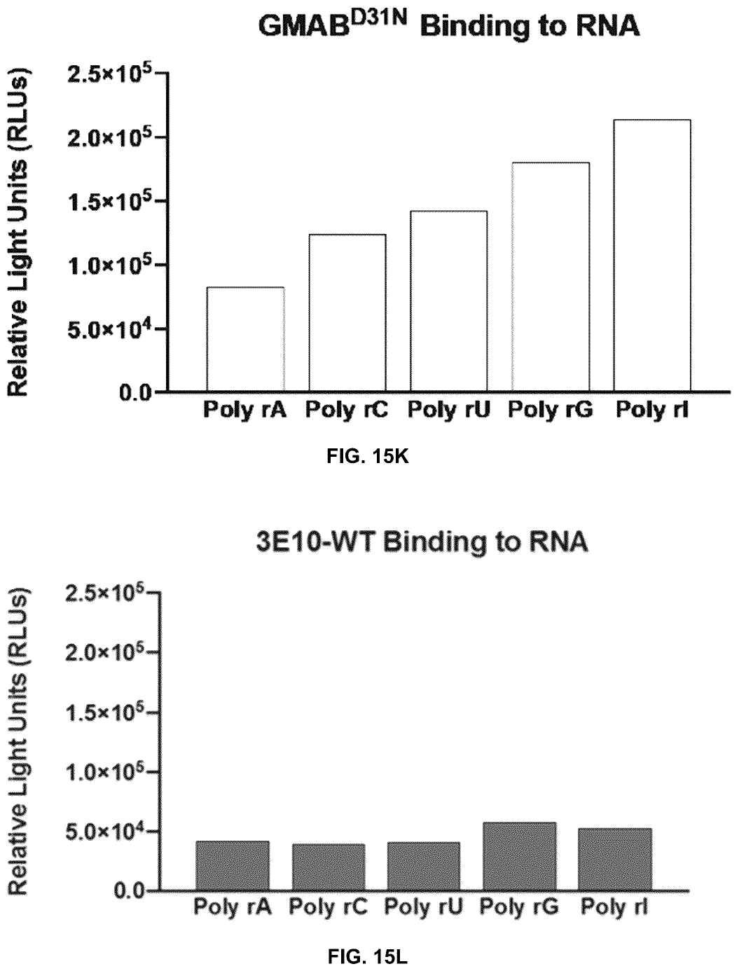

As shown in FIG. 15A-15K, 3E10 (D3IN) variants, appear to have a greater affinity for poly-rI sequences than for poly-rA, poly-rC, poly-rG, and poly-rU sequences. Additionally, 3E10 parental constructs appear to bind poly-rl about as well, if not better, than poly-rA, poly-rC, poly-rG, and poly-rU sequences. Accordingly, in some embodiments, a 3E10 or 3E10 variant binding domain includes a poly-rl nucleotide sequence. In some embodiments, a 3E10 or 3E10 variant binding domain is a poly-rI polydeoxyribonucleotide. In some embodiments, a 3E10 or 3E10 variant binding domain has a rI content of at least 25%, i.e., a content of from 25% to 100%. In some embodiments, a 3E10 or 3E10 variant binding domain has a rI content of at least 50%, i.e., a content of from 50% to 100%. In some embodiments, a 3E10 or 3E10 variant binding domain has a rI content of at least 75%, i.e., a content of from 75% to 100%. In some embodiments, a 3E10 or 3E10 variant binding domain has a rI content of at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or greater.

Similarly, as shown in FIG. 15A-15K, 3E10 (D31N) variants, appear to have a greater affinity for poly-rG sequences than for poly-rA, poly-rC, and poly-rU sequences. Additionally, 3E10 parental constructs appear to bind poly-rG about as well, if not better, than poly-rA, poly-rC, poly-rI, and poly-rU sequences. Accordingly, in some embodiments, a 3E10 or 3E10 variant binding domain includes a poly-rG nucleotide sequence. In some embodiments, a 3E10 or 3E10 variant binding domain is a poly-rG polydeoxyribonucleotide. In some embodiments, a 3E10 or 3E10 variant binding domain has a rG content of at least 25%, i.e., a content of from 25% to 100%. In some embodiments, a 3E10 or 3E10 variant binding domain has a rG content of at least 50%, i.e., a content of from 50% to 100%. In some embodiments, a 3E10 or 3E10 variant binding domain has a rG content of at least 75%, i.e., a content of from 75% to 100%. In some embodiments, a 3E10 or 3E10 variant binding domain has a rG content of at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or greater.

Likewise, in some embodiments, a 3E10 or 3E10 variant binding domain has a combined rl and rG content of at least 50%, i.e., a content of from 50% to 100%. In some embodiments, a 3E10 or 3E10 variant binding domain has a combined rl and rG content of at least 75%, i.e., a content of from 75% to 100%. In some embodiments, a 3E10 or 3E10 variant binding domain has a combined rl and rG content of at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or greater.

Conversely, as shown in FIGS. 15A-15K, 3E10, parental 3E10 and 3E10 (D31N) variants appear to have lower affinity for poly-rA sequences than for poly-rG, poly-rC, poly-rI, and poly-rU sequences. Accordingly, in some embodiments, a 3E10 or 3E10 variant binding domain has a rA content of no more than 25%, i.e., a content of from 0% to 25%. In some embodiments, a 3E10 or 3E10 variant binding domain has a rA content of no more than 20%, i.e., a content of from 0% to 20%. In some embodiments, a 3E10 or 3E10 variant binding domain has a rA content of no more than 10%, i.e., a content of from 0% to 10%. In some embodiments, a 3E10 or 3E10 variant binding domain has a rA content of no more than about 25%, no more than about 20%, no more than about 15%, no more than about 10%, no more than about 5%, no more than about 2%, no more than about 1%, or less.

In some embodiments, the affinity of a 3E10 antibody or variant thereof, or antigen-binding fragment thereof for a 3E10 or 3E10 variant binding domain is at least 25% greater than the average affinity of the 3E10 antibody or variant thereof, or antigen-binding fragment thereof for a second polynucleotide having the first length and a second nucleotide sequence having evenly-balanced A, T/U, C, and G content of 25% each. In some embodiments, the affinity of a 3E10 antibody or variant thereof, or antigen-binding fragment thereof for a 3E10 or 3E10 variant binding domain is at least about 5%, at least about 10%, at least about 15%, at least about 20%, at least about 25%, at least about 30%, at least about 35%, at least about 40%, at least about 45%, at least about 50%, at least about 60%, at least about 70%, at least about 75%, at least about 80%, at least about 90%, at least about 100%, at least about 125%, at least about 150%, at least about 175%, at least about 200%, at least about 250%, at least about 300%, at least about 400%, at least about 500%, or more greater than the average affinity of the 3E10 antibody or variant thereof, or antigen-binding fragment thereof for a second polynucleotide having the first length and a second, random nucleotide sequence

In some embodiments, a 3E10 or 3E10 variant binding domain includes one or more non-canonical nucleotides, e.g., to improve the stability and/or half-life of the therapeutic polynucleotide in vivo. Examples of non-canonical nucleotides suitable for inclusion in the therapeutic polynucleotide described herein are described in U.S. Pat. No. 9,181,319, the content of which is incorporated herein by reference.

In some embodiments, a 3E10 or 3E10 variant binding domain includes one or more non-canonical uridine nucleotide analog. Non-limited examples of non-canonical uridine nucleotide analogs include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, a 3E10 or 3E10 variant binding domain includes one or more non-canonical cytidine nucleotide analog. Non-limited examples of non-canonical cytidine nucleotide analogs include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In some embodiments, a 3E10 or 3E10 variant binding domain includes one or more non-canonical adenosine nucleotide analog. Non-limited examples of non-canonical adenosine nucleotide analogs include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In some embodiments, a 3E10 or 3E10 variant binding domain includes one or more non-canonical guanosine nucleotide analog. Non-limited examples of non-canonical guanosine nucleotide analogs include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

### Effector Domain

As outlined above, 3E10 or 3E10 variant binding domains improve binding between a polynucleotide and a 3E10 antibody or variant thereof, or antigen-binding fragment thereof. In some embodiments, a 3E10 or 3E10 variant binding domain is integrated next to or within a second domain, referred herein as an effector domain. In some embodiments, the effector domain provides a therapeutic effect in certain physiological contexts. For instance, in some embodiments, an effector domain encodes a therapeutic polypeptide, targets another polynucleotide, e.g., to silence or edit the other polypeptide, or has enzymatic function itself. In some embodiments, the therapeutic polynucleotide is a linear molecule. In other embodiments, a therapeutic polynucleotide is a circularized molecule.

In some embodiments, an effector domain is a single-stranded polynucleotide, e.g., as illustrated in FIGS. 17A-17C. In some embodiments, the effector domain is a single-stranded polyribonucleotide. In some embodiments, the effector domain is a single-stranded deoxyribonucleotide. In some embodiments, an effector domain is a double-stranded polynucleotide, e.g., as illustrated in FIGS. 17D-17F. In some embodiments, the effector domain is a double-stranded polyribonucleotide. In some embodiments, the effector domain is a double-stranded deoxyribonucleotide.

In some embodiments, a therapeutic polynucleotide has at least one 3E10 or 3E10 variant binding domain and two or more effector domains, e.g., as illustrated in FIGS. 17C and 17F. For example, in some embodiments, a therapeutic polynucleotide has 2, 3, 4, 5, 6, 7, 8, 9, 10, or more variant binding domains. Similarly, in some embodiments, a therapeutic polynucleotide has 2, 3, 4, 5, 6, 7, 8, 9, 10, or more effector domains.

Generally, the size of an effector domain and, therefore, the size of a therapeutic polynucleotide, will be determined in large part by the particular therapeutic methodology employed. For instance, several therapeutic strategies, broadly referred to oligonucleotide therapies, employ relatively short polynucleotide effectors e.g., typically less than 100 nucleotides/base pairs. Examples of these shorter effector molecules include triplex-forming oligodeoxynucleotides (TFOs), Antisense oligodeoxynucleotides (AS-ODNs), immunostimulatory ODNs (CpG-ODNs and IMOs), catalytic oligonucleotides (e.g., ribozymes and DNAzymes), small interfering RNAs (siRNAs), and aptamer oligonucleotides. By contrast, other therapeutic polynucleotide strategies, for example therapies supplementing or replacing loss-of-function genes in a tissue, employ significantly longer effector polynucleotides that can reach several thousand base pairs in length or longer. Examples of these longer effector molecules include DNA vaccines, RNA vaccines, mRNA gene supplementation, DNA gene supplementation, and DNA editing polynucleotides.

Accordingly, in some embodiments, particularly where oligonucleotide therapies are being implemented, an effector domain of a therapeutic polynucleotide is at least 10 nucleotides/base pairs. In some embodiments, an effector domain of a therapeutic polynucleotide is at least 50 nucleotides/base pairs. In some embodiments, an effector domain of a therapeutic polynucleotide is at least 100 nucleotides/base pairs. In some embodiments, an effector domain of a therapeutic polynucleotide is at least about 10, at least about 15, at least about 20, at least about 25, at least about 30, at least about 35, at least about 40, at least about 45, at least about 50, at least about 60, at least about 70, at least about 75, at least about 80, at least about 90, at least about 100, or more nucleotides/base pairs.

Similarly, in some embodiments, particularly where oligonucleotide therapies are being implemented, an effector domain of a therapeutic polynucleotide is no more than 500 nucleotides/base pairs. In some embodiments, an effector domain of a therapeutic polynucleotide is no more than 250 nucleotides/base pairs. In some embodiments, an effector domain of a therapeutic polynucleotide is no more than 100 nucleotides/base pairs. In some embodiments, an effector domain of a therapeutic polynucleotide is no more than about 500, no more than about 450, no more than about 400, no more than about 350, no more than about 300, no more than about 250, no more than about 200, no more than about 150, no more than about 125, no more than about 100, no more than about 90, no more than about 80, no more than about 75, no more than about 70, no more than about 60, no more than about 50, or fewer nucleotides/base pairs.

In some embodiments, particularly where oligonucleotide therapies are being implemented, an effector domain of a therapeutic polynucleotide is from about 10 nucleotides/base pairs to about 500 nucleotides/base pairs. In some embodiments, an effector domain of a therapeutic polynucleotide is from about 10 nucleotides/base pairs to about 250 nucleotides/base pairs. In some embodiments, an effector domain of a therapeutic polynucleotide is from about 10 nucleotides/base pairs to about 100 nucleotides/base pairs.

In some embodiments, particularly where gene supplementation or editing is being implemented, an effector domain of a therapeutic polynucleotide is at least 100 nucleotides/base pairs. In some embodiments, an effector domain of a therapeutic polynucleotide is at least 500 nucleotides/base pairs. In some embodiments, an effector domain of a therapeutic polynucleotide is at least 1000 nucleotides/base pairs. In some embodiments, an effector domain of a therapeutic polynucleotide is at least about 100, at least about 150, at least about 200, at least about 250, at least about 500, at least about 1000, at least about 2000, at least about 3000, at least about 4000, at least about 5000, at least about 10,000, or more nucleotides/base pairs.

Similarly, in some embodiments, particularly where gene therapy, therapeutic mRNA, or gene editing is being implemented, an effector domain of a therapeutic polynucleotide is no more than 10,000 nucleotides/base pairs. In some embodiments, an effector domain of a therapeutic polynucleotide is no more than 7500 nucleotides/base pairs. In some embodiments, an effector domain of a therapeutic polynucleotide is no more than 5000 nucleotides/base pairs. In some embodiments, an effector domain of a therapeutic polynucleotide is no more than about 10,000, no more than about 9000, no more than about 8000, no more than about 7000, no more than about 6000, no more than about 5000, no more than about 4000, no more than about 3000, no more than about 2000, no more than about 1000, or fewer nucleotides/base pairs.

In some embodiments, particularly where gene therapy, therapeutic mRNA, or gene editing is being implemented, an effector domain of a therapeutic polynucleotide is from about 100 nucleotides/base pairs to about 10,000 nucleotides/base pairs. In some embodiments, an effector domain of a therapeutic polynucleotide is from about 500 nucleotides/base pairs to about 10,000 nucleotides/base pairs. In some embodiments, an effector domain of a therapeutic polynucleotide is from about 1000 nucleotides/base pairs to about 5000 nucleotides/base pairs.

In some embodiments, an effector domain includes one or more non-canonical nucleotides, e.g., to improve the stability and/or half-life of the therapeutic polynucleotide in vivo. Examples of non-canonical nucleotides suitable for inclusion in the therapeutic polynucleotide described herein are described in U.S. Pat. No. 9,181,319, the content of which is incorporated herein by reference.

The effector domains described herein are typically therapeutic in nature, as outlined above. Examples of the types of effector domains that find use in the therapeutic polynucleotides of the present disclosure are described in more detail below. However, the effector domains of the therapeutic polynucleotides described herein are not limited to those described below. Rather, a 3E10 or 3E10 variant binding domain can be incorporated in any polynucleotide in order to improve the binding properties of the polynucleotide for a 3E10 antibody or variant thereof, or antigen-binding fragment thereof.

### Vectors

In some embodiments, the effector domain is a vector, e.g., encoding a therapeutic polypeptide and/or functional nucleic acid. As used herein, a “vector” refers to a polynucleotide capable of being replicated within a cell, into which a heterologous sequence can be inserted. In some embodiments, the vector is an expression vectors. In some embodiments, expression vectors include one or more transcriptional control sequences and a heterologous sequence, operably-linked to the transcriptional control sequence, encoding a polynucleotide or therapeutic RNA. A coding sequence is “operably linked” and “under the control” of expression control sequences in a cell when the coding sequence assists to direct an RNA polymerase to transcribe the coding sequence.

Suitable expression vectors include, without limitation, plasmids, cosmids, and recombinant viral genomes or portions derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA).

Accordingly, in some embodiments, a therapeutic polynucleotide includes a 3E10 or 3E10 variant binding domain and an effector domain that includes a vector encoding a polypeptide. Similarly, in some embodiments, compositions are provided that include a therapeutically effective amount of a complex formed between (i) a therapeutic polynucleotide having a 3E10 or 3E10 variant binding domain and an effector domain that includes a vector encoding a polypeptide, and (ii) a 3E10 antibody or variant thereof, or antigen-binding fragment thereof. Similarly, in some embodiments, a method is provided for treating a disorder by administering a composition that includes a therapeutically effective amount of a complex formed between (i) a therapeutic polynucleotide having a 3E10 or 3E10 variant binding domain and an effector domain that includes a vector encoding a polypeptide, and (ii) a 3E10 antibody or variant thereof, or antigen-binding fragment thereof.

In some embodiments, the vector polynucleotide encodes a skeletal muscle protein. Accordingly, in some embodiments, a therapeutic polynucleotide includes a 3E10 or 3E10 variant binding domain and an effector domain that includes a vector encoding a skeletal muscle protein. Similarly, in some embodiments, compositions are provided that include a therapeutically effective amount of a complex formed between (i) a therapeutic polynucleotide having a 3E10 or 3E10 variant binding domain and an effector domain that includes a vector encoding a skeletal muscle protein, and (ii) a 3E10 antibody or variant thereof, or antigen-binding fragment thereof. Similarly, in some embodiments, a method is provided for treating a skeletal muscle disorder by administering a composition that includes a therapeutically effective amount of a complex formed between (i) a therapeutic polynucleotide having a 3E10 or 3E10 variant binding domain and an effector domain that includes a vector encoding a skeletal muscle protein, and (ii) a 3E10 antibody or variant thereof, or antigen-binding fragment thereof.

mRNAs

In some embodiments, the effector domain is an mRNA, e.g., encoding a therapeutic polypeptide. In some embodiments, an mRNA includes one or more chemical structures that promote stability and/or translation efficiency. For example, an mRNA can include one or more of a 5′ cap, a 5′ UTR, a 3′ UTR, and/or a poly(A) tail.

In some embodiments, the mRNA has a cap on the 5′ end, a 3′ poly(A) tail, or a combination thereof which determine ribosome binding, initiation of translation and stability mRNA in the cell. 5′ caps provide stability to RNA molecules. The 5′ cap may, for example, be m7G(5′)ppp(5′)G, m7G(5′)ppp(5′)A, G(5′)ppp(5′)G or G(5′)ppp(5′)A cap analogs, which are all commercially available. The 5′ cap can also be an anti-reverse-cap-analog (ARCA) (Stepinski, et al., RNA, 7:1468-95 (2001)) or any other suitable analog. The 5′ cap can be incorporated using techniques known in the art (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

Additionally, in some embodiments, one or more chemical group is attached to the 3′ end of the mRNA to increase stability. For example, in some embodiments, ATP analogs can be incorporated into a poly(A) tail, to increase the stability of the mRNA. Suitable ATP analogs include, but are not limited to, cordiocipin and 8-azaadenosine.

Accordingly, in some embodiments, a therapeutic polynucleotide includes a 3E10 or 3E10 variant binding domain and an effector domain that includes an mRNA encoding a polypeptide. Similarly, in some embodiments, compositions are provided that include a therapeutically effective amount of a complex formed between (i) a therapeutic polynucleotide having a 3E10 or 3E10 variant binding domain and an effector domain that includes an mRNA encoding a polypeptide, and (ii) a 3E10 antibody or variant thereof, or antigen-binding fragment thereof. Similarly, in some embodiments, a method is provided for treating a disorder by administering a composition that includes a therapeutically effective amount of a complex formed between (i) a therapeutic polynucleotide having a 3E10 or 3E10 variant binding domain and an effector domain that includes an mRNA encoding a polypeptide, and (ii) a 3E10 antibody or variant thereof, or antigen-binding fragment thereof.

In some embodiments, the mRNA encodes a skeletal muscle protein. Accordingly, in some embodiments, a therapeutic polynucleotide includes a 3E10 or 3E10 variant binding domain and an effector domain that includes a vector encoding a skeletal muscle protein. Similarly, in some embodiments, compositions are provided that include a therapeutically effective amount of a complex formed between (i) a therapeutic polynucleotide having a 3E10 or 3E10 variant binding domain and an effector domain that includes an mRNA encoding a skeletal muscle protein, and (ii) a 3E10 antibody or variant thereof, or antigen-binding fragment thereof. Similarly, in some embodiments, a method is provided for treating a skeletal muscle disorder by administering a composition that includes a therapeutically effective amount of a complex formed between (i) a therapeutic polynucleotide having a 3E10 or 3E10 variant binding domain and an effector domain that includes an mRNA encoding a skeletal muscle protein, and (ii) a 3E10 antibody or variant thereof, or antigen-binding fragment thereof.

### Long Non-Coding RNAs (InRNAs)

In some embodiments, the targeting molecule is a long non-coding RNA (lncRNA). LncRNAs are a type of RNA, generally defined as transcripts more than 200 nucleotides that are not translated into protein (Perkel JM (June 2013). “Visiting “noncodarnia””. BioTechniques (paper). 54 (6): 301, 303-4). This arbitrary limit distinguishes long ncRNAs from small non-coding RNAs, such as microRNAs (miRNAs), small interfering RNAs (siRNAs), Piwi-interacting RNAs (piRNAs), small nucleolar RNAs (snoRNAs), and other short RNAs.

### Functional Nucleic Acids

In some embodiments, the effector domain is, or encodes for, a functional nucleic acid. Functional nucleic acids are polynucleotides that directly effect a result, as opposed to polynucleotides that encode for a polypeptide that effects a result. Examples of therapeutic nucleic acids include antisense oligonucleotides, siRNA, miRNA, aptamers, ribozymes, RNAi, external guide sequences, and cyclic dinucleotides.

Functional nucleic acid molecules can interact with many different types of ligands, including small molecules, e.g., metabolites, and macromolecules, e.g., DNA, RNA, polypeptides, and/or carbohydrate chains, to cause a therapeutic effect in vivo. In some embodiments, a functional nucleic acid binds a ligand and sequesters the ligand from participating in a biological function, inhibits the ligand from performing a biological function, promotes a biological function of the ligand, or catalyzes a reaction involving the ligand.

For example, in some embodiments, the effector domain is, or encodes for, an antisense oligonucleotide. In some embodiments, an antisense molecule is designed to interact with a target nucleic acid molecule through canonical or non-canonical base pairing, to promote the destruction of the target molecule through, for example, RNAse H mediated RNA-DNA hybrid degradation. Alternatively, in some embodiments, an oligonucleotide is designed to interrupt a processing function, such as transcription or replication.

Accordingly, in some embodiments, a therapeutic polynucleotide includes a 3E10 or 3E10 variant binding domain and an effector domain that includes, or encodes, an antisense oligonucleotide directed against an mRNA molecule encoding a polypeptide. Similarly, in some embodiments, compositions are provided that include a therapeutically effective amount of a complex formed between (i) a therapeutic polynucleotide having a 3E10 or 3E10 variant binding domain and an effector domain that includes, or encodes, an antisense oligonucleotide directed against an mRNA molecule encoding a polypeptide, and (ii) a 3E10 antibody or variant thereof, or antigen-binding fragment thereof. Similarly, in some embodiments, a method is provided for treating a disorder by administering a composition that includes a therapeutically effective amount of a complex formed between (i) a therapeutic polynucleotide having a 3E10 or 3E10 variant binding domain and an effector domain that includes, or encodes, an antisense oligonucleotide directed against an mRNA molecule encoding a polypeptide, and (ii) a 3E10 antibody or variant thereof, or antigen-binding fragment thereof.

In some embodiments, an antisense oligonucleotide is designed to target an mRNA molecule encoding a mutant skeletal muscle protein. Accordingly, in some embodiments, a therapeutic polynucleotide includes a 3E10 or 3E10 variant binding domain and an effector domain that includes, or encodes, an antisense oligonucleotide directed against an mRNA molecule encoding a skeletal muscle protein. Similarly, in some embodiments, compositions are provided that include a therapeutically effective amount of a complex formed between (i) a therapeutic polynucleotide having a 3E10 or 3E10 variant binding domain and an effector domain that includes, or encodes, an antisense oligonucleotide directed against an mRNA molecule encoding a skeletal muscle protein, and (ii) a 3E10 antibody or variant thereof, or antigen-binding fragment thereof. Similarly, in some embodiments, a method is provided for treating a skeletal muscle disorder by administering a composition that includes a therapeutically effective amount of a complex formed between (i) a therapeutic polynucleotide having a 3E10 or 3E10 variant binding domain and an effector domain that includes, or encodes, an antisense oligonucleotide directed against an mRNA molecule encoding a skeletal muscle protein, and (ii) a 3E10 antibody or variant thereof, or antigen-binding fragment thereof.

In some embodiments, the effector domain is, or encodes for, a short interfering RNA (siRNA) or a short hairpin RNA (shRNA). siRNAs are double-stranded RNA that can induce sequence-specific, post-transcriptional gene silencing, thereby decreasing or even inhibiting gene expression. In one example, a siRNA triggers the specific degradation of homologous RNA molecules, such as mRNAs, within the region of sequence identity between both the siRNA and the target RNA. For example, WO 02/44321 (U.S. Pat. No. 7,056,704) discloses siRNAs capable of sequence-specific degradation of target mRNAs when base-paired with 3′ overhanging ends, herein incorporated by reference for the method of making these siRNAs.

siRNA can be chemically or in vitro-synthesized or can be the result of processing short double-stranded hairpin-like RNAs (shRNAs) intracellularly. siRNA can also be synthesized in vitro using kits such as Ambion's SILENCER® siRNA Construction Kit. Kits for the production of vectors encoding shRNA are available, such as, for example, Imgenex's GENESUPPRESSOR™ Construction Kits and Invitrogen's BLOCK-IT™ inducible RNAi plasmid and lentivirus vectors.

Accordingly, in some embodiments, a therapeutic polynucleotide includes a 3E10 or 3E10 variant binding domain and an effector domain that includes, or encodes, an siRNA or shRNA directed against an mRNA molecule encoding a polypeptide. Similarly, in some embodiments, compositions are provided that include a therapeutically effective amount of a complex formed between (i) a therapeutic polynucleotide having a 3E10 or 3E10 variant binding domain and an effector domain that includes, or encodes, an siRNA or shRNA directed against an mRNA molecule encoding a polypeptide, and (ii) a 3E10 antibody or variant thereof, or antigen-binding fragment thereof. Similarly, in some embodiments, a method is provided for treating a disorder by administering a composition that includes a therapeutically effective amount of a complex formed between (i) a therapeutic polynucleotide having a 3E10 or 3E10 variant binding domain and an effector domain that includes, or encodes, an siRNA or shRNA directed against an mRNA molecule encoding a polypeptide, and (ii) a 3E10 antibody or variant thereof, or antigen-binding fragment thereof.

In some embodiments, an siRNA or shRNA is designed to target an mRNA molecule encoding a mutant skeletal muscle protein. Accordingly, in some embodiments, a therapeutic polynucleotide includes a 3E10 or 3E10 variant binding domain and an effector domain that includes, or encodes, an siRNA or shRNA directed against an mRNA molecule encoding a skeletal muscle protein. Similarly, in some embodiments, compositions are provided that include a therapeutically effective amount of a complex formed between (i) a therapeutic polynucleotide having a 3E10 or 3E10 variant binding domain and an effector domain that includes, or encodes, an siRNA or shRNA directed against an mRNA molecule encoding a skeletal muscle protein, and (ii) a 3E10 antibody or variant thereof, or antigen-binding fragment thereof. Similarly, in some embodiments, a method is provided for treating a skeletal muscle disorder by administering a composition that includes a therapeutically effective amount of a complex formed between (i) a therapeutic polynucleotide having a 3E10 or 3E10 variant binding domain and an effector domain that includes, or encodes, an siRNA or shRNA directed against an mRNA molecule encoding a skeletal muscle protein, and (ii) a 3E10 antibody or variant thereof, or antigen-binding fragment thereof.

In some embodiments, an siRNA or shRNA is designed to target a long non-coding RNA (lncRNA). Accordingly, in some embodiments, a therapeutic polynucleotide includes a 3E10 or 3E10 variant binding domain and an effector domain that includes, or encodes, an siRNA or shRNA directed against a lncRNA. Similarly, in some embodiments, compositions are provided that include a therapeutically effective amount of a complex formed between (i) a therapeutic polynucleotide having a 3E10 or 3E10 variant binding domain and an effector domain that includes, or encodes, an siRNA or shRNA directed against a lncRNA, and (ii) a 3E10 antibody or variant thereof, or antigen-binding fragment thereof. Similarly, in some embodiments, a method is provided for treating a skeletal muscle disorder by administering a composition that includes a therapeutically effective amount of a complex formed between (i) a therapeutic polynucleotide having a 3E10 or 3E10 variant binding domain and an effector domain that includes, or encodes, an siRNA or shRNA directed against a lncRNA, and (ii) a 3E10 antibody or variant thereof, or antigen-binding fragment thereof.

In some embodiments, a therapeutic polynucleotide includes a 3E10 or 3E10 variant binding domain and an effector domain that includes, or encodes, an miRNA directed against an mRNA molecule encoding a polypeptide. Similarly, in some embodiments, compositions are provided that include a therapeutically effective amount of a complex formed between (i) a therapeutic polynucleotide having a 3E10 or 3E10 variant binding domain and an effector domain that includes, or encodes, an miRNA directed against an mRNA molecule encoding a polypeptide, and (ii) a 3E10 antibody or variant thereof, or antigen-binding fragment thereof. Similarly, in some embodiments, a method is provided for treating a disorder by administering a composition that includes a therapeutically effective amount of a complex formed between (i) a therapeutic polynucleotide having a 3E10 or 3E10 variant binding domain and an effector domain that includes, or encodes, an miRNA directed against an mRNA molecule encoding a polypeptide, and (ii) a 3E10 antibody or variant thereof, or antigen-binding fragment thereof.

In some embodiments, an miRNA is designed to target an mRNA molecule encoding a mutant skeletal muscle protein. Accordingly, in some embodiments, a therapeutic polynucleotide includes a 3E10 or 3E10 variant binding domain and an effector domain that includes, or encodes, an miRNA directed against an mRNA molecule encoding a skeletal muscle protein. Similarly, in some embodiments, compositions are provided that include a therapeutically effective amount of a complex formed between (i) a therapeutic polynucleotide having a 3E10 or 3E10 variant binding domain and an effector domain that includes, or encodes, an miRNA directed against an mRNA molecule encoding a skeletal muscle protein, and (ii) a 3E10 antibody or variant thereof, or antigen-binding fragment thereof. Similarly, in some embodiments, a method is provided for treating a skeletal muscle disorder by administering a composition that includes a therapeutically effective amount of a complex formed between (i) a therapeutic polynucleotide having a 3E10 or 3E10 variant binding domain and an effector domain that includes, or encodes, an miRNA directed against an mRNA molecule encoding a skeletal muscle protein, and (ii) a 3E10 antibody or variant thereof, or antigen-binding fragment thereof.

In some embodiments, an miRNA is designed to target a long non-coding RNA (lncRNA). Accordingly, in some embodiments, a therapeutic polynucleotide includes a 3E10 or 3E10 variant binding domain and an effector domain that includes, or encodes, an miRNA directed against a lncRNA. Similarly, in some embodiments, compositions are provided that include a therapeutically effective amount of a complex formed between (i) a therapeutic polynucleotide having a 3E10 or 3E10 variant binding domain and an effector domain that includes, or encodes, an miRNA directed against a lncRNA, and (ii) a 3E10 antibody or variant thereof, or antigen-binding fragment thereof. Similarly, in some embodiments, a method is provided for treating a skeletal muscle disorder by administering a composition that includes a therapeutically effective amount of a complex formed between (i) a therapeutic polynucleotide having a 3E10 or 3E10 variant binding domain and an effector domain that includes, or encodes, an miRNA directed against a lncRNA, and (ii) a 3E10 antibody or variant thereof, or antigen-binding fragment thereof.

In some embodiments, the effector domain is, or encodes for, an aptamer. Aptamers are polynucleotides that fold into a defined tertiary structure in order to specifically bind to a target epitope, much like an antibody. Accordingly, in some embodiments, a therapeutic polynucleotide includes a 3E10 or 3E10 variant binding domain and an effector domain that includes, or encodes, an aptamer. Similarly, in some embodiments, compositions are provided that include a therapeutically effective amount of a complex formed between (i) a therapeutic polynucleotide having a 3E10 or 3E10 variant binding domain and an effector domain that includes, or encodes, an aptamer, and (ii) a 3E10 antibody or variant thereof, or antigen-binding fragment thereof. Similarly, in some embodiments, a method is provided for treating a disorder by administering a composition that includes a therapeutically effective amount of a complex formed between (i) a therapeutic polynucleotide having a 3E10 or 3E10 variant binding domain and an effector domain that includes, or encodes, an aptamer, and (ii) a 3E10 antibody or variant thereof, or antigen-binding fragment thereof.

In some embodiments, an aptamer is designed to bind and/or inhibit a mutant skeletal muscle protein. Accordingly, in some embodiments, a therapeutic polynucleotide includes a 3E10 or 3E10 variant binding domain and an effector domain that includes, or encodes, an aptamer directed against a skeletal muscle protein. Similarly, in some embodiments, compositions are provided that include a therapeutically effective amount of a complex formed between (i) a therapeutic polynucleotide having a 3E10 or 3E10 variant binding domain and an effector domain that includes, or encodes, an aptamer directed against a skeletal muscle protein, and (ii) a 3E10 antibody or variant thereof, or antigen-binding fragment thereof. Similarly, in some embodiments, a method is provided for treating a skeletal muscle disorder by administering a composition that includes a therapeutically effective amount of a complex formed between (i) a therapeutic polynucleotide having a 3E10 or 3E10 variant binding domain and an effector domain that includes, or encodes, an aptamer directed against a skeletal muscle protein, and (ii) a 3E10 antibody or variant thereof, or antigen-binding fragment thereof.

In some embodiments, the effector domain is, or encodes for, a ribosome. Ribozymes are catalytic RNA molecules. In some embodiments, a ribozyme has sequence-specific nucleic acid cleavage activity. Accordingly, in some embodiments, a therapeutic polynucleotide includes a 3E10 or 3E10 variant binding domain and an effector domain that includes, or encodes, a ribozyme directed against an mRNA molecule encoding a polypeptide. Similarly, in some embodiments, compositions are provided that include a therapeutically effective amount of a complex formed between (i) a therapeutic polynucleotide having a 3E10 or 3E10 variant binding domain and an effector domain that includes, or encodes, a ribozyme directed against an mRNA molecule encoding a polypeptide, and (ii) a 3E10 antibody or variant thereof, or antigen-binding fragment thereof. Similarly, in some embodiments, a method is provided for treating a disorder by administering a composition that includes a therapeutically effective amount of a complex formed between (i) a therapeutic polynucleotide having a 3E10 or 3E10 variant binding domain and an effector domain that includes, or encodes, a ribozyme directed against an mRNA molecule encoding a polypeptide, and (ii) a 3E10 antibody or variant thereof, or antigen-binding fragment thereof.

In some embodiments, a ribozyme is designed to target an mRNA molecule encoding a mutant skeletal muscle protein. Accordingly, in some embodiments, a therapeutic polynucleotide includes a 3E10 or 3E10 variant binding domain and an effector domain that includes, or encodes, a ribozyme directed against an mRNA molecule encoding a skeletal muscle protein. Similarly, in some embodiments, compositions are provided that include a therapeutically effective amount of a complex formed between (i) a therapeutic polynucleotide having a 3E10 or 3E10 variant binding domain and an effector domain that includes, or encodes, a ribozyme directed against an mRNA molecule encoding a skeletal muscle protein, and (ii) a 3E10 antibody or variant thereof, or antigen-binding fragment thereof. Similarly, in some embodiments, a method is provided for treating a skeletal muscle disorder by administering a composition that includes a therapeutically effective amount of a complex formed between (i) a therapeutic polynucleotide having a 3E10 or 3E10 variant binding domain and an effector domain that includes, or encodes, a ribozyme directed against an mRNA molecule encoding a skeletal muscle protein, and (ii) a 3E10 antibody or variant thereof, or antigen-binding fragment thereof.

### Codon-skewed Therapeutic Polynucleotides

In another aspect, the present disclosure provides single-stranded therapeutic polynucleotides encoding a therapeutic polypeptide that have been codon-altered in order to bind tighter with a 3E10 antibody or variant thereof, or antigen-binding fragment thereof than the natively-encoded polynucleotide. This is based, at least in part, on the discovery that 3E10 antibodies or variants thereof, or antigen-binding fragments thereof bind to some polynucleotide sequences with greater affinity than they bind to other polynucleotide sequences. Without being bound by theory, this result suggests that the effectiveness of 3E10 and 3E10 variant (e.g., 3E10 (D31N))-based polynucleotide delivery systems would depend, at least in part, on the sequence of the polynucleotide being delivered.

As such, it was contemplated that the effectiveness of such a delivery system could be improved by adjusting the codon-usage of a therapeutic polypeptide to generate a polynucleotide having higher usage of polynucleotides that are bound most tightly to a 3E10 antibody or variant thereof, or antigen-binding fragment thereof, e.g., dT, rl, and/or rG, and/or having a lower usage of polynucleotides that are bound least tightly to a 3E10 antibody or variant thereof, or antigen-binding fragment thereof, e.g., dA or rA. Accordingly, in some embodiments, a therapeutic polynucleotide encoding a therapeutic polypeptide has a first codon-altered nucleotide sequence that a 3E10 antibody or variant thereof, or antigen-binding fragment thereof has a greater affinity for than for a second nucleotide sequence that encodes the therapeutic polypeptide using a same coding sequence for the therapeutic polypeptide as found in a genome for the species of the subject.

Generally, the entire therapeutic polynucleotide need not be codon-altered in this fashion. Rather, it is contemplated that codon-alteration of a single subsequence with the therapeutic polynucleotide is sufficient to improve overall binding between the therapeutic polynucleotide and a 3E10 antibody or variant thereof, or antigen-binding fragment thereof. For instance, example therapeutic polynucleotide 1762 illustrated in FIG. 18 includes a plurality of subsequences, represented as subsequences 1763-1767. The boundaries of these subsequences are not meant to denote any functional or structural relevance, other then that they represent different portions of the therapeutic polynucleotide. Accordingly, in some embodiments, one or more of subsequences 1763-1767 are codon altered to increase the usage of a preferred 3E10 or 3E10 variant nucleotide, e.g., dT, rl, and/or rG, and/or to decrease usage of a non-preferred 3E10 or 3E10 variant nucleotide, e.g., dA or dT. In some embodiments, more than one consecutive subsequence within a therapeutic polynucleotide is codon-altered in this manner (e.g., both sub-regions 1764 and 1766 in therapeutic polynucleotide 1762 are codon altered), to increase binding to a 3E10 antibody or variant thereof, or antigen-binding fragment thereof. In some embodiments, the entirety of a therapeutic polynucleotide is codon-altered in this fashion.

In some embodiments, a codon-altered nucleotide subsequence of an mRNA molecule has a guanine content that is greater than the guanine content of the same nucleotide subsequence in the natively-encoded nucleotide sequence. In some embodiments, the first codon-altered nucleotide subsequence has a guanine content that is at least 10% greater than the guanine content of the same nucleotide subsequence in the natively-encoded polynucleotide. In some embodiments, the first codon-altered nucleotide subsequence has a guanine content that is at least 25% greater than the guanine content of the same nucleotide subsequence in the natively-encoded polynucleotide. In some embodiments, the first codon-altered nucleotide subsequence has a guanine content that is at least 50% greater than the guanine content of the same nucleotide subsequence in the natively-encoded polynucleotide. In some embodiments, the first codon-altered nucleotide subsequence has a guanine content that is at least 100% greater than the guanine content of the same nucleotide subsequence in the natively-encoded polynucleotide. In some embodiments, the first codon-altered nucleotide subsequence has a guanine content that is at least about 10%, at least about 25% greater, at least about 50% greater, at least about 75% greater, at least about 100% greater, at least about 150% greater, at least about 200% greater, at least about 300% greater, or at least about 500% greater, or more, than the guanine content of the same nucleotide subsequence in the natively-encoded polynucleotide.

In some embodiments, a codon-altered nucleotide subsequence of an mRNA molecule incorporates inosine ribonucleotides, e.g., at wobble-codon positions and/or as a substitute for guanine nucleotides. However, the presence of multiple inosine nucleotides in close proximity to each other can significantly disrupt translation efficiency of an mRNA. See, for example, Hoernes TP et al., Nat Commun 9:4865 (2018), which is incorporated herein by reference. Accordingly, while inosine can be incorporated into a codon-altered nucleotide subsequence, inosine nucleotides should generally be placed at least 3, 4, 5, 6, 7 or more nucleotides away from the nearest other inosine residues. But, for example, when used in combination with other preferred ribonucleotides, the inclusion of inosine nucleotides into a codon-altered nucleotide subsequence of an mRNA molecule can improve 3E10 and 3E10 variant binding. Accordingly, in some embodiments, a codon-altered nucleotide subsequence of an mRNA molecule has an inosine ribonucleotide content of at least 1%. In some embodiments, a codon-altered nucleotide subsequence of an mRNA molecule has an inosine ribonucleotide content of at least 2.5%. In some embodiments, a codon-altered nucleotide subsequence of an mRNA molecule has an inosine ribonucleotide content of at least 5%. In some embodiments, a codon-altered nucleotide subsequence of an mRNA molecule has an inosine ribonucleotide content of at least 10%. In some embodiments, a codon-altered nucleotide subsequence of an mRNA molecule has an inosine ribonucleotide content of at least about 1%, at least about 2%, at least about 3%, at least about 4%, at least about 5%, at least about 6%, at least about 7%, at least about 8%, at least about 9%, at least about 10%, at least about 12.5%, at least about 15%, at least about 17.5%, at least about 20%, or more.

In some embodiments, a codon-altered nucleotide subsequence of an mRNA molecule has an adenine content that is less than the adenine content of the same nucleotide subsequence in the natively-encoded nucleotide sequence. In some embodiments, the first codon-altered nucleotide subsequence has an adenine content that is at least 10% less than the adenine content of the same nucleotide subsequence in the natively-encoded polynucleotide. In some embodiments, the first codon-altered nucleotide subsequence has an adenine content that is at least 25% less than the adenine content of the same nucleotide subsequence in the natively-encoded polynucleotide. In some embodiments, the first codon-altered nucleotide subsequence has an adenine content that is at least 50% less than the adenine content of the same nucleotide subsequence in the natively-encoded polynucleotide. In some embodiments, the first codon-altered nucleotide subsequence has an adenine content that is at least 75% less than the adenine content of the same nucleotide subsequence in the natively-encoded polynucleotide. In some embodiments, the first codon-altered nucleotide subsequence has an adenine content that is at least about 10% less, at least about 15% less, at least about 20% less, at least about 25% less, at least about 300% less, at least about 40% less, at least about 50% less, at least about 60% less, at least about 75% less, or at least about 90% less than the adenine content of the same nucleotide subsequence in the natively-encoded polynucleotide.

In some embodiments, a codon-altered nucleotide subsequence of a DNA molecule, e.g., a single-stranded DNA molecule, has a thymine content that is greater than the thymine content of the same nucleotide subsequence in the natively-encoded nucleotide sequence. In some embodiments, the first codon-altered nucleotide subsequence has a thymine content that is at least 10% greater than the thymine content of the same nucleotide subsequence in the natively-encoded polynucleotide. In some embodiments, the first codon-altered nucleotide subsequence has a thymine content that is at least 25% greater than the thymine content of the same nucleotide subsequence in the natively-encoded polynucleotide. In some embodiments, the first codon-altered nucleotide subsequence has a thymine content that is at least 50% greater than the thymine content of the same nucleotide subsequence in the natively-encoded polynucleotide. In some embodiments, the first codon-altered nucleotide subsequence has a thymine content that is at least 100% greater than the thymine content of the same nucleotide subsequence in the natively-encoded polynucleotide. In some embodiments, the first codon-altered nucleotide subsequence has a thymine content that is at least about 10%, at least about 25% greater, at least about 50% greater, at least about 75% greater, at least about 100% greater, at least about 150% greater, at least about 200% greater, at least about 300% greater, or at least about 500% greater, or more, than the thymine content of the same nucleotide subsequence in the natively-encoded polynucleotide.

In some embodiments, a codon-altered nucleotide subsequence a DNA molecule, e.g., a single-stranded DNA molecule, has an adenine content that is less than the adenine content of the same nucleotide subsequence in the natively-encoded nucleotide sequence. In some embodiments, the first codon-altered nucleotide subsequence has an adenine content that is at least 10% less than the adenine content of the same nucleotide subsequence in the natively-encoded polynucleotide. In some embodiments, the first codon-altered nucleotide subsequence has an adenine content that is at least 25% less than the adenine content of the same nucleotide subsequence in the natively-encoded polynucleotide. In some embodiments, the first codon-altered nucleotide subsequence has an adenine content that is at least 50% less than the adenine content of the same nucleotide subsequence in the natively-encoded polynucleotide. In some embodiments, the first codon-altered nucleotide subsequence has an adenine content that is at least 75% less than the adenine content of the same nucleotide subsequence in the natively-encoded polynucleotide. In some embodiments, the first codon-altered nucleotide subsequence has an adenine content that is at least about 10% less, at least about 15% less, at least about 20% less, at least about 25% less, at least about 300% less, at least about 40% less, at least about 50% less, at least about 60% less, at least about 75% less, or at least about 90% less than the adenine content of the same nucleotide subsequence in the natively-encoded polynucleotide.

### Gene Replacement Therapy

Gene replacement therapy refers to a number of therapeutic techniques for delivering a functional copy of a gene to a tissue in need of the protein encoded by the gene, including DNA-based gene therapy techniques in which a functional copy of the gene is transcribed within the cell, e.g., with or without being stably integrated into the genome of the subject, gene editing therapies, such as CRISPR/Cas, that repair or replace mutant copies of the gene or specific nucleotides in the host's genome, and mRNA delivery-based approaches in which mRNA encoding the protein are delivered to the cell, eliminating the need to transcribe an exogenous copy of the gene. Researchers have developed, and continue to develop, gene replacement therapies for a diverse set of disorders, most notably genetic disorders and cancers in a subject has one or two mutant or non-functioning copies of the gene, e.g., due to mutations in the gene that cause partial or complete loss-of-function, mutations in an associated regulatory region that down-regulates gene transcription, and/or small genomic deletions.

The complexes described herein between a therapeutic polynucleotide of the disclosure and a 3E10 antibody or variant thereof, or antigen-binding fragment thereof are particularly well suited for use in gene replacement therapy at least because the 3E10 or 3E10 variant binding domain improves the strength of the binding interaction between the therapeutic polynucleotide and the 3E10 antibody or variant thereof, or antigen-binding fragment thereof. This enhances the advantageous provided by the 3E10 antibody or variant thereof, or antigen-binding fragment thereof, including (i) delivery of the polynucleotide to in vivo tissues following systemic administration, (ii) incorporation of the polynucleotide into cells following localization to the tissue, (iii) and protection of the polynucleotide from extracellular degradation. Further, as opposed to traditional nucleic acid-based therapies that are reliant upon liposomal encapsulation or integration into a recombinant viral vector, the therapeutic nucleotide-3E10 antibody or variant thereof, or antigen-binding fragment thereof complex can be administered in a non-encapsulated form, eliminating the possibility of adverse immunological effects associated with viral capsids and liposomes.

Accordingly, in some embodiments, methods are provided for gene replacement therapy. The methods include parenterally administering a pharmaceutical composition having a therapeutically effective amount of a complex formed between (i) a therapeutic polynucleotide having a 3E10 or 3E10 variant binding domain and an effector domain that encodes a therapeutic polypeptide, and (ii) a 3E10 antibody or variant thereof, or antigen-binding fragment thereof.

Although, in some embodiments, the therapeutic polypeptide encoded by the polynucleotide is a wild-type version of the therapeutic protein, it will be appreciated that naturally occurring variants or engineered versions of a therapeutic protein may also find use in the compositions and methods described herein. For example, it is common for a therapeutic enzyme to be engineered to improve enzymatic activity. Further, in certain instances, where the wild type version of a therapeutic protein is particularly large and/or includes one or more domains that are particularly susceptible to proteolytic degradation, is it common for the polypeptide encoded to be engineered to make the protein smaller and/or to remove susceptible regions that are dispensable for protein function.

Similarly, in some embodiments, methods are provided for gene editing therapy. The methods include parenterally administering a pharmaceutical composition having a therapeutically effective amount of a complex formed between (i) a therapeutic polynucleotide having a 3E10 or 3E10 variant binding domain and an effector domain that encodes a system for gene editing, and (ii) a 3E10 antibody or variant thereof, or antigen-binding fragment thereof. In some embodiments, the system for gene editing comprises a CRISPR/Cas system, e.g., a CRISPR/Cas9 system. In some embodiments, the CRISPR/Cas9 system comprises a guide RNA (gRNA, e.g., a fusion between a crRNA and a tracrRNA) targeting a portion of the gene to be edited and a Cas9 polypeptide. In some embodiments, the gRNA and Cas9 polypeptide are encoded for on a single therapeutic polynucleotide. In other embodiments, the pharmaceutical composition includes a first complex formed using a first therapeutic polynucleotide encoding a gRNA and a second complex formed using a second therapeutic polynucleotide encoding a Cas9 polypeptide.

### 3E10/3E10 Variant-Therapeutic Polynucleotide Compositions

In one aspect, the present disclosure provides pharmaceutical compositions including a complex formed between a therapeutic polynucleotide, e.g., as described above, and a 3E10 antibody or variant thereof, or antigen-binding fragment thereof, as described herein.

In some embodiments, a pharmaceutical composition described herein has a molar ratio of 3E10 antibody or variant thereof, or antigen-binding fragment thereof to therapeutic mRNA of at least 2:1. As reported in Examples 12 and 14, the use of molar ratios of 3E10 antibody or variant thereof, or antigen-binding fragment thereof to therapeutic polynucleotides in the compositions described herein protects the therapeutic polynucleotides from degradation.

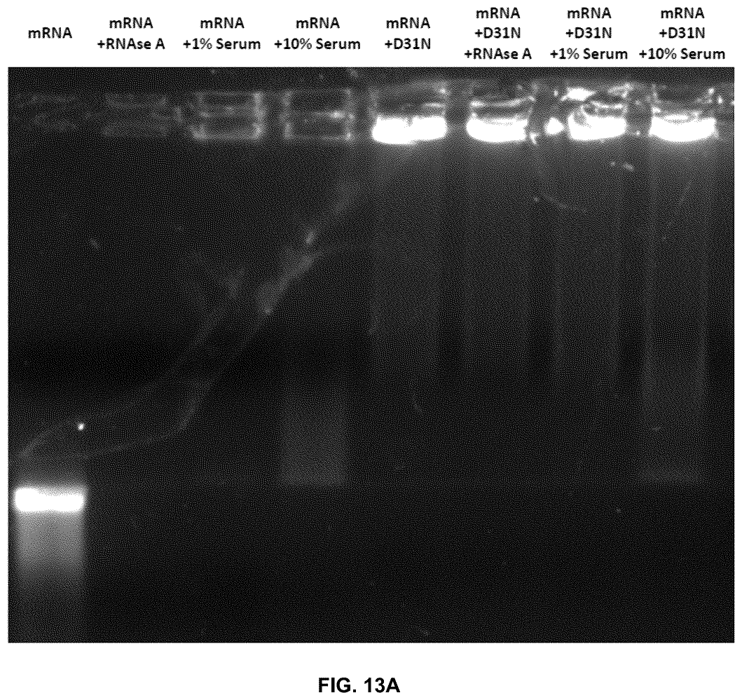

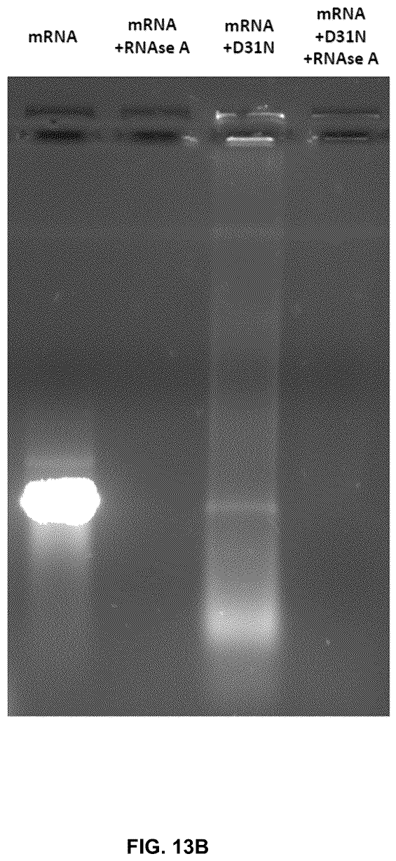

Further, as illustrated in FIGS. 13A and 13B, while parental 3E10 antibodies protected mRNA from RNAse A-mediated RNA degradation at molar ratios of 2:1 and 20:1, the protection afforded by the 20:1 molar ratio exceeded the protection afforded at 2:1. Accordingly, in some embodiments, a pharmaceutical composition described herein has a molar ratio of 3E10 antibody or variant thereof, or antigen-binding fragment thereof to therapeutic polynucleotide that is at least about 2:1. In some embodiments, a pharmaceutical composition described herein has a molar ratio of 3E10 antibody or variant thereof, or antigen-binding fragment thereof to therapeutic polynucleotide that is at least about 5:1. In some embodiments, a pharmaceutical composition described herein has a molar ratio of 3E10 antibody or variant thereof, or antigen-binding fragment thereof to therapeutic polynucleotide that is at least about 7.5:1. In some embodiments, a pharmaceutical composition described herein has a molar ratio of 3E10 antibody or variant thereof, or antigen-binding fragment thereof to therapeutic polynucleotide that is at least about 10:1. In some embodiments, a pharmaceutical composition described herein has a molar ratio of 3E10 antibody or variant thereof, or antigen-binding fragment thereof to therapeutic polynucleotide that is at least about 15:1. In some embodiments, a pharmaceutical composition described herein has a molar ratio of 3E10 antibody or variant thereof, or antigen-binding fragment thereof to therapeutic polynucleotide that is at least about 20:1. In some embodiments, a pharmaceutical composition described herein has a molar ratio of 3E10 antibody or variant thereof, or antigen-binding fragment thereof to therapeutic polynucleotide that is at least about 25:1. In some embodiments, a pharmaceutical composition described herein has a molar ratio of 3E10 antibody or variant thereof, or antigen-binding fragment thereof to therapeutic polynucleotide that is at least about 30:1. In some embodiments, a pharmaceutical composition described herein has a molar ratio of 3E10 antibody or variant thereof, or antigen-binding fragment thereof to therapeutic polynucleotide that is at least about 40:1. In some embodiments, a pharmaceutical composition described herein has a molar ratio of 3E10 antibody or variant thereof, or antigen-binding fragment thereof to therapeutic polynucleotide that is at least about 50:1.

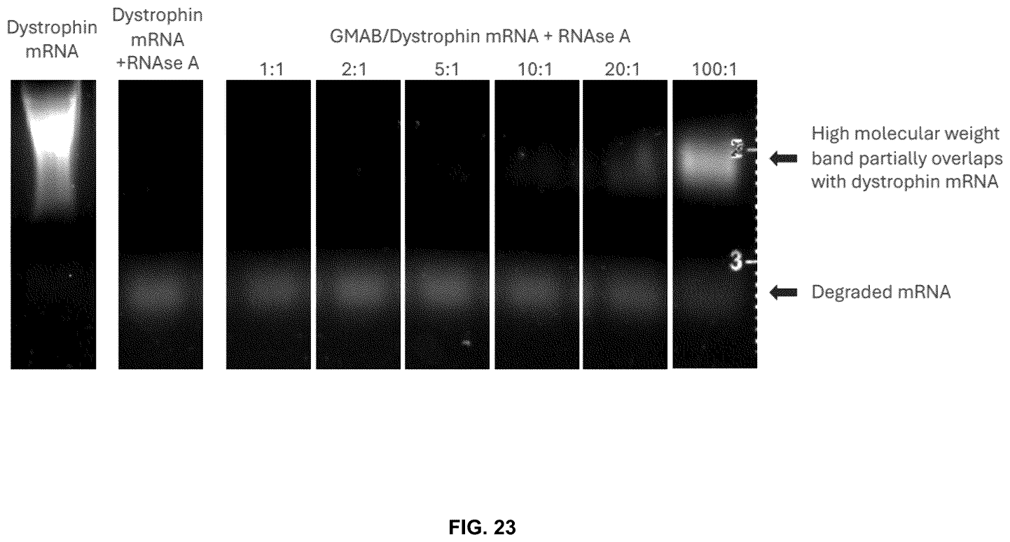

Further, as shown in FIG. 23, the use of higher stoichiometric ratios better protect longer polynucleotides from degradation. Accordingly, in some embodiments, a pharmaceutical composition described herein has a molar ratio of 3E10 antibody or variant thereof, or antigen-binding fragment thereof to therapeutic polynucleotide that is at least about 50:1. In some embodiments, a pharmaceutical composition described herein has a molar ratio of 3E10 antibody or variant thereof, or antigen-binding fragment thereof to therapeutic polynucleotide that is at least about 75:1. In some embodiments, a pharmaceutical composition described herein has a molar ratio of 3E10 antibody or variant thereof, or antigen-binding fragment thereof to therapeutic polynucleotide that is at least about 100:1. In some embodiments, a pharmaceutical composition described herein has a molar ratio of 3E10 antibody or variant thereof, or antigen-binding fragment thereof to therapeutic polynucleotide that is at least about 125:1. In some embodiments, a pharmaceutical composition described herein has a molar ratio of 3E10 antibody or variant thereof, or antigen-binding fragment thereof to therapeutic polynucleotide that is at least about 150:1. In some embodiments, a pharmaceutical composition described herein has a molar ratio of 3E10 antibody or variant thereof, or antigen-binding fragment thereof to therapeutic polynucleotide that is at least about 200:1. In some embodiments, a longer polynucleotide is at least 1000 nucleotides in length, e.g., 1000 nucleotides for a single-stranded polynucleotide or 1000 base pairs for a double-stranded polynucleotide. In some embodiments, a longer polynucleotide is at least 1500 nucleotides in length. In some embodiments, a longer polynucleotide is at least 2000 nucleotides in length. In some embodiments, a longer polynucleotide is at least 2500 nucleotides in length. In some embodiments, a longer polynucleotide is at least 3000 nucleotides in length. In some embodiments, a longer polynucleotide is at least 4000 nucleotides in length. In some embodiments, a longer polynucleotide is at least 5000 nucleotides in length. In some embodiments, a longer polynucleotide is at least 7500 nucleotides in length. In some embodiments, a longer polynucleotide is at least 10,000 nucleotides in length.

In some embodiments, a pharmaceutical composition described herein has a molar ratio of 3E10 antibody or variant thereof, or antigen-binding fragment thereof to therapeutic polynucleotide that is at least about 3:1, at least about 4:1, at least about 5:1, at least about 6:1, at least about 7:1, at least about 8:1, at least about 9:1, at least about 10:1, at least about 11:1, at least about 12:1, at least about 13:1, at least about 14:1, at least about 15:1, at least about 16:1, at least about 17:1, at least about 18:1, at least about 19:1, at least about 20:1, at least about 21:1, at least about 22:1, at least about 23:1, at least about 24:1, at least about 25:1, at least about 26:1, at least about 27:1, at least about 28:1, at least about 29:1, at least about 30:1, at least about 31:1, at least about 32:1, at least about 33:1, at least about 34: 1, at least about 35:1, at least about 36:1, at least about 37:1, at least about 38:1, at least about 39: 1, at least about 40: 1, at least about 41:1, at least about 42: 1, at least about 43:1, at least about 44: 1, at least about 45:1, at least about 50:1, at least about 55:1, at least about 60:1, at least about 70:1, at least about 75:1, at least about 80:1, at least about 85:1, at least about 90:1, at least about 95:1, at least about 100:1, at least about 110:1, at least about 120:1, at least about 125:1, at least about 130:1, at least about 140: 1, at least about 150:1, at least about 160:1, at least about 170:1, at least about 175:1, at least about 180:1, at least about 190:1, at least about 200:1, or greater.

In some embodiments, a pharmaceutical composition described herein has a molar ratio of 3E10 antibody or variant thereof, or antigen-binding fragment thereof to therapeutic polynucleotide that is at least 2:1. In some embodiments, a pharmaceutical composition described herein has a molar ratio of 3E10 antibody or variant thereof, or antigen-binding fragment thereof to therapeutic polynucleotide that is at least 5:1. In some embodiments, a pharmaceutical composition described herein has a molar ratio of 3E10 antibody or variant thereof, or antigen-binding fragment thereof to therapeutic polynucleotide that is at least 7.5:1. In some embodiments, a pharmaceutical composition described herein has a molar ratio of 3E10 antibody or variant thereof, or antigen-binding fragment thereof to therapeutic polynucleotide that is at least 10:1. In some embodiments, a pharmaceutical composition described herein has a molar ratio of 3E10 antibody or variant thereof, or antigen-binding fragment thereof to therapeutic polynucleotide that is at least 15:1. In some embodiments, a pharmaceutical composition described herein has a molar ratio of 3E10 antibody or variant thereof, or antigen-binding fragment thereof to therapeutic polynucleotide that is at least 20:1. In some embodiments, a pharmaceutical composition described herein has a molar ratio of 3E10 antibody or variant thereof, or antigen-binding fragment thereof to therapeutic polynucleotide that is at least 25:1. In some embodiments, a pharmaceutical composition described herein has a molar ratio of 3E10 antibody or variant thereof, or antigen-binding fragment thereof to therapeutic polynucleotide that is at least 30:1. In some embodiments, a pharmaceutical composition described herein has a molar ratio of 3E10 antibody or variant thereof, or antigen-binding fragment thereof to therapeutic polynucleotide that is at least 40:1. In some embodiments, a pharmaceutical composition described herein has a molar ratio of 3E10 antibody or variant thereof, or antigen-binding fragment thereof to therapeutic polynucleotide that is at least 50:1. In some embodiments, a pharmaceutical composition described herein has a molar ratio of 3E10 antibody or variant thereof, or antigen-binding fragment thereof to therapeutic polynucleotide that is at least 75:1. In some embodiments, a pharmaceutical composition described herein has a molar ratio of 3E10 antibody or variant thereof, or antigen-binding fragment thereof to therapeutic polynucleotide that is at least 100:1. In some embodiments, a pharmaceutical composition described herein has a molar ratio of 3E10 antibody or variant thereof, or antigen-binding fragment thereof to therapeutic polynucleotide that is at least 125:1. In some embodiments, a pharmaceutical composition described herein has a molar ratio of 3E10 antibody or variant thereof, or antigen-binding fragment thereof to therapeutic polynucleotide that is at least 150:1. In some embodiments, a pharmaceutical composition described herein has a molar ratio of 3E10 antibody or variant thereof, or antigen-binding fragment thereof to therapeutic polynucleotide that is at least 200:1.

In some embodiments, a pharmaceutical composition described herein has a molar ratio of 3E10 antibody or variant thereof, or antigen-binding fragment thereof to therapeutic polynucleotide that is at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, at least 16:1, at least 17:1, at least 18:1, at least 19:1, at least 20:1, at least 21:1, at least 22:1, at least 23:1, at least 24:1, at least 25:1, at least 26:1, at least 27:1, at least 28:1, at least 29:1, at least 30:1, at least 31:1, at least 32: 1, at least 33:1, at least 34:1, at least 35:1, at least 36:1, at least 37:1, at least 38: 1, at least 39:1, at least 40:1, at least 41: 1, at least 42:1, at least 43:1, at least 44:1, at least 45:1, at least 50:1, at least 55:1, at least 60: 1, at least 70:1, at least 75:1, at least 80:1, at least 85:1, at least 90:1, at least 95:1, at least 100:1, at least 110:1, at least 120:1, at least 125:1, at least 130:1, at least 140:1, at least 150:1, at least 160: 1, at least 170:1, at least 175:1, at least 180:1, at least 190:1, at least 200:1, or greater.

In some embodiments, a pharmaceutical composition described herein has a molar ratio of 3E10 antibody or variant thereof, or antigen-binding fragment thereof to therapeutic polynucleotide that is 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28: 1, 29:1, 30:1, 31: 1, 32: 1, 33:1, 34:1, 35:1, 36:1, 37:1, 38:1, 39: 1, 40: 1, 41: 1, 42:1, 43:1, 44: 1, 45:1, 50:1, 55:1, 60:1, 70:1, 75:1, 80:1, 85:1, 90:1, 95:1, 100:1, 110:1, 120:1, 125:1, 130:1, 140:1, 150:1, 160:1, 170:1, 175:1, 180:1, 190:1, 200:1, or greater.

In some embodiments, a pharmaceutical composition described herein has a molar ratio of 3E10 antibody or variant thereof, or antigen-binding fragment thereof to therapeutic polynucleotide that is no more than about 200:1. In some embodiments, a pharmaceutical composition described herein has a molar ratio of 3E10 antibody or variant thereof, or antigen-binding fragment thereof to therapeutic polynucleotide that is no more than about 150:1. In some embodiments, a pharmaceutical composition described herein has a molar ratio of 3E10 antibody or variant thereof, or antigen-binding fragment thereof to therapeutic polynucleotide that is no more than about 100:1. In some embodiments, a pharmaceutical composition described herein has a molar ratio of 3E10 antibody or variant thereof, or antigen-binding fragment thereof to therapeutic polynucleotide that is no more than about 50:1. In some embodiments, a pharmaceutical composition described herein has a molar ratio of 3E10 antibody or variant thereof, or antigen-binding fragment thereof to therapeutic polynucleotide that is no more than about 40:1. In some embodiments, a pharmaceutical composition described herein has a molar ratio of 3E10 antibody or variant thereof, or antigen-binding fragment thereof to therapeutic polynucleotide that is no more than about 30:1. In some embodiments, a pharmaceutical composition described herein has a molar ratio of 3E10 antibody or variant thereof, or antigen-binding fragment thereof to therapeutic polynucleotide that is no more than about 25:1. In some embodiments, a pharmaceutical composition described herein has a molar ratio of 3E10 antibody or variant thereof, or antigen-binding fragment thereof to therapeutic polynucleotide that is no more than about 20:1. In some embodiments, a pharmaceutical composition described herein has a molar ratio of 3E10 antibody or variant thereof, or antigen-binding fragment thereof to therapeutic polynucleotide that is no more than about 15:1. In some embodiments, a pharmaceutical composition described herein has a molar ratio of 3E10 antibody or variant thereof, or antigen-binding fragment thereof to therapeutic polynucleotide that is no more than about 10:1.

In some embodiments, a pharmaceutical composition described herein has a molar ratio of 3E10 antibody or variant thereof, or antigen-binding fragment thereof to therapeutic polynucleotide that is no more than about 200:1, no more than about 175:1, no more than about 150:1, no more than about 125:1, no more than about 100:1, no more than about 75:1, no more than about 50:1, no more than about 45:1, no more than about 40:1, no more than about 35:1, no more than about 30:1, no more than about 35:1, no more than about 30:1, no more than about 25:1, no more than about 20:1, or less.

In some embodiments, a pharmaceutical composition described herein has a molar ratio of 3E10 antibody or variant thereof, or antigen-binding fragment thereof to therapeutic polynucleotide that is no more than 200:1. In some embodiments, a pharmaceutical composition described herein has a molar ratio of 3E10 antibody or variant thereof, or antigen-binding fragment thereof to therapeutic polynucleotide that is no more than 150:1. In some embodiments, a pharmaceutical composition described herein has a molar ratio of 3E10 antibody or variant thereof, or antigen-binding fragment thereof to therapeutic polynucleotide that is no more than 100:1. In some embodiments, a pharmaceutical composition described herein has a molar ratio of 3E10 antibody or variant thereof, or antigen-binding fragment thereof to therapeutic polynucleotide that is no more than 50:1. In some embodiments, a pharmaceutical composition described herein has a molar ratio of 3E10 antibody or variant thereof, or antigen-binding fragment thereof to therapeutic polynucleotide that is no more than 40:1. In some embodiments, a pharmaceutical composition described herein has a molar ratio of 3E10 antibody or variant thereof, or antigen-binding fragment thereof to therapeutic polynucleotide that is no more than 30:1. In some embodiments, a pharmaceutical composition described herein has a molar ratio of 3E10 antibody or variant thereof, or antigen-binding fragment thereof to therapeutic polynucleotide that is no more than 25:1. In some embodiments, a pharmaceutical composition described herein has a molar ratio of 3E10 antibody or variant thereof, or antigen-binding fragment thereof to therapeutic polynucleotide that is no more than 20:1. In some embodiments, a pharmaceutical composition described herein has a molar ratio of 3E10 antibody or variant thereof, or antigen-binding fragment thereof to therapeutic polynucleotide that is no more than 15:1. In some embodiments, a pharmaceutical composition described herein has a molar ratio of 3E10 antibody or variant thereof, or antigen-binding fragment thereof to therapeutic polynucleotide that is no more than 10:1.

In some embodiments, a pharmaceutical composition described herein has a molar ratio of 3E10 antibody or variant thereof, or antigen-binding fragment thereof to therapeutic polynucleotide that is no more than 200:1, no more than 175:1, no more than 150:1, no more than 125:1, no more than 100:1, no more than 75:1, no more than 50:1, no more than 45:1, no more than 40:1, no more than 35:1, no more than 30:1, no more than 35:1, no more than 30:1, no more than 25:1, no more than 20:1, or less.

In some embodiments, a pharmaceutical composition described herein has a molar ratio of 3E10 antibody or variant thereof, or antigen-binding fragment thereof to therapeutic polynucleotide that is of from 2:1 to 200:1. In some embodiments, a pharmaceutical composition described herein has a molar ratio of 3E10 antibody or variant thereof, or antigen-binding fragment thereof to therapeutic polynucleotide that is of from 2:1 to 175:1. In some embodiments, a pharmaceutical composition described herein has a molar ratio of 3E10 antibody or variant thereof, or antigen-binding fragment thereof to therapeutic polynucleotide that is of from 2:1 to 150:1. In some embodiments, a pharmaceutical composition described herein has a molar ratio of 3E10 antibody or variant thereof, or antigen-binding fragment thereof to therapeutic polynucleotide that is of from 2:1 to 125:1. In some embodiments, a pharmaceutical composition described herein has a molar ratio of 3E10 antibody or variant thereof, or antigen-binding fragment thereof to therapeutic polynucleotide that is of from 2:1 to 100:1. In some embodiments, a pharmaceutical composition described herein has a molar ratio of 3E10 antibody or variant thereof, or antigen-binding fragment thereof to therapeutic polynucleotide that is of from 2:1 to 75:1. In some embodiments, a pharmaceutical composition described herein has a molar ratio of 3E10 antibody or variant thereof, or antigen-binding fragment thereof to therapeutic polynucleotide that is of from 2:1 to 50:1. In some embodiments, a pharmaceutical composition described herein has a molar ratio of 3E10 antibody or variant thereof, or antigen-binding fragment thereof to therapeutic polynucleotide that is of from 2:1 to 40:1. In some embodiments, a pharmaceutical composition described herein has a molar ratio of 3E10 antibody or variant thereof, or antigen-binding fragment thereof to therapeutic polynucleotide that is of from 2:1 to 30:1. In some embodiments, a pharmaceutical composition described herein has a molar ratio of 3E10 antibody or variant thereof, or antigen-binding fragment thereof to therapeutic polynucleotide that is of from 2:1 to 25:1. In some embodiments, a pharmaceutical composition described herein has a molar ratio of 3E10 antibody or variant thereof, or antigen-binding fragment thereof to therapeutic polynucleotide that is of from 2:1 to 20:1. In some embodiments, a pharmaceutical composition described herein has a molar ratio of 3E10 antibody or variant thereof, or antigen-binding fragment thereof to therapeutic polynucleotide that is of from 2:1 to 15:1. In some embodiments, a pharmaceutical composition described herein has a molar ratio of 3E10 antibody or variant thereof, or antigen-binding fragment thereof to therapeutic polynucleotide that is of from 2:1 to 10:1. In some embodiments, a pharmaceutical composition described herein has a molar ratio of 3E10 antibody or variant thereof, or antigen-binding fragment thereof to therapeutic polynucleotide that is of from 2:1 to 7.5:1. In some embodiments, a pharmaceutical composition described herein has a molar ratio of 3E10 antibody or variant thereof, or antigen-binding fragment thereof to therapeutic polynucleotide that is of from 2:1 to 5:1. In some embodiments, a pharmaceutical composition described herein has a molar ratio of 3E10 antibody or variant thereof, or antigen-binding fragment thereof to therapeutic polynucleotide that is of from 2:1 to 3:1.

In some embodiments, a pharmaceutical composition described herein has a molar ratio of 3E10 antibody or variant thereof, or antigen-binding fragment thereof to therapeutic polynucleotide that is of from 3:1 to 200:1. In some embodiments, a pharmaceutical composition described herein has a molar ratio of 3E10 antibody or variant thereof, or antigen-binding fragment thereof to therapeutic polynucleotide that is of from 3:1 to 175:1. In some embodiments, a pharmaceutical composition described herein has a molar ratio of 3E10 antibody or variant thereof, or antigen-binding fragment thereof to therapeutic polynucleotide that is of from 3:1 to 150:1. In some embodiments, a pharmaceutical composition described herein has a molar ratio of 3E10 antibody or variant thereof, or antigen-binding fragment thereof to therapeutic polynucleotide that is of from 3:1 to 125:1. In some embodiments, a pharmaceutical composition described herein has a molar ratio of 3E10 antibody or variant thereof, or antigen-binding fragment thereof to therapeutic polynucleotide that is of from 3:1 to 100:1. In some embodiments, a pharmaceutical composition described herein has a molar ratio of 3E10 antibody or variant thereof, or antigen-binding fragment thereof to therapeutic polynucleotide that is of from 3:1 to 75:1. In some embodiments, a pharmaceutical composition described herein has a molar ratio of 3E10 antibody or variant thereof, or antigen-binding fragment thereof to therapeutic polynucleotide that is of from 3:1 to 50:1. In some embodiments, a pharmaceutical composition described herein has a molar ratio of 3E10 antibody or variant thereof, or antigen-binding fragment thereof to therapeutic polynucleotide that is of from 3:1 to 40:1. In some embodiments, a pharmaceutical composition described herein has a molar ratio of 3E10 antibody or variant thereof, or antigen-binding fragment thereof to therapeutic polynucleotide that is of from 3:1 to 30:1. In some embodiments, a pharmaceutical composition described herein has a molar ratio of 3E10 antibody or variant thereof, or antigen-binding fragment thereof to therapeutic polynucleotide that is of from 3:1 to 25:1. In some embodiments, a pharmaceutical composition described herein has a molar ratio of 3E10 antibody or variant thereof, or antigen-binding fragment thereof to therapeutic polynucleotide that is of from 3:1 to 20:1. In some embodiments, a pharmaceutical composition described herein has a molar ratio of 3E10 antibody or variant thereof, or antigen-binding fragment thereof to therapeutic polynucleotide that is of from 3:1 to 15:1. In some embodiments, a pharmaceutical composition described herein has a molar ratio of 3E10 antibody or variant thereof, or antigen-binding fragment thereof to therapeutic polynucleotide that is of from 3:1 to 10:1. In some embodiments, a pharmaceutical composition described herein has a molar ratio of 3E10 antibody or variant thereof, or antigen-binding fragment thereof to therapeutic polynucleotide that is of from 3:1 to 7.5:1. In some embodiments, a pharmaceutical composition described herein has a molar ratio of 3E10 antibody or variant thereof, or antigen-binding fragment thereof to therapeutic polynucleotide that is of from 3:1 to 5:1.

In some embodiments, a pharmaceutical composition described herein has a molar ratio of 3E10 antibody or variant thereof, or antigen-binding fragment thereof to therapeutic polynucleotide that is of from 5:1 to 200:1. In some embodiments, a pharmaceutical composition described herein has a molar ratio of 3E10 antibody or variant thereof, or antigen-binding fragment thereof to therapeutic polynucleotide that is of from 5:1 to 175:1. In some embodiments, a pharmaceutical composition described herein has a molar ratio of 3E10 antibody or variant thereof, or antigen-binding fragment thereof to therapeutic polynucleotide that is of from 5:1 to 150:1. In some embodiments, a pharmaceutical composition described herein has a molar ratio of 3E10 antibody or variant thereof, or antigen-binding fragment thereof to therapeutic polynucleotide that is of from 5:1 to 125:1. In some embodiments, a pharmaceutical composition described herein has a molar ratio of 3E10 antibody or variant thereof, or antigen-binding fragment thereof to therapeutic polynucleotide that is of from 5:1 to 100:1. In some embodiments, a pharmaceutical composition described herein has a molar ratio of 3E10 antibody or variant thereof, or antigen-binding fragment thereof to therapeutic polynucleotide that is of from 5:1 to 75:1. In some embodiments, a pharmaceutical composition described herein has a molar ratio of 3E10 antibody or variant thereof, or antigen-binding fragment thereof to therapeutic polynucleotide that is of from 5:1 to 50:1. In some embodiments, a pharmaceutical composition described herein has a molar ratio of 3E10 antibody or variant thereof, or antigen-binding fragment thereof to therapeutic polynucleotide that is of from 5:1 to 40:1. In some embodiments, a pharmaceutical composition described herein has a molar ratio of 3E10 antibody or variant thereof, or antigen-binding fragment thereof to therapeutic polynucleotide that is of from 5:1 to 30:1. In some embodiments, a pharmaceutical composition described herein has a molar ratio of 3E10 antibody or variant thereof, or antigen-binding fragment thereof to therapeutic polynucleotide that is of from 5:1 to 25:1. In some embodiments, a pharmaceutical composition described herein has a molar ratio of 3E10 antibody or variant thereof, or antigen-binding fragment thereof to therapeutic polynucleotide that is of from 5:1 to 20:1. In some embodiments, a pharmaceutical composition described herein has a molar ratio of 3E10 antibody or variant thereof, or antigen-binding fragment thereof to therapeutic polynucleotide that is of from 5:1 to 15:1. In some embodiments, a pharmaceutical composition described herein has a molar ratio of 3E10 antibody or variant thereof, or antigen-binding fragment thereof to therapeutic polynucleotide that is of from 5:1 to 10:1. In some embodiments, a pharmaceutical composition described herein has a molar ratio of 3E10 antibody or variant thereof, or antigen-binding fragment thereof to therapeutic polynucleotide that is of from 5:1 to 7.5:1.

In some embodiments, a pharmaceutical composition described herein has a molar ratio of 3E10 antibody or variant thereof, or antigen-binding fragment thereof to therapeutic polynucleotide that is of from 7.5:1 to 200:1. In some embodiments, a pharmaceutical composition described herein has a molar ratio of 3E10 antibody or variant thereof, or antigen-binding fragment thereof to therapeutic polynucleotide that is of from 7.5:1 to 175:1. In some embodiments, a pharmaceutical composition described herein has a molar ratio of 3E10 antibody or variant thereof, or antigen-binding fragment thereof to therapeutic polynucleotide that is of from 7.5:1 to 150:1. In some embodiments, a pharmaceutical composition described herein has a molar ratio of 3E10 antibody or variant thereof, or antigen-binding fragment thereof to therapeutic polynucleotide that is of from 7.5:1 to 125:1. In some embodiments, a pharmaceutical composition described herein has a molar ratio of 3E10 antibody or variant thereof, or antigen-binding fragment thereof to therapeutic polynucleotide that is of from 7.5:1 to 100:1. In some embodiments, a pharmaceutical composition described herein has a molar ratio of 3E10 antibody or variant thereof, or antigen-binding fragment thereof to therapeutic polynucleotide that is of from 7.5:1 to 75:1. In some embodiments, a pharmaceutical composition described herein has a molar ratio of 3E10 antibody or variant thereof, or antigen-binding fragment thereof to therapeutic polynucleotide that is of from 7.5:1 to 50:1. In some embodiments, a pharmaceutical composition described herein has a molar ratio of 3E10 antibody or variant thereof, or antigen-binding fragment thereof to therapeutic polynucleotide that is of from 7.5:1 to 40:1. In some embodiments, a pharmaceutical composition described herein has a molar ratio of 3E10 antibody or variant thereof, or antigen-binding fragment thereof to therapeutic polynucleotide that is of from 7.5:1 to 30:1. In some embodiments, a pharmaceutical composition described herein has a molar ratio of 3E10 antibody or variant thereof, or antigen-binding fragment thereof to therapeutic polynucleotide that is of from 7.5:1 to 25:1. In some embodiments, a pharmaceutical composition described herein has a molar ratio of 3E10 antibody or variant thereof, or antigen-binding fragment thereof to therapeutic polynucleotide that is of from 7.5:1 to 20:1. In some embodiments, a pharmaceutical composition described herein has a molar ratio of 3E10 antibody or variant thereof, or antigen-binding fragment thereof to therapeutic polynucleotide that is of from 7.5:1 to 15:1. In some embodiments, a pharmaceutical composition described herein has a molar ratio of 3E10 antibody or variant thereof, or antigen-binding fragment thereof to therapeutic polynucleotide that is of from 7.5:1 to 10:1.

In some embodiments, a pharmaceutical composition described herein has a molar ratio of 3E10 antibody or variant thereof, or antigen-binding fragment thereof to therapeutic polynucleotide that is of from 10:1 to 200:1. In some embodiments, a pharmaceutical composition described herein has a molar ratio of 3E10 antibody or variant thereof, or antigen-binding fragment thereof to therapeutic polynucleotide that is of from 10:1 to 175:1. In some embodiments, a pharmaceutical composition described herein has a molar ratio of 3E10 antibody or variant thereof, or antigen-binding fragment thereof to therapeutic polynucleotide that is of from 10:1 to 150:1. In some embodiments, a pharmaceutical composition described herein has a molar ratio of 3E10 antibody or variant thereof, or antigen-binding fragment thereof to therapeutic polynucleotide that is of from 10:1 to 125:1. In some embodiments, a pharmaceutical composition described herein has a molar ratio of 3E10 antibody or variant thereof, or antigen-binding fragment thereof to therapeutic polynucleotide that is of from 10:1 to 100:1. In some embodiments, a pharmaceutical composition described herein has a molar ratio of 3E10 antibody or variant thereof, or antigen-binding fragment thereof to therapeutic polynucleotide that is of from 10:1 to 75:1. In some embodiments, a pharmaceutical composition described herein has a molar ratio of 3E10 antibody or variant thereof, or antigen-binding fragment thereof to therapeutic polynucleotide that is of from 10:1 to 50:1. In some embodiments, a pharmaceutical composition described herein has a molar ratio of 3E10 antibody or variant thereof, or antigen-binding fragment thereof to therapeutic polynucleotide that is of from 10:1 to 40:1. In some embodiments, a pharmaceutical composition described herein has a molar ratio of 3E10 antibody or variant thereof, or antigen-binding fragment thereof to therapeutic polynucleotide that is of from 10:1 to 30:1. In some embodiments, a pharmaceutical composition described herein has a molar ratio of 3E10 antibody or variant thereof, or antigen-binding fragment thereof to therapeutic polynucleotide that is of from 10:1 to 25:1. In some embodiments, a pharmaceutical composition described herein has a molar ratio of 3E10 antibody or variant thereof, or antigen-binding fragment thereof to therapeutic polynucleotide that is of from 10:1 to 20:1. In some embodiments, a pharmaceutical composition described herein has a molar ratio of 3E10 antibody or variant thereof, or antigen-binding fragment thereof to therapeutic polynucleotide that is of from 10:1 to 15:1.

In some embodiments, a pharmaceutical composition described herein has a molar ratio of 3E10 antibody or variant thereof, or antigen-binding fragment thereof to therapeutic polynucleotide that is of from 15:1 to 200:1. In some embodiments, a pharmaceutical composition described herein has a molar ratio of 3E10 antibody or variant thereof, or antigen-binding fragment thereof to therapeutic polynucleotide that is of from 15:1 to 175:1. In some embodiments, a pharmaceutical composition described herein has a molar ratio of 3E10 antibody or variant thereof, or antigen-binding fragment thereof to therapeutic polynucleotide that is of from 15:1 to 150:1. In some embodiments, a pharmaceutical composition described herein has a molar ratio of 3E10 antibody or variant thereof, or antigen-binding fragment thereof to therapeutic polynucleotide that is of from 15:1 to 125:1. In some embodiments, a pharmaceutical composition described herein has a molar ratio of 3E10 antibody or variant thereof, or antigen-binding fragment thereof to therapeutic polynucleotide that is of from 15:1 to 100:1. In some embodiments, a pharmaceutical composition described herein has a molar ratio of 3E10 antibody or variant thereof, or antigen-binding fragment thereof to therapeutic polynucleotide that is of from 15:1 to 75:1. In some embodiments, a pharmaceutical composition described herein has a molar ratio of 3E10 antibody or variant thereof, or antigen-binding fragment thereof to therapeutic polynucleotide that is of from 15:1 to 50:1. In some embodiments, a pharmaceutical composition described herein has a molar ratio of 3E10 antibody or variant thereof, or antigen-binding fragment thereof to therapeutic polynucleotide that is of from 15:1 to 40:1. In some embodiments, a pharmaceutical composition described herein has a molar ratio of 3E10 antibody or variant thereof, or antigen-binding fragment thereof to therapeutic polynucleotide that is of from 15:1 to 30:1. In some embodiments, a pharmaceutical composition described herein has a molar ratio of 3E10 antibody or variant thereof, or antigen-binding fragment thereof to therapeutic polynucleotide that is of from 15:1 to 25:1. In some embodiments, a pharmaceutical composition described herein has a molar ratio of 3E10 antibody or variant thereof, or antigen-binding fragment thereof to therapeutic polynucleotide that is of from 15:1 to 20:1.

In some embodiments, a pharmaceutical composition described herein has a molar ratio of 3E10 antibody or variant thereof, or antigen-binding fragment thereof to therapeutic polynucleotide that is of from 20:1 to 200:1. In some embodiments, a pharmaceutical composition described herein has a molar ratio of 3E10 antibody or variant thereof, or antigen-binding fragment thereof to therapeutic polynucleotide that is of from 20:1 to 175:1. In some embodiments, a pharmaceutical composition described herein has a molar ratio of 3E10 antibody or variant thereof, or antigen-binding fragment thereof to therapeutic polynucleotide that is of from 20:1 to 150:1. In some embodiments, a pharmaceutical composition described herein has a molar ratio of 3E10 antibody or variant thereof, or antigen-binding fragment thereof to therapeutic polynucleotide that is of from 20:1 to 125:1. In some embodiments, a pharmaceutical composition described herein has a molar ratio of 3E10 antibody or variant thereof, or antigen-binding fragment thereof to therapeutic polynucleotide that is of from 20:1 to 100:1. In some embodiments, a pharmaceutical composition described herein has a molar ratio of 3E10 antibody or variant thereof, or antigen-binding fragment thereof to therapeutic polynucleotide that is of from 20:1 to 75:1. In some embodiments, a pharmaceutical composition described herein has a molar ratio of 3E10 antibody or variant thereof, or antigen-binding fragment thereof to therapeutic polynucleotide that is of from 20:1 to 50:1. In some embodiments, a pharmaceutical composition described herein has a molar ratio of 3E10 antibody or variant thereof, or antigen-binding fragment thereof to therapeutic polynucleotide that is of from 20:1 to 40:1. In some embodiments, a pharmaceutical composition described herein has a molar ratio of 3E10 antibody or variant thereof, or antigen-binding fragment thereof to therapeutic polynucleotide that is of from 20:1 to 30:1. In some embodiments, a pharmaceutical composition described herein has a molar ratio of 3E10 antibody or variant thereof, or antigen-binding fragment thereof to therapeutic polynucleotide that is of from 20:1 to 25:1.

In some embodiments, a pharmaceutical composition described herein has a molar ratio of 3E10 antibody or variant thereof, or antigen-binding fragment thereof to therapeutic polynucleotide that is of from 25:1 to 200:1. In some embodiments, a pharmaceutical composition described herein has a molar ratio of 3E10 antibody or variant thereof, or antigen-binding fragment thereof to therapeutic polynucleotide that is of from 25:1 to 175:1. In some embodiments, a pharmaceutical composition described herein has a molar ratio of 3E10 antibody or variant thereof, or antigen-binding fragment thereof to therapeutic polynucleotide that is of from 25:1 to 150:1. In some embodiments, a pharmaceutical composition described herein has a molar ratio of 3E10 antibody or variant thereof, or antigen-binding fragment thereof to therapeutic polynucleotide that is of from 25:1 to 125:1. In some embodiments, a pharmaceutical composition described herein has a molar ratio of 3E10 antibody or variant thereof, or antigen-binding fragment thereof to therapeutic polynucleotide that is of from 25:1 to 100:1. In some embodiments, a pharmaceutical composition described herein has a molar ratio of 3E10 antibody or variant thereof, or antigen-binding fragment thereof to therapeutic polynucleotide that is of from 25:1 to 75:1. In some embodiments, a pharmaceutical composition described herein has a molar ratio of 3E10 antibody or variant thereof, or antigen-binding fragment thereof to therapeutic polynucleotide that is of from 25:1 to 50:1. In some embodiments, a pharmaceutical composition described herein has a molar ratio of 3E10 antibody or variant thereof, or antigen-binding fragment thereof to therapeutic polynucleotide that is of from 25:1 to 40:1. In some embodiments, a pharmaceutical composition described herein has a molar ratio of 3E10 antibody or variant thereof, or antigen-binding fragment thereof to therapeutic polynucleotide that is of from 25:1 to 30:1.

In yet other embodiments, other ranges falling with the range of about 2:1 to about 200:1 are contemplated.

In some embodiments, a pharmaceutical composition described herein has a molar ratio of 3E10 antibody or variant thereof, or antigen-binding fragment thereof to therapeutic polynucleotide that is of from about 1:1 to about 200:1. In some embodiments, a pharmaceutical composition described herein has a molar ratio of 3E10 antibody or variant thereof, or antigen-binding fragment thereof to therapeutic polynucleotide that is of from about 1:1 to about 175:1. In some embodiments, a pharmaceutical composition described herein has a molar ratio of 3E10 antibody or variant thereof, or antigen-binding fragment thereof to therapeutic polynucleotide that is of from about 1:1 to about 150:1. In some embodiments, a pharmaceutical composition described herein has a molar ratio of 3E10 antibody or variant thereof, or antigen-binding fragment thereof to therapeutic polynucleotide that is of from about 1:1 to about 125:1. In some embodiments, a pharmaceutical composition described herein has a molar ratio of 3E10 antibody or variant thereof, or antigen-binding fragment thereof to therapeutic polynucleotide that is of from about 1:1 to about 100:1. In some embodiments, a pharmaceutical composition described herein has a molar ratio of 3E10 antibody or variant thereof, or antigen-binding fragment thereof to therapeutic polynucleotide that is of from about 1:1 to about 75:1. In some embodiments, a pharmaceutical composition described herein has a molar ratio of 3E10 antibody or variant thereof, or antigen-binding fragment thereof to therapeutic polynucleotide that is of from about 1:1 to about 50:1. In some embodiments, a pharmaceutical composition described herein has a molar ratio of 3E10 antibody or variant thereof, or antigen-binding fragment thereof to therapeutic polynucleotide that is of from about 1:1 to about 30:1. In some embodiments, a pharmaceutical composition described herein has a molar ratio of 3E10 antibody or variant thereof, or antigen-binding fragment thereof to therapeutic polynucleotide that is of from about 1:1 to about 20:1. In some embodiments, a pharmaceutical composition described herein has a molar ratio of 3E10 antibody or variant thereof, or antigen-binding fragment thereof to therapeutic polynucleotide that is of from about 1:1 to about 10:1. In some embodiments, a pharmaceutical composition described herein has a molar ratio of 3E10 antibody or variant thereof, or antigen-binding fragment thereof to therapeutic polynucleotide that is of from about 1:1 to about 5:1.

In some embodiments, because 3E10 antibodies or variants thereof, or antigen-binding fragments thereof localize to tissues in vivo following systemic administration, the compositions of the present disclosure can be formulated for, and subsequently administered by, one of many common administrative routes. In some embodiments, the pharmaceutical composition is formulated for parenteral administration. In some embodiments, the parenteral administration is intramuscular administration, intravenous administration, subcutaneous administration, or intratumoral administration.

In some embodiments, the therapeutic polynucleotides of the compositions described herein are codon-optimized, e.g., to improve half-life or increase translation in a tissue in vivo. Codon-optimized refers to a polynucleotide sequence encoding a polypeptide, where at least one codon of the native polynucleotide encoding the polypeptide has been changed to improve a property of the polynucleotide sequence. In some embodiments, the improved property promotes increased transcription of mRNA coding for the polypeptide, increased stability of the mRNA (e.g., improved mRNA half-life), and/or increased translation of the polypeptide. Non-limiting examples of alterations that can be used to achieve the improved properties include changing the usage and/or distribution of codons for particular amino acids, adjusting global and/or local GC content, removing AT-rich sequences, removing repeated sequence elements, adjusting global and/or local CpG dinucleotide content, removing cryptic regulatory elements (e.g., TATA box and CCAAT box elements), removing of intron/exon splice sites, improving regulatory sequences (e.g., introduction of a Kozak consensus sequence), and removing sequence elements capable of forming secondary structure (e.g., stem-loops) in the transcribed mRNA.

### Skeletal Muscle Disorders

As reported in Example 3, 3E10 antibodies or variants thereof, or antigen-binding fragments thereof localize to skeletal muscle tissue in vivo following systemic administration. Accordingly, the compositions described herein are well suited for the delivery of therapeutic polynucleotides useful for treating disorders of skeletal muscle tissue. Accordingly, in some embodiments, the therapeutic polynucleotide includes a sequence from, or complementary to, a gene encoding a skeletal-muscle polypeptide or associated regulatory region.

Examples of proteins, and their associated genes, that are mutated in various skeletal muscle disorders are presented in Table 2. Generally, sequences encoding, or complementary to sequences encoding, any one of these proteins, and variants thereof retaining a function of the full-length protein, can be included in the therapeutic polynucleotides disclosed herein. Accordingly, in some embodiments, the skeletal-muscle polypeptide is selected from the group consisting of nebulin (NEB), skeletal muscle alpha-actin (ACTA), alpha-tropomyosin-3 (TPM3), beta-tropomyosin-2 (TPM2), troponin T1 (TNNT1), cofilin-2 (CFL2), Kelch-repeat-and-BTB-domain-containing-13 (KBTBD13), Kelch-like-family member-40 (KLHL40), Kelch-like protein 4 (KLHL4), Kelch-like-family member 41 (KLHL41), leiomodin-3 (LMOD3), myopalladin (MYPN), ryanodine receptor (RYR1), selenoprotein N (SEPN1), myotubularin (MTM1), dynamin-2 (DNM2), amphiphysin-2 (BIN1), titin (TTN), striated muscle preferentially expressed protein kinase (SPEG), slow-skeletal/beta-cardiac myosin heavy chain (MYH7) cytochrome b, cytochrome c oxidase, thymidine kinase (TK2), polymerase gamma 1 (POLG1), lysosomal enzyme acid alpha-glucosidase (GAA), glycogen-debranching enzyme (AGL), myophosphorylase (PYGM), carnitine transporter OCTN2 (SLC22A5), electron-transfer flavoprotein (ETF), ETF-dehydrogenase (ETFH), adipose triglyceride lipase (PNPLA2), skeletal muscle chloride channel (CIC1), alpha-subunit of the skeletal muscle sodium channel (SCN4A), myotonin-protein kinase (DMPK), zinc finger 9 (ZNF9), dystrophin (DMD), myotilin (MYOT), lamin A/C (LMNA), caveolin 3 (CAV3), DnaJ Heat Shock Protein Family (Hsp40) Member B6 (DNAJB6), desmin (DES), transportin 3, Heterogeneous nuclear ribonucleoprotein D-like (HNRPDL), calpain 3, dysferlin (DYSF), gamma-sarcoglycan (SGCG), alpha-sarcoglycan (SGCA), beta-sarcoglycan (SGCB), delta-sarcoglycan (SGCD), telethonin (TCAP), E3 ubiquitin-protein ligase TRIM32 (TRIM32), Fukutin-related protein (FKRP), Protein O-mannosyl-transferase 1 (POMT1), anoctamin 5 (ANO5), fukutin, Protein O-mannosyl-transferase 2 (POMT2), O-linked-mannose beta-1,2-N acetylglucosaminyltransferase (POMTnG1), dystroglycan (DAG1), plectin (PLEC1), LGMD2R, Trafficking protein particle complex subunit 11 (TRAPPC11), Mannose-1-phosphate guanyltransferase beta (GMPPB), D-ribitol-5-phosphate cytidylyltransferase (ISPD), alpha-glucosidase, LIM and senescent cell antigen-like-containing domain protein 2 (LIMS2), isoprenoid synthase domain containing (ISPD), Popeye-domain containing 1 (POPDC1), lamina-associated polypeptide 1B (TORIAIP1), Oglucosyltransferase 1 (POGLUT1), Laminin subunit alpha-2 (LAMA2), collagen alpha-1(VI) chain (COL6A1), collagen alpha-2(VI) chain (COL6A2), collagen alpha-3(VI) chain (COL6A3), double homeobox 4 (DUX4), and emerin (EMD).

Gene replacement therapy refers to a number of therapeutic techniques for delivering a functional copy of a gene to a tissue in need of the protein encoded by the gene, including DNA-based gene therapy techniques in which a functional copy of the gene is transcribed within the cell, e.g., with or without being stably integrated into the genome of the subject, gene editing therapies, such as CRISPR/Cas, that repair or replace mutant copies of the gene in the host's genome, and mRNA delivery-based approaches in which mRNA encoding the protein are delivered to the cell, eliminating the need to transcribe an exogenous copy of the gene. Researchers have developed, and continue to develop, gene replacement therapies for a diverse set of disorders, most notably genetic disorders in a subject has one or two mutant or non-functioning copies of the gene, e.g., due to mutations in the gene that cause partial or complete loss-of-function, mutations in an associated regulatory region that down-regulates gene transcription, and/or small genomic deletions.

Skeletal muscle disorders are typically characterized by abnormalities of muscle cell structure and/or metabolism, resulting in various patterns of muscle weakness and dysfunction. There are many types of genetic skeletal muscle disorders, caused by mutations in one or more of a large set of genes. Subjects with genetic skeletal muscle disorders commonly suffer from muscle weakness, motor delay, respiratory impairment, and bulbar muscle dysfunction. Because the etiology of many different forms of genetic skeletal muscle disorders have been well characterized, gene therapies offer an attractive option for treating these disorders. In fact, clinical trials for such gene therapies have been initiated for several genetic skeletal muscle disorders.

One such disorder for which a gene therapy is being developed is x-linked myotubular myopathy (XLMTM). MTM is a congenital myopathy caused by loss of function mutations in the myotubularin (MTM1) gene that affects 1 in 50,000 live male births. Pierson C R, Ann Transl Med., 3(5):61 (2015), the content of which is incorporated herein by reference. Adeno-associated virus (AAV)-mediated delivery of a gene therapy vector encoding a functional MTM1 gene has shown promise for treating MTM in mice, canine, and human subjects. See, Buj-Bello, Anna et al., Human molecular genetics, (17)14: 2132-43 (2008); Childers M K, Sci Transl Med., 6(220):220ra10 (2014); and Kaiser J., “Boys with a rare muscle disease are breathing on their own, thanks to gene therapy” doi: 10.1126/science.aax9005, the contents of which are incorporated herein by reference. Further, AAV-mediated delivery of a gene therapy vector encoding Myotubularin-related protein 2 (MTMR2), a homologue of the MTM1 gene improves motor activity and muscle strength in MTM1-deficient knock-out mice. Danièle N. et al., J Neuropathol Exp Neurol., 77(4):282-95 (2018), the content of which is incorporated herein by reference.

Similarly, gene therapy is being developed for treating Duchenne muscle dystrophy (DMD). DMD is an x-linked myopathy caused by loss-of-function mutations in the dystrophin (DMD) gene that affects 1 in 3,500-5,000 live male births. Several human clinical trials are ongoing for the treatment of DMD by AAV-mediated delivery of genes encoding smaller, functioning version of the dystrophin protein, sometimes referred to as mini-dystrophin or micro-dystrophin. Duan D., Mol Ther., 26(10):2337-56 (2018), the content of which is incorporated herein by reference. Further examples of skeletal muscle disorders for which clinical trials have been initiated include Becker muscle dystrophy and limb-girdle muscle dystrophy. Braun R. et al., Am J Phys Med Rehabil., 93(11 Suppl 3):S97-S107 (2014), the content of which is incorporated herein by reference.

Accordingly, in one aspect, the present disclosure provides methods for treating a skeletal muscle disorders in a subject by administering a composition that includes a therapeutically effective amount of a complex formed between (i) a therapeutic polynucleotide having a 3E10 or 3E10 variant binding domain and an effector domain that includes a sequence from, or complementary to, a gene encoding a skeletal muscle polypeptide or associated regulatory region, and (ii) a 3E10 antibody or variant thereof, or antigen-binding fragment thereof.

Although, in some embodiments, a skeletal muscle polypeptide encoded by a therapeutic polynucleotide (e.g., a DNA or mRNA molecule) is a wild-type version of the skeletal muscle protein, it will be appreciated that naturally occurring variants or synthetically engineered versions of a skeletal muscle protein may also find use in the compositions and methods described herein.

In some embodiments, the effector domain includes a sequence from, or complementary to, a gene encoding a skeletal muscle protein. Non-limiting examples of skeletal muscle proteins include nebulin (NEB), skeletal muscle alpha-actin (ACTA), alpha-tropomyosin-3 (TPM3), beta-tropomyosin-2 (TPM2), troponin T1 (TNNT1), cofilin-2 (CFL2), Kelch-repeat-and-BTB-domain-containing-13 (KBTBD13), Kelch-like-family member-40 (KLHL40), Kelch-like protein 4 (KLHL4), Kelch-like-family member 41 (KLHL41), leiomodin-3 (LMOD3), myopalladin (MYPN), ryanodine receptor (RYR1), selenoprotein N (SEPN1), myotubularin (MTM1), dynamin-2 (DNM2), amphiphysin-2 (BIN1), titin (TTN), striated muscle preferentially expressed protein kinase (SPEG), slow-skeletal/beta-cardiac myosin heavy chain (MYH7) cytochrome b, cytochrome c oxidase, thymidine kinase (TK2), polymerase gamma 1 (POLG1), lysosomal enzyme acid alpha-glucosidase (GAA), glycogen-debranching enzyme (AGL), myophosphorylase (PYGM), carnitine transporter OCTN2 (SLC22A5), electron-transfer flavoprotein (ETF), ETF-dehydrogenase (ETFH), adipose triglyceride lipase (PNPLA2), skeletal muscle chloride channel (CIC1), alpha-subunit of the skeletal muscle sodium channel (SCN4A), myotonin-protein kinase (DMPK), zinc finger 9 (ZNF9), dystrophin (DMD), myotilin (MYOT), lamin A/C (LMNA), caveolin 3 (CAV3), DnaJ Heat Shock Protein Family (Hsp40) Member B6 (DNAJB6), desmin (DES), transportin 3 (TNPO3), Heterogeneous nuclear ribonucleoprotein D-like (HNRPDL), calpain 3, dysferlin (DYSF), gamma-sarcoglycan (SGCG), alpha-sarcoglycan (SGCA), beta-sarcoglycan (SGCB), delta-sarcoglycan (SGCD), telethonin (TCAP), E3 ubiquitin-protein ligase TRIM32 (TRIM32), Fukutin-related protein (FKRP), Protein O-mannosyl-transferase 1 (POMT1), anoctamin 5 (ANO5), fukutin, Protein O-mannosyl-transferase 2 (POMT2), O-linked-mannose beta-1,2-N acetylglucosaminyltransferase (POMTnG1), dystroglycan (DAG1), plectin (PLEC1), LGMD2R, Trafficking protein particle complex subunit 11 (TRAPPC11), Mannose-1-phosphate guanyltransferase beta (GMPPB), D-ribitol-5-phosphate cytidylyltransferase (ISPD), alpha-glucosidase, LIM and senescent cell antigen-like-containing domain protein 2 (LIMS2), isoprenoid synthase domain containing (ISPD), Popeye-domain containing 1 (POPDC1), lamina-associated polypeptide 1B (TORIAIP1), O-glucosyltransferase 1 (POGLUT1), Laminin subunit alpha-2 (LAMA2), collagen alpha-1(VI) chain (COL6A1), collagen alpha-2(VI) chain (COL6A2), collagen alpha-3(VI) chain (COL6A3), double homeobox 4 (DUX4), and emerin (EMD).

In some embodiments, the subject has a skeletal muscle disorder. For example, in some embodiments, the subject carries a skeletal muscle gene having a partial or complete loss-of-function mutation. Accordingly, in some embodiments, the therapeutic polynucleotide administered to the subject encodes for a functional copy of a polypeptide corresponding to the mutated gene in the subject. However, in some instances, such as with MTMR2-mediated gene therapy for x-linked myotubular myopathy (described above), the therapeutic polypeptide encodes for a homologue of the protein encoded by the mutant gene in the subject, a protein that has partially redundant function, and/or a protein that functions in a partially-redundant pathway as the protein encoded by the mutant gene in the subject.

In some embodiments, the skeletal muscle disorder is a non-dystrophic genetic myopathy. Non-limiting examples of non-dystrophic genetic myopathies include nemaline myopathy, core myopathy (central and multi-minicore), centronuclear myopathy/myotubular myopathy (XLMTM), congenital fiber-type disproportion myopathy, myosin storage myopathy, mitochondrial myopathy, genetic myopathy, Metabolic myopathy (lipid storage disease), congenital myotonia, and paramyotonia congenital. For a review of example non-dystrophic genetic myopathies see, for example, Muscle Cell and Tissue-Current Status of Research Field, Edited by Kunihiro Sakuma, Chapter 6 “Genetic Myopathies” (2018), the content of which is incorporated herein by reference.

In some embodiments, the skeletal muscle disorder is a dystrophic genetic myopathy. Non-limiting examples of dystrophic genetic myopathies include a myotonic dystrophy (DM1/DM2), Duchenne muscle dystrophy, Becker muscle dystrophy, autosomal-dominant form of limb-girdle muscle dystrophy (LGMD1), autosomal-recessive form of limb-girdle muscle dystrophy (LGMD2), congenital muscle dystrophy, facioscapulohumeral musular dystrophy, and Emery-dreifuss muscle dystrophy. For a review of example dystrophic genetic myopathies see, for example, Muscle Cell and Tissue-Current Status of Research Field, Edited by Kunihiro Sakuma, Chapter 6 “Genetic Myopathies” (2018), the content of which is incorporated herein by reference.

Each of the classes of skeletal muscle disorders listed above has been associated with a mutation in one or more skeletal muscle genes. Examples of the genes found to be associated with particular skeletal muscle disorders are listed in Table 2, below. Accordingly, in some embodiments, a subject with a particular skeletal muscle disorder is treated by administration of a 3E10-therapeutic polynucleotide complex where the therapeutic polynucleotide encodes for, or includes a sequence complementary to a gene encoding, a polypeptide corresponding to an associated gene in Table 2. For example, in one embodiment, a therapeutic polynucleotide encoding a polypeptide associated with the myotubularin (MTM1) protein is used for the treatment of a type of myotubular myopathy, e.g., x-linked myotubular myopathy (XLMTM).

TABLE 2
Example genes found to be mutated in various skeletal muscle disorders.

| Skeletal muscle disorder | Mutant Protein (Genes) |
|---|---|
| Nemalin myopathy | Nebulin (NEB = NG_009382.2); Homo sapiens |
| | nebulin (NEB), RefSeqGene (LRG_202) on |
| | chromosome 2 |
| | Skeletal muscle alpha-actin (ACTA1 = |
| | NG_006672.1); Homo sapiens actin |
| | alpha 1, skeletal muscle (ACTA1), RefSeqGene |
| | (LRG_429) on chromosome 1 |
| | Alpha-tropomyosin-3 (TPM3 = NG_008621.1); |
| | Homo sapiens tropomyosin 3 (TPM3), |
| | RefSeqGene (LRG_681) on chromosome 1 |
| | Beta-tropomyosin-2 (TPM2 = NG_011620.1); |
| | Homo sapiens tropomyosin 2 (TPM2), |
| | RefSeqGene (LRG_680) on chromosome 9 |
| | Troponin T1 (TNNT1 = NG_011829.2); |
| | Homo sapiens troponin T1, slow skeletal |
| | type (TNNT1), RefSeqGene (LRG_679) on |
| | chromosome 19 |
| | Cofilin-2 (CFL2 = NG_012740.1); Homo |
| | sapiens cofilin 2 (CFL2), RefSeqGene |
| | (LRG_213) on chromosome 14 |
| | Kelch-repeat-and-BTB-domain-containing-13 |
| | (KBTBD13 = NG_021411.1); Homo sapiens |
| | kelch repeat and BTB domain containing |
| | 13 (KBTBD13), RefSeqGene (LRG_682) on |
| | chromosome 15 |
| | Kelch-like-family member-40 (KLHL40 = |
| | NG_033035.1); Homo sapiens kelch like |
| | family member 40 (KLHL40), RefSeqGene |
| | on chromosome 3 |
| | Kelch-like protein 4 (KLHL4 = NG_012815.1); |
| | Homo sapiens kelch like family member 4 |
| | (KLHL4), RefSeqGene on chromosome X |
| | Kelch-like-family member 41 (KLHL41 = |
| | NG_042051.1); Homo sapiens kelch like |
| | family member 41 (KLHL41), RefSeqGene |
| | on chromosome 2 |
| | Leiomodin-3 (LMOD3 = NG_041828.1); |
| | Homo sapiens leiomodin 3 (LMOD3), |
| | RefSeqGene on chromosome 3 |
| | Myopalladin (MYPN = NG_032118.1); |
| | Homo sapiens myopalladin (MYPN), |
| | RefSeqGene (LRG_410) on chromosome 10 |
| Core myopathy | Ryanodine receptor channel (RYR1 = |
| (Central and | NG_008866.1); Homo sapiens ryanodine |
| multiminicore) | receptor 1 (RYR1), RefSeqGene (LRG_766) |
| | on chromosome 19 |
| | Selenoprotein N (SELENON = NG_009930.1); |
| | Homo sapiens selenoprotein N (SELENON), |
| | RefSeqGene (LRG_857) on chromosome 1 |
| Centronuclear | Myotubularin (MTM1 = NG_008199.1); |
| myopathy/Myotubular | Homo sapiens myotubularin 1 (MTM1), |
| myopathy (XLMTM) | RefSeqGene (LRG_839) on chromosome X |
| | Synamin-2 (DNM2 = NG_032118.1); Homo |
| | sapiens myopalladin (MYPN), RefSeqGene |
| | (LRG_410) on chromosome 10 |
| | Amphiphysin-2 (BIN1 = NG_012042.1); |
| | Homo sapiens bridging integrator 1 (BIN1), |
| | RefSeqGene (LRG_873) on chromosome 2 |
| | Ryanodine receptor channel (RYR1 = |
| | NG_008866.1); Homo sapiens ryanodine |
| | receptor 1 (RYR1), RefSeqGene (LRG_766) |
| | on chromosome 19 |
| | titin (TTN = NG_011618.3); Homo sapiens |
| | titin (TTN), RefSeqGene (LRG_391) on |
| | chromosome 2 |
| | Striated muscle preferentially expressed |
| | protein kinase (SPEG = NG_051022.1); Homo |
| | sapiens striated muscle enriched protein |
| | kinase (SPEG), RefSeqGene on chromosome 2 |
| Congenital | Skeletal muscle alpha-actin (ACTA1 = |
| fiber-type | NG_006672.1); Homo sapiens actin alpha 1, |
| disproportion | skeletal muscle (ACTA1), RefSeqGene |
| myopathy | (LRG_429) on chromosome 1 |
| | Alpha-tropomyosin-3 (TPM3 = NG_008621.1); |
| | Homo sapiens tropomyosin 3 (TPM3), |
| | RefSeqGene (LRG_681) on chromosome 1 |
| | Ryanodine receptor channel (RYR1 = |
| | NG_008866.1); Homo sapiens ryanodine |
| | receptor 1 (RYR1), RefSeqGene (LRG 766) |
| | on chromosome 19 |
| Myosin storage | Slow-skeletal/beta-cardiac myosin heavy |
| myopathy | chain (MYH7 = NG_007884.1); Homo sapiens |
| | myosin heavy chain 7 (MYH7), RefSeqGene |
| | (LRG 384) on chromosome 14 |
| Mitochondrial | Cytochrome b, cytochrome c oxidase, |
| myopathy | thymidine kinase (TK2 = NG_016862.1); |
| | Homo sapiens thymidine kinase 2 (TK2), |
| | RefSeqGene on chromosome 16; nuclear |
| | gene for mitochondrial product |
| | Polymerase gamma 1 (POLG = NG_008218.2); |
| | Homo sapiens DNA polymerase gamma, |
| | catalytic subunit (POLG), RefSeqGene |
| | (LRG_765) on chromosome 15 |
| Metabolic | Lysosomal enzyme acid alpha-glucosidase |
| myopathy | (GAA = NG_009822.1); Homo sapiens alpha |
| (glycogen | glucosidase (GAA), RefSeqGene (LRG_673) |
| storage disease) | on chromosome 17 |
| | Glycogen-debranching enzyme (AGL = |
| | NG_012865.1); Homo sapiens amylo-alpha- |
| | 1,6-glucosidase, 4-alpha-glucanotransferase |
| | (AGL), RefSeqGene on chromosome 1 |
| | Myophosphorylase (PYGM = NG_013018.1); |
| | Homo sapiens glycogen phosphorylase, |
| | muscle associated (PYGM), RefSeqGene on |
| | chromosome 11 |
| Metabolic | Carnitine transporter OCTN2 (SLC22A5 = |
| myopathy | NG_008982.2); Homo sapiens solute carrier |
| (lipid storage | family 22 member 5 (SLC22A5), RefSeqGene |
| disease) | on chromosome 5 |
| | Electron-transfer flavoprotein (ETF) |
| | ETF-dehydrogenase (ETFH) |
| | Adipose triglyceride lipase (PNPLA2 = |
| | NG_023394.1); Homo sapiens patatin like |
| | phospholipase domain containing 2 (PNPLA2), |
| | RefSeqGene on chromosome 11 |
| Congenital | Skeletal muscle chloride channel (CLCN1 = |
| myotonia | NG_009815.2); Homo sapiens chloride voltage- |
| | gated channel 1 (CLCN1), RefSeqGene on |
| | chromosome 7 |
| Paramyotonia | Alpha-subunit of the skeletal muscle sodium |
| congenita | channel (SCN4A = NG_011699.1); Homo sapiens |
| | sodium voltage-gated channel alpha subunit |
| | 4 (SCN4A), RefSeqGene on chromosome 17 |
| Myotonic | Myotonin-protein kinase (DMPK = NG_009784.1); |
| dystrophy | Homo sapiens DM1 protein kinase (DMPK), |
| (DM1/DM2) | RefSeqGene on chromosome 19 |
| | Zinc finger 9 (CNBP = NG_011902.1); Homo |
| | sapiens CCHC-type zinc finger nucleic acid |
| | binding protein (CNBP), RefSeqGene on |
| | chromosome 3 |
| Duchenne and | Dystrophin (DMD = NG_012232.1; Homo sapiens |
| Becker muscle | dystrophin (DMD), RefSeqGene (LRG_199) on |
| dystrophy | chromosome X) |
| Autosomal-dominant | Myotilin (MYOT = NG_008894.1); Homo sapiens |
| form of limb- | myotilin (MYOT), RefSeqGene (LRG_201) on |
| girdle muscle | chromosome 5 |
| dystrophy (LGMD1) | Lamin A/C (LMNA = NG_ 008692.2); Homo sapiens |
| | lamin A/C (LMNA), RefSeqGene (LRG_254) on |
| | chromosome 1 |
| | Caveolin 3 (CAV3 = NG_008797.2); Homo sapiens |
| | caveolin 3 (CAV3), RefSeqGene (LRG_329) on |
| | chromosome 3 |
| | DnaJ Heat Shock Protein Family (Hsp40) |
| | Member B6 (DNAJB6 = NG_032573.1); Homo sapiens |
| | DnaJ heat shock protein family (Hsp40) member |
| | B6 (DNAJB6), RefSeqGene on chromosome 7 |
| | Desmin (DES = NG_008043.1); Homo sapiens |
| | desmin (DES), RefSeqGene (LRG_380) on |
| | chromosome 2 |
| | Transportin 3 (TNPO3 = NG_023428.1); Homo |
| | sapiens transportin 3 (TNPO3), RefSeqGene |
| | on chromosome 7 Heterogeneous nuclear |
| | ribonucleoprotein D-like (HNRNPDL = |
| | NG_029681.1); Homo sapiens heterogeneous |
| | nuclear ribonucleoprotein D like (HNRNPDL), |
| | RefSeqGene on chromosome 4 |
| Autosomal- | Calpain 3 (CAPN3 = NG_008660.1); Homo sapiens |
| recessive form | calpain 3 (CAPN3), RefSeqGene (LRG_849) on |
| of limb-girdle | chromosome 15 |
| muscle dystrophy | Dysferlin (DYSF = NG_008694.1); Homo sapiens |
| (LGMD2) | dysferlin (DYSF), RefSeqGene (LRG_845) on |
| | chromosome 2 |
| | Gamma-sarcoglycan (SGCG = NG_008759.1); |
| | Homo sapiens sarcoglycan gamma (SGCG), |
| | RefSeqGene (LRG_207) on chromosome 13 |
| | Alpha-sarcoglycan (SGCA = NG_008889.1); |
| | Homo sapiens sarcoglycan alpha (SGCA), |
| | RefSeqGene (LRG_203) on chromosome 17 |
| | Beta-sarcoglycan (SGCB = NG_008891.1); |
| | Homo sapiens sarcoglycan beta (SGCB), |
| | RefSeqGene (LRG_204) on chromosome 4 |
| | Delta-sarcoglycan (SGCD = NG_008693.2); |
| | Homo sapiens sarcoglycan delta (SGCD), |
| | RefSeqGene (LRG_205) on chromosome 5 |
| | Telethonin (TCAP = NG_008892.1); |
| | Homo sapiens titin-cap (TCAP), RefSeqGene |
| | (LRG_210) on chromosome 17 |
| | E3 ubiquitin-protein ligase TRIM32 |
| | (TRIM32 = NG_011619.1); Homo sapiens |
| | tripartite motif containing 32 (TRIM32), |
| | RefSeqGene (LRG_211) on chromosome 9 |
| | Fukutin-related protein (FKRP = |
| | NG_008898.2); Homo sapiens fukutin |
| | related protein (FKRP), RefSeqGene |
| | (LRG_761) on chromosome 19 |
| | Protein O-mannosyl-transferase 1 |
| | (POMT1 = LC030233.1); Homo sapiens |
| | POMT1 mRNA, complete cds, contains |
| | 9-bp deletion |
| | Fukutin (FKTN = NG_008754.1); Homo sapiens |
| | fukutin (FKTN), RefSeqGene (LRG_434) |
| | on chromosome 9 |
| | Protein O-mannosyl-transferase 2 (POMT2 = |
| | NG_008897.1); Homo sapiens protein O- |
| | mannosyltransferase 2 (POMT2), RefSeqGene |
| | (LRG_844) on chromosome 14 |
| | O-linked-mannose beta-1,2-N |
| | acetylglucosaminyltransferase (POMGNT1 = |
| | NG_009205.3; Homo sapiens protein O-linked |
| | mannose N-acetylglucosaminyltransferase 1 |
| | (beta 1,2-) (POMGNT1), RefSeqGene (LRG_701) |
| | on chromosome 1) |
| | dystroglycan (DAG1 = NG_013230.4); Homo sapiens |
| | dystroglycan 1 (DAG1), RefSeqGene (LRG_854) |
| | on chromosome 3 |
| | Titin (TTN = NG_011618.3); Homo sapiens |
| | titin (TTN), RefSeqGene (LRG_391) on |
| | chromosome 2 |
| | Anoctamin 5 (ANO5 = NG_015844.1); Homo sapiens |
| | anoctamin 5 (ANO5), RefSeqGene (LRG_868) on |
| | chromosome 11 |
| | Plectin (PLEC = NG_ 012492.1); Homo sapiens |
| | plectin (PLEC), RefSeqGene on chromosome 8 |
| | Desmin (DES = NG_008043.1); Homo sapiens |
| | desmin (DES), RefSeqGene (LRG_380) on |
| | chromosome 2 |
| | Trafficking protein particle complex |
| | subunit 11 (TRAPPC11 = NG_033102.1); Homo |
| | sapiens trafficking protein particle |
| | complex subunit 11 (TRAPPC11), RefSeqGene |
| | on chromosome 4 |
| | Mannose-1-phosphate guanyltransferase |
| | beta (GMPPB = NG_033731.2); Homo sapiens |
| | GDP-mannose pyrophosphorylase B (GMPPB), |
| | RefSeqGene on chromosome 3 |
| | D-ribitol-5-phosphate cytidylyltransferase |
| | (ISPD) lysosomal enzyme acid alpha- |
| | glucosidase (GAA = NG_009822.1); Homo sapiens |
| | alpha glucosidase (GAA), RefSeqGene |
| | (LRG_673) on chromosome 17 |
| | LIM and senescent cell antigen-like- |
| | containing domain protein 2 (LIMS2 = |
| | NG_042235.1); Homo sapiens LIM zinc |
| | finger domain containing 2 (LIMS2), |
| | RefSeqGene on chromosome 2 |
| | Popeye-domain containing 1 (BVES = |
| | NG_046732.1); Homo sapiens blood vessel |
| | epicardial substance (BVES), RefSeqGene |
| | on chromosome 6 |
| | Lamina-associated polypeptide 1B |
| | (TOR1AIP1 = NG_042316.1); Homo sapiens |
| | torsin 1A interacting protein 1 (TOR1AIP1), |
| | RefSeqGene on chromosome 1 |
| | O-glucosyltransferase 1 (POGLUT1 = |
| | NG_034115.1); Homo sapiens protein O- |
| | glucosyltransferase 1 (POGLUT1), |
| | RefSeqGene on chromosome 3 |
| Congenital | Laminin subunit alpha-2 (LAMA2 = NG_008678.1); |
| muscle | Homo sapiens laminin subunit alpha 2 (LAMA2), |
| dystrophies | RefSeqGene (LRG_409) on chromosome 6 |
| | Collagen alpha-1(VI) chain (COL6A1 = |
| | NG_008674.1); Homo sapiens collagen type VI |
| | alpha 1 chain (COL6A1), RefSeqGene (LRG_475) |
| | on chromosome 21 |
| | Collagen alpha-2(VI) chain (COL6A2 = |
| | NG_008675.1); Homo sapiens collagen type |
| | VI alpha 2 chain (COL6A2), RefSeqGene |
| | (LRG_476) on chromosome 21 |
| | Collagen alpha-3(VI) chain (COL6A3 = |
| | NG_008676.1); Homo sapiens collagen type |
| | VI alpha 3 chain (COL6A3), RefSeqGene |
| | (LRG_473) on chromosome 2 |
| | Protein O-mannosyl-transferase 1 (POMT1 = |
| | NG_008896.2); Homo sapiens protein O- |
| | mannosyltransferase 1 (POMT1), RefSeqGene |
| | (LRG_842) on chromosome 9 |
| | Protein O-mannosyl-transferase 2 (POMT2 = |
| | NG_008897.1); Homo sapiens protein O- |
| | mannosyltransferase 2 (POMT2), RefSeqGene |
| | (LRG_844) on chromosome 14 |
| Facioscapulohumeral | Double homeobox 4 (DUX4 = NG_034189.3); |
| musular dystrophy | Homo sapiens double homeobox 4 (DUX4), |
| | RefSeqGene (LRG_1075) on chromosome 4 |
| Emery-dreifuss | Emerin (EMD = NG_008677.1); Homo sapiens |
| muscle dystrophy | emerin (EMD), RefSeqGene (LRG_745) on |
| | chromosome X |
| | Lamin A/C (LMNA = NG_008692.2); Homo sapiens |
| | lamin A/C (LMNA), RefSeqGene (LRG 254) on |
| | chromosome 1 |

### EXAMPLES

With respect to the experiments below, standard 3E10 sequence was used except wherein noted to be the D3IN variant (e.g., Example 4). Both standard 3E10 and the D3IN variant were used as full length antibodies.

### Example 1—Carrier DNA Enhances mRNA to Non-Tumor Tissue

2 μg of fluorescently labeled mRNA was mixed with 20 μg of 3E10-D31N with or without carrier DNA (5 μg) for 15 minutes at room temperature. mRNA complexed to 3E10 was injected to fetuses at E15.5. 24-48 hours after treatment, fetuses were harvested and analyzed for mRNA delivery using IVIS imaging.

Without carrier DNA, 3E10-D31N complexed to mRNA was rapidly cleared from fetuses at 24 hours. The addition of carrier DNA, however, resulted in detectable mRNA signal in multiple tissues of the fetus at 48 hours.

### Example 2-3E10 (D31N) Complexed with mRNA and Carrier DNA Results in Sustained Levels Protein Expression

10 μg of luciferase mRNA and 10 μg of single stranded carrier DNA (60 nts) was mixed with 100 μg of 3E10 (WT) or 3E10 (D31N) for 15 minutes at room temperature. mRNA complexed to 3E10 was injected intramuscularly (IM) in the right quadricep of each mouse. Luciferase expression was monitored over 6 days.

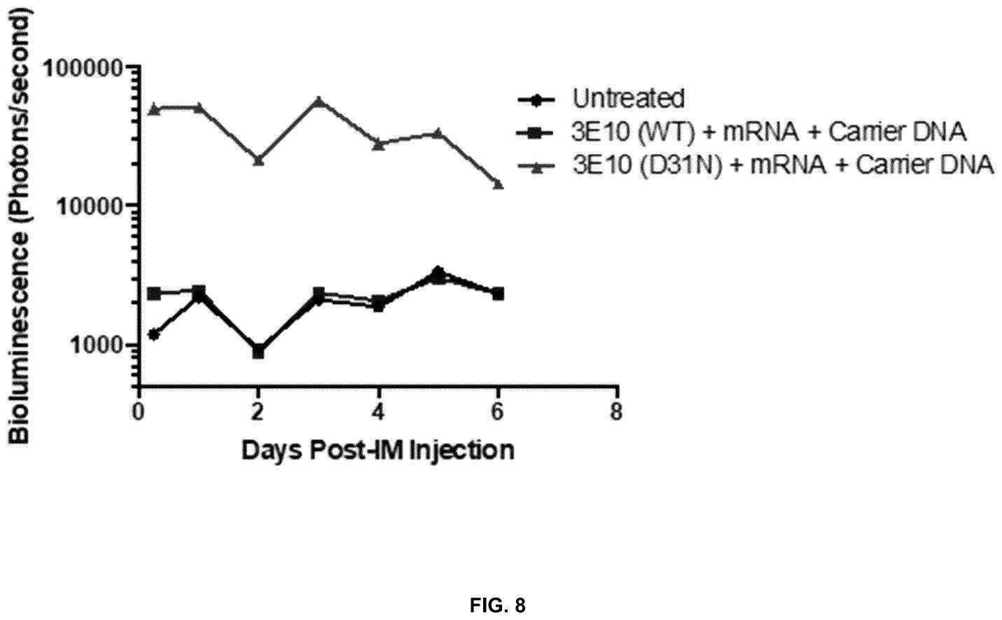

As seen in FIG. 8, administration of 3E10 (D31N) complexed with mRNA and carrier DNA resulted in sustained levels of luciferase expression, while 3E10 (WT) complexed to mRNA and carrier DNA failed to produce any appreciable signal above background.

### Example 3—Distribution of IV Injected 3E10 In Vivo

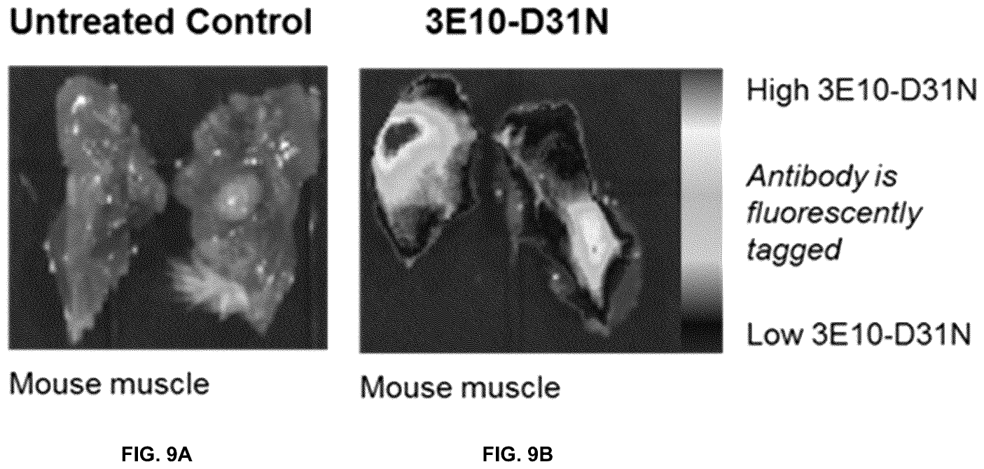

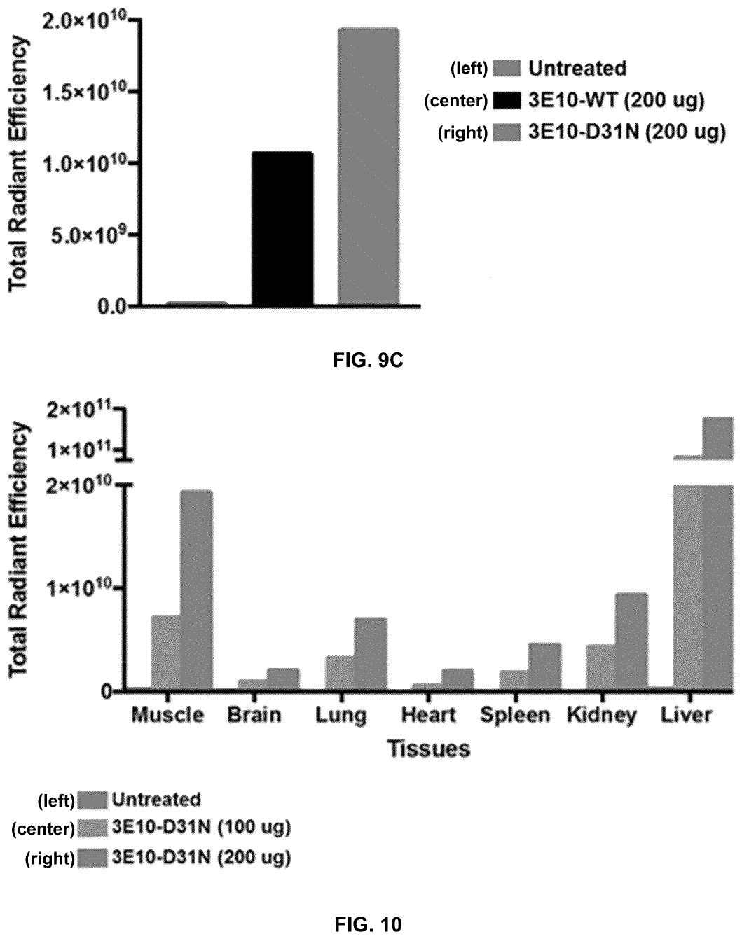

Distribution of IV injected 3E10 to muscle was investigated. Mice were injected intravenously with 200 μg of 3E10, WT or D31N, labeled with VivoTag680 (Perkin Elmer). Four hours after injection, muscle was harvested and imaged by IVIS (Perkin Elmer) (FIGS. 9A and 9B). Quantification of IVIS image demonstrates that 3E10-D31N achieves higher distribution to muscle when compared to 3E10-WT (FIG. 9C).

Dose-dependent biodistribution of 3E10-D31N to tissues was investigated. Mice were injected intravenously with 100 μg or 200 μg of 3E10-D31N labeled with VivoTag680 (Perkin Elmer). 24 hours after injection, tissues were harvested and imaged by IVIS (Perkin Elmer). Quantification of tissue distribution demonstrated a dose-dependent, two-fold increase in muscle accumulation without a commensurate increase in multiple tissues including liver (FIG. 10)

### Example 4—Molecular Modeling of 3E10 and Engineered Variants Thereof

WT HEAVY CHAIN scFv SEQUENCE
(SEQ ID NO: 108)
E VQL VESGGGL VKPGGSRKLS CAASGFTFSD
YGMHWVRQAP EKGLEWVAYI SSGSSTIYYA
DTVKGRFTIS RDNAKNTLFL QMTSLRSEDT
AMYYCARRGL LLDYWGQGTT LTVS
LIGHT CHAIN scFv SEQUENCE
(SEQ ID NO: 109)
D IVLTQSPASL AVSLGQRATI SCRASKSVST SSYSYMHWYQ
QKPGQPPKLL IKYASYLESG VPARFSGSGS GTDFTLNIHP
VEEEDAATYY CQHSREFPWT FGGGTKLEIK RADAAPGGGG
SGGGGSGGGGS

Molecular modeling of 3E10 (Pymol) revealed a putative Nucleic Acid Binding pocket (NAB1) (FIGS. 11A-11B). Mutation of aspartic acid at residue 31 of CDR1 to asparagine increased the cationic charge of this residue and enhanced nucleic acid binding and delivery in vivo (3E10-D31N).

Mutation of aspartic acid at residue 31 of CDR1 to arginine (3E10-D31R), further expanded the cationic charge while mutation to lysine (3E10-D31K) changed charge orientation (FIG. 14A).

NAB1 amino acids predicted from molecular modeling have been underlined in the heavy and light chain sequences above. FIG. 11B is an illustration showing molecular modeling of 3E10-scFv (Pymol) with NAB1 amino acid residues illustrated with punctate dots.

### Example 5—Intermuscular Injection of 3E10 (D31N) Complexed with mRNA Results in Sustained Protein Expression in Skeletal Muscle

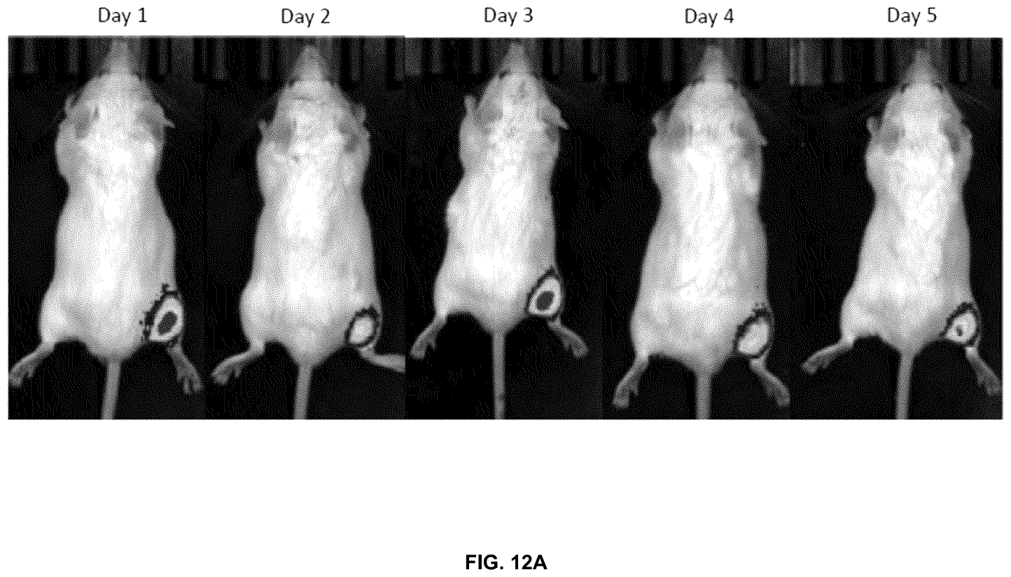

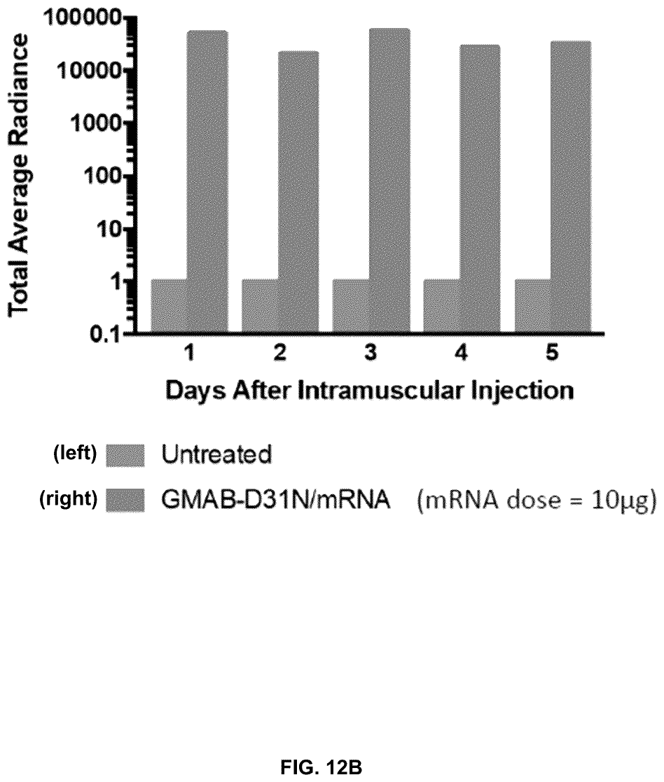

It was next investigated whether intramuscular administration of a 3E10 (D31N)-mRNA complex would result in sustained expression of the mRNA in skeletal muscle. Briefly, complexes of 3E10 (D31N) and mRNA encoding green fluorescent protein, a luciferase, having the sequence GFP_mRNA shown below as (SEQ ID NO: 110), were formed by mixing 3E10 (D31N) and mRNA at a 20:1 molar ratio. The resulting complex was administered by intermuscular injection into the hind-leg skeletal muscle of a mouse. Bioluminescence in the skeletal muscle, indicating expression of the luciferase from the injected mRNA, was imaged (FIG. 12A) and quantified (FIG. 12B) over five days. As shown in FIG. 12B, expression levels of the mRNA-encoded luciferase were sustained for at least five days.

### Example 6-3E10 (D31N) Protects mRNA Against RNA Degradation

It was next investigated whether complexing mRNA with 3E10 (D31N) would protect the mRNA from degradation. Briefly, complexes of 3E10 (D31N) and mRNA encoding green fluorescent protein, a luciferase, having the sequence GFP_mRNA shown below as (SEQ ID NO:110), were formed by mixing 3E10 (D31N) and mRNA at a 20:1 molar ratio. The free mRNA and the 3E10-mRNA complex were then incubated with 1% serum, 10% serum, or 16 μg/mL RNAse A. Gel electrophoresis analysis of the reactions was performed (FIG. 13A). As shown in FIG. 13A, free mRNA was degraded by incubation with each of 1% serum, 10% serum, and RNAse A. However, no apparent RNA degradation was observed when the complexed mRNA was incubated with any of 1% serum, 10% serum, or RNAse A, suggesting that 3E10 (D31N) protects mRNA from degradation.

>GFP_mRNA
(SEQ ID NO: 110)
AUGGUGAGCAAGGGCGAGGAGCUGUUCACCGGGGUGGUGCCCAUCC
UGGUCGAGCUGGACGGCGACGUAAACGGCCACAAGUUCAGCGUGU
CCGGCGAGGGCGAGGGCGAUGCCACCUACGGCAAGCUGACCCUGA
AGUUCAUCUGCACCACCGGCAAGCUGCCCGUGCCCUGGCCCACCC
UCGUGACCACCCUGACCUACGGCGUGCAGUGCUUCAGCCGCUACC
CCGACCACAUGAAGCAGCACGACUUCUUCAAGUCCGCCAUGCCCG
AAGGCUACGUCCAGGAGCGCACCAUCUUCUUCAAGGACGACGGCA
ACUACAAGACCCGCGCCGAGGUGAAGUUCGAGGGCGACACCCUGG
UGAACCGCAUCGAGCUGAAGGGCAUCGACUUCAAGGAGGACGGCA
ACAUCCUGGGGCACAAGCUGGAGUACAACUACAACAGCCACAACG
UCUAUAUCAUGGCCGACAAGCAGAAGAACGGCAUCAAGGUGAACU
UCAAGAUCCGCCACAACAUCGAGGACGGCAGCGUGCAGCUCGCCG
ACCACUACCAGCAGAACACCCCCAUCGGCGACGGCCCCGUGCUGC
UGCCCGACAACCACUACCUGAGCACCCAGUCCGCCCUGAGCAAAG
ACCCCAACGAGAAGCGCGAUCACAUGGUCCUGCUGGAGUUCGUGA
CCGCCGCCGGGAUCACUCUCGGCAUGGACGAGCUGUACAAGUAA

Next, it was investigated whether mRNA complexed at lower molar ratios were also protected against RNA degradation. Briefly, complexes of 3E10 (D31N) and mRNA encoding green fluorescent protein (GFP_mRNA; SEQ ID NO:110) were formed by mixing 3E10 (D3IN) and mRNA at a 2:1 molar ratio. The free mRNA and the 3E10-mRNA complex were then incubated with RNAse A under the conditions described above. Gel electrophoresis analysis of the reactions was performed (FIG. 13B). As shown in FIG. 13B, free mRNA was completely degraded by incubation with RNAse A. However, complexing the mRNA with 3E10 (D31N) at a 2:1 molar ratio resulted in some protection of the mRNA against degradation, as indicated by the presence of an RNA signal in the well, indicating the presence of intact 3E10 (D31N)-mRNA complex. The protection provided at a 2:1 molar ratio appears to be less than the protection afforded the mRNA when complexed at a 20:1 molar ratio.

### Example 7-3E10 (D31N) Binds Poly-deoxyribonucleotides with Greater Affinity than 3E10

The binding affinities of full-length 3E10 and 3E10 (D31N) antibodies for various single-stranded poly-deoxyribonucleotides was investigated. Briefly, 10 μM 5′ biotinylated nucleic acids were diluted 1:10,000 in ELISA buffer, for a final concentration of 0.001 μM. 100 μL of diluted nucleic acid was added to streptavidin pre-coated 96-well plates (Thermo Scientific #15502) and incubated for 30 minutes at RT. While incubating, serial dilutions of 3E10 were prepared in ELISA buffer. The plate was washed 3× with 1× TBST. 100 μL of 3E10 diluted in ELISA buffer was added and the mixture was incubated for 4 hours at 4° C. with rocking. The plate was washed 3× with 1× TBST. Anti-Fc HRP Ab was diluted according to manufacturer's specifications in 1× TBST. Goat anti-human antibody from Invitrogen was diluted 1:20,000. 100 μL of Ab was added to the plate and incubated at RT for 1.5 hours with rocking. The plate was washed 3× with 1× TBST. 100 μL of ECL Pico was added to wells and developed for 5 minutes in the dark. The plate luminescence was then read on a plate reader.

As shown in FIGS. 14A-14D, 3E10 (D31N) binds to each species of poly-deoxyribonucleotides with significantly stronger affinity than does 3E10. The experiment also revealed an apparent preference for 3E10 (D3IN) binding to poly-dT (FIG. 14D), relative to the other three poly-deoxyribonucleotides (FIGS. 14A-14C). Further, both 3E10 (D31N) and 3E10 appear to bind poly-dA (FIG. 14C) with lower affinity than the other three poly-deoxyribonucleotides (FIGS. 14A-14B and 14D).

### Example 8-3E10 (D31N) Binds Poly-ribonucleotides with Greater Affinity than 3E10

The binding affinities of 3E10 and 3E10 (D3IN) for various single-stranded poly-deoxyribonucleotides and poly-ribonucleotides was investigated, as described in Example 7. As shown in FIGS. 15A-15J, 3E10 (D3IN) binds to each species of poly-deoxyribonucleotides and each species of poly-ribonucleotides with significantly stronger affinity than does 3E10. While 3E10 tended to bind the poly-deoxyribonucleotide and poly-ribonucleotide version of each nucleotide base with similar affinity—that is, 3E10 bound poly-dG with similar affinity as poly-rG (FIG. 15A), poly-dC with similar affinity as poly-rC (FIG. 15C), and poly-dA with similar affinity as poly-rA (FIG. 15E)-3E10 (D31N) had a clear preference for binding poly-rC over poly-dC (FIG. 15D), and for binding poly-dA over poly-rA (FIG. 15F). When the results of the 3E10 (D31N) poly-ribonucleotide binding assays were considered together, 3E10 (D31N) showed strongest binding to poly-rl and poly-rG, and the weakest binding to poly-rA (FIG. 15K).

### Example 9-3E10-Mediates Delivery of RIG-I Ligand, and Stimulation of RIG-I Activity

RIG-I reporter cells (HEK-Lucia RIG-I, Invivogen) were seeded at 50,000 cells per well and treated with RIG-I ligands (1 μg) or ligands complexed to 3E10-D3IN (20 μg).

This assay uses a cell line with a luciferase reporter that is activated when there is induction of interferons. RIG-I ligands were purchased from Invivogen. Below are brief descriptions of each.

3p-hpRNA (89 mer, hairpin RNA)
(SEQ ID NO: 111)
5′pppGGAGCAAAAGCAGGGUGACAAAGACAUAAUGG
AUCCAAACACUGUGUCAAGCUUUCAGGUAGAUUGCUU
UCUUUGGCAUGUCCGCAAAC-3′
(89 mer) (Hairpin)
5′ppp-dsRNA (19 mer, double stranded)
(SEQ ID NO: 112)
5′-pppGCAUGCGACCUCUGUUUGA-3′
(SEQ ID NO: 113)
3′-CGUACGCUGGAGACAAACU-5′

### Poly (dA:dT) (Double Stranded RNA)

### Poly(I:C)—HMW (High Molecular Weight) (Double Stranded RNA, has an Average Size of 1.5-8 kb)

### Poly(I:C)-LMW (Low Molecular Weight) (Double Stranded RNA, Average Size is 0.2-1 kb)

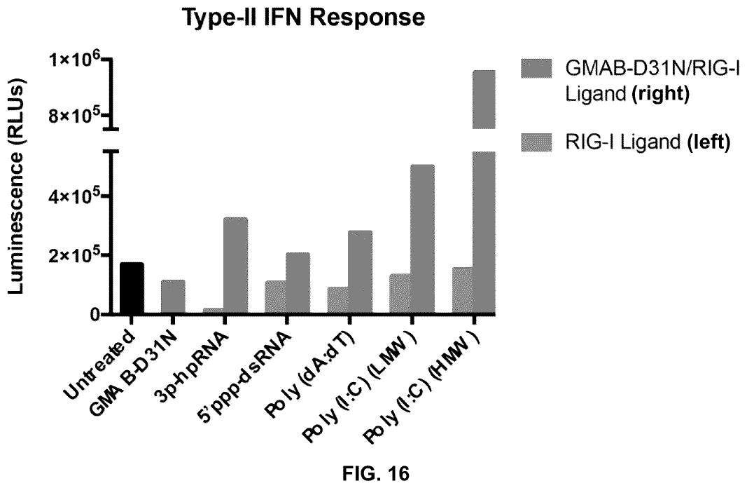

In all cases, RIG-I ligands alone did not stimulate IFN-γ secretion. Delivery of RIG-ligands with 3E10-D3IN, however, stimulated IFN-γ secretion above controls, with the highest secretion observed for poly (I:C), both low and high molecular weight (LMW and HMW), as shown in FIG. 16.

### Example 10-3E10 (D31N) Binds Poly-Deoxyribonucleotides with Greater Affinity than 3E10

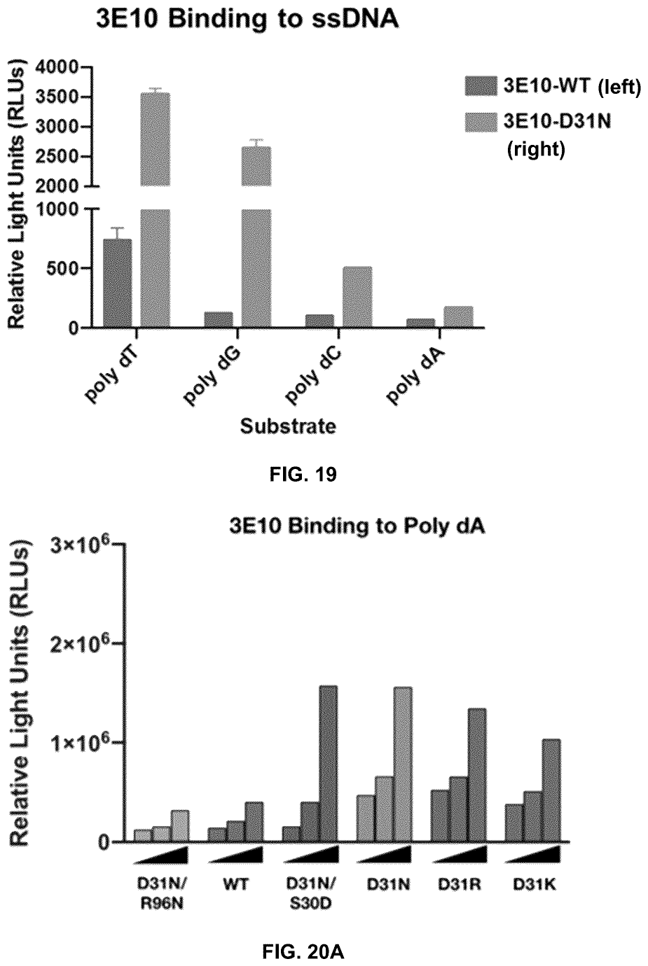

The binding affinities of 3E10 and 3E10 (D3IN) for various single-stranded poly-deoxyribonucleotides was investigated as described in Example 7. As shown in FIG. 19, 3E10 (D3IN) binds to each species of poly-deoxyribonucleotides with significantly stronger affinity than does 3E10. 3E10 antibodies showed a clear preference for binding poly-dT over the other three mono-polynucleotides. 3E10 (D31N) antibodies showed significantly greater binding affinity for poly-dT and poly-dG than poly-dC and poly-dA.

### Example 11-3E10 Variants Bind Poly-Deoxyribonuleotides with Different Nucleotide Preferences and Affinities

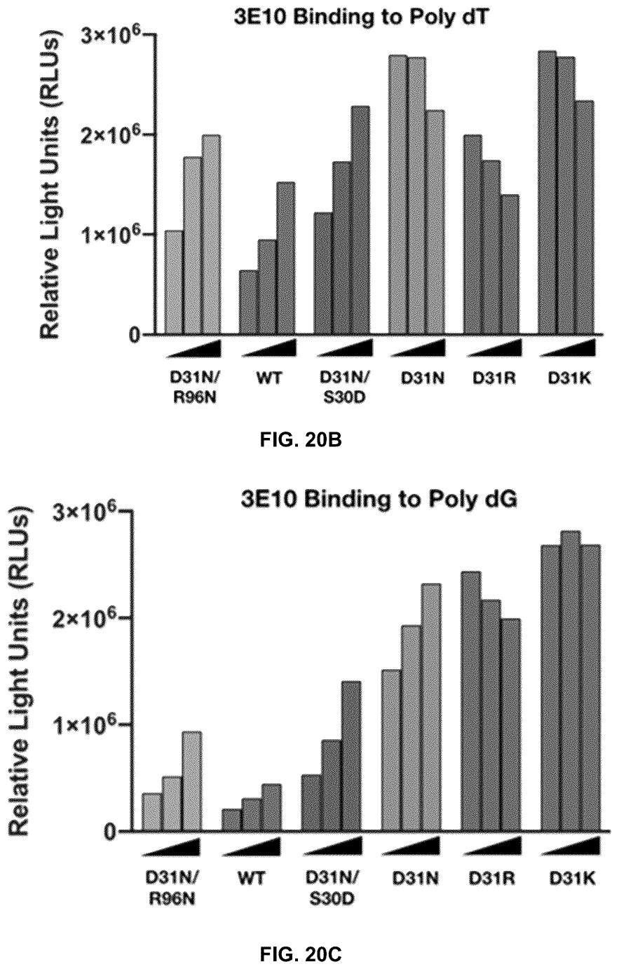

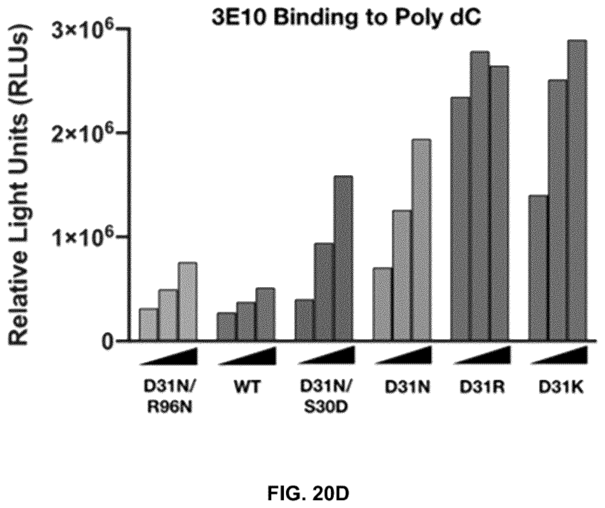

The binding affinities of 3E10 and 3E10 variants: (i) D31N/R96N, (ii) D31N/S30D, (iii) D31N, (iv) D31R, and (v) D31K for various single-stranded poly-deoxyribonucleotides was investigated as described in Example 7. As shown in FIGS. 20A-20D, in all cases other than poly-dA, the 3E10-D31K variant demonstrated superior binding relative to the parental 3E10 and all 3E10 variants. Doses for each variant from left to right were 100 nM, 200 nM, and 400 nM of 3E10 or 3E10 variant antibody.

### Example 12-3E10 and 3E10 (D31N) Protects mRNA Against RNA Degradation

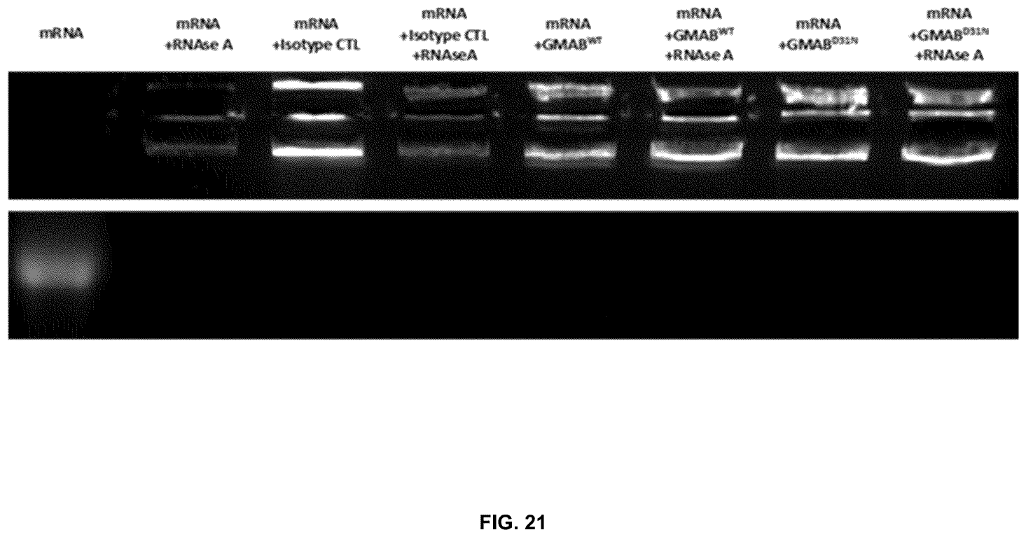

3E10 and 3E10 (D31N)-based mRNA protection was further investigated. Briefly, complexes of 3E10 or 3E10 (D3IN) and GFP_mRNA (SEQ ID NO:110) were formed by mixing 3E10 or 3E10 (D31N) and mRNA at a 20:1 molar ratio. The free mRNA, free mRNA complexed with a generic mouse monoclonal antibody (isotype CTL), and the antibody complexes were then incubated RNAse A as described in Example 6. Gel electrophoresis analysis of the reactions was performed (FIG. 21). As shown in FIG. 21, free mRNA, with or without “isotype CTL,” was degraded by incubation with RNAse A. However, 3E10 and 3E10 (D3IN) protected the mRNA from RNAse A-mediated degradation.

### Example 13-3E10-D31N is Internalized and Associates with gDNA In Vivo

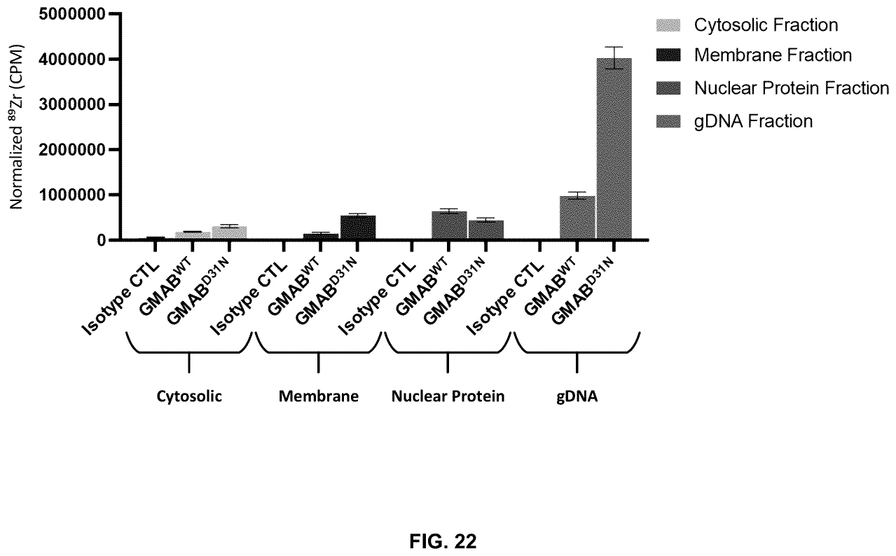

Internalization and cellular location experiments for 3E10-D3IN were investigated. Isotype control, 3E10-WT (GMABWT), and 3E10-D3IN (GMABD3IN) antibodies were labeled with ⁸⁹Zr and administered to cells in vivo. After an amount of time, cellular components (cytosol, membrane, nuclear protein, and gDNA) were fractionated and assayed for ⁸⁹Zr signal (Counts per Minute, CPM). As shown in FIG. 22, the majority of the internalized GMABWT and GMABD3IN antibodies localized to the nucleus and were found associated with both the nuclear protein fraction of the nucleus and the gDNA fraction. However, a larger portion of GMABD3IN associated with the gDNA fraction than did GMABWT, suggesting that GMABD3IN localizes more readily to chromatin than does GMABWT

### Example 14: 3E10 (D31N) Protects Dystrophin mRNA Against RNA Degradation

It was investigated whether 3E10-D3IN would protect mRNA encoding dystrophin from enzymatic degradation when complexed, and whether larger stochiometric amounts of 3E10-D3IN were necessary. Briefly, complexes of 3E10-D3IN and a 14 kb mRNA encoding full-length human dystrophin, were formed by mixing 3E10-D3IN and mRNA at 1:1, 2:1, 5:1, 10:1, 20:1, and 100: 1 molar ratios (3E10:mRNA). The free mRNA and the 3E10-mRNA complexes were then incubated with 6 μg/mL RNAse A for 10 minutes at 37° C. with the addition proteinase K to facilitate protein degradation. FIG. 23 shows agarose gel electrophoresis analysis of the protection assays. As shown in FIG. 15, free dystrophin mRNA, as well as dystrophin mRNA complexed at 1:1, 2:1, 5:1, and 10:1 molar ratios (3E10:mRNA) was completely degraded by incubation with RNAse A. However, as shown in FIG. 15, complexing the dystrophin mRNA with 3E10 at a molar ratio of 20:1 and 100:1 afforded increasing protection of the mRNA from degradation by RNAse A, as indicated by bands migrating at a similar distance as undegraded mRNA on the gel. These results, coupled with those of Example 6, suggest that 3E10 protects polynucleotides in a size-dependent manner.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the attached claims, along with the full scope of equivalents to which such claims are entitled.

What is claimed is:

1. A pharmaceutical composition comprising a therapeutically effective amount of a complex formed between (i) a 3E10 antibody or variant thereof, or antigen-binding fragment thereof; and (ii) a therapeutic polynucleotide comprising a 3E10 or 3E10 variant binding domain that is preferably bound by the 3E10 or 3E10 variant antibody.

2. The pharmaceutical composition of claim 1, wherein the 3E10 antibody or variant thereof, or antigen-binding fragment thereof comprises:
(a) a light chain variable region (VL) complementarity determining region (CDR) 1 comprising the amino acid sequence of 3E10-VL-CDR1 (SEQ ID NO:9),
(b) a VL CDR2 comprising the amino acid sequence of 3E10-VL-CDR2 (SEQ ID NO:10),
(c) a VL CDR3 comprising the amino acid sequence of 3E10-VL-CDR3 (SEQ ID NO:11),
(d) a heavy chain variable region (VH) CDR1 comprising the amino acid sequence of 3E10-VH-CDR1a (SEQ ID NO:16),
(e) a VH CDR2 comprising the amino acid sequence of 3E10-VH-CDR2 (SEQ ID NO:4), and
(f) a VH CDR3 comprising the amino acid sequence of 3E10-VH-CDR3 (SEQ ID NO:5).

3-14. (canceled)

15. The pharmaceutical composition of claim 1, wherein:
the 3E10 or 3E10 variant binding domain of the therapeutic polynucleotide has a first length and a first nucleotide sequence; and
the affinity of the 3E10 antibody or variant thereof, or antigen-binding fragment thereof for the 3E10 or 3E10 variant binding domain is at least 25% greater than the average affinity of the 3E10 antibody or variant thereof, or antigen-binding fragment thereof for a second polynucleotide having the first length and a second, random nucleotide sequence.

16. The pharmaceutical composition of claim 10, wherein the 3E10 or 3E10 variant binding domain comprises a first mononucleotide repeat sequence of at least 10 nucleotides in length.

17. The pharmaceutical composition of claim 16, wherein:
the VH CDR 1 has the amino acid sequence of 3E10-VH-CDR1_D3IN (SEQ ID NO:15);
the therapeutic polynucleotide is single-stranded DNA; and
the first mononucleotide repeat sequence is a poly-dT sequence.

18. The pharmaceutical composition of claim 16, wherein:
the VH CDR 1 has the amino acid sequence of 3E10-VH-CDR1_D31N (SEQ ID NO:15);
the therapeutic polynucleotide is single-stranded RNA; and
the first mononucleotide repeat sequence is a poly-rl sequence.

19. The pharmaceutical composition of claim 16, wherein:
the VH CDR 1 has the amino acid sequence of 3E10-VH-CDR1_D3IN (SEQ ID NO:15);
the therapeutic polynucleotide is single-stranded RNA; and
the first mononucleotide repeat sequence is a poly-rG sequence.

20. The pharmaceutical composition of claim 6, wherein:
the VH CDR 1 has the amino acid sequence of 3E10-VH-CDR1_D3IN (SEQ ID NO:15);
the therapeutic polynucleotide is single-stranded DNA; and
the 3E10 or 3E10 variant binding domain has a dT content of greater than 25%.

21. The pharmaceutical composition of claim 6, wherein:
the VH CDR 1 has the amino acid sequence of 3E10-VH-CDR1_D3IN (SEQ ID NO:15);
the therapeutic polynucleotide is single-stranded DNA; and
the 3E10 or 3E10 variant binding domain has a dA content of less than 25%.

22. The pharmaceutical composition of claim 6, wherein:
the VH CDR1 has the amino acid sequence of 3E10-VH-CDR1_D3IN (SEQ ID NO:15);
the therapeutic polynucleotide is single-stranded RNA; and
the 3E10 or 3E10 variant binding domain has a rI content of greater than 25%.

23. The pharmaceutical composition of claim 6, wherein:
the VH CDR 1 has the amino acid sequence of 3E10-VH-CDR1_D3IN (SEQ ID NO:15);
the therapeutic polynucleotide is single-stranded RNA; and
the 3E10 or 3E10 variant binding domain has a rG content of greater than 25%.

24. The pharmaceutical composition of claim 6, wherein:
the VH CDR 1 has the amino acid sequence of 3E10-VH-CDR1_D3IN (SEQ ID NO:15);
the therapeutic polynucleotide is single-stranded RNA; and
the 3E10 or 3E10 variant binding domain has a combined rl and rG content of greater than 25%.

25. The pharmaceutical composition of claim 22, wherein:
the VH CDR 1 has the amino acid sequence of 3E10-VH-CDR1_D3IN (SEQ ID NO:15);
the therapeutic polynucleotide is single-stranded RNA; and
the 3E10 or 3E10 variant binding domain has a rA content of less than 25%.

26.-28. (canceled)

29. The pharmaceutical composition of claim 1, wherein the therapeutic polynucleotide comprises an mRNA molecule.

30.-32. (canceled)

33. The pharmaceutical composition of claim 1, wherein the therapeutic polynucleotide comprises a DNA molecule.

34. The pharmaceutical composition of claim 1, wherein the therapeutic polynucleotide comprises an siRNA targeting an mRNA transcript.

35. (canceled)

36. The pharmaceutical composition of claim 1, wherein the therapeutic polynucleotide comprises an antisense oligonucleotide targeting an mRNA transcript.

37-105. (canceled)

106. A pharmaceutical composition comprising a therapeutically effective amount of a composition comprising a complex formed between (i) a 3E10 antibody or variant thereof, or antigen-binding fragment thereof, and (ii), a therapeutic polynucleotide wherein:
the therapeutic polynucleotide comprises a first codon-altered nucleotide sequence encoding a therapeutic polypeptide; and
the 3E10 antibody or variant thereof, or antigen-binding fragment thereof has a greater affinity for the first codon-altered nucleotide sequence than for a second nucleotide sequence that encodes the therapeutic polypeptide using a same coding sequence for the therapeutic polypeptide as found in a genome for the species of the subject.

107-180. (canceled)

181. A method for delivering a therapeutic polynucleotide to a tissue of a subject in vivo, the method comprising parenterally administering a pharmaceutical composition comprising a therapeutically effective amount of a complex formed between (i) a 3E10 antibody or variant thereof, or antigen-binding fragment thereof; and (ii) a therapeutic polynucleotide comprising a 3E10 or 3E10 variant binding domain that is preferably bound by the 3E10 or 3E10 variant antibody to the subject.

182-198. (canceled)

199. The pharmaceutical composition of claim 1, wherein the 3E10 antibody or variant thereof, or antigen-binding fragment thereof comprises:
(a) a light chain variable region (VL) complementarity determining region (CDR) 1 comprising the amino acid sequence of 3E10-VL-CDRm (SEQ ID NO:60),
(b) a VL CDR2 comprising the amino acid sequence of 3E10-VL-CDR2m (SEQ ID NO:62),
(c) a VL CDR3 comprising the amino acid sequence of 3E10-VL-CDR3m (SEQ ID NO:63),
(d) a heavy chain variable region (VH) CDR1 comprising the amino acid sequence of 3E10-VH-CDR1m (SEQ ID NO:58),
(e) a VH CDR2 comprising the amino acid sequence of 3E10-VH-CDR2m (SEQ ID NO:59), and
(f) a VH CDR3 comprising the amino acid sequence of 3E10-VH-CDR3m (SEQ ID NO:60).
